(19) United States
(12) Patent Application Publication
(10) Pub. No.: US 2024/0209354 A1
(43) Pub. Date: Jun. 27, 2024

(54) MULTIPLEX CRISPR/Cas9-MEDIATED TARGET GENE ACTIVATION SYSTEM

(71) Applicant: Salk Institute for Biological Studies, La Jolla, CA (US)

(72) Inventors: Juan Carlos Izpisua Belmonte, La Jolla, CA (US); Chao Wang, San Diego, CA (US); Pradeep Reddy, San Diego, CA (US); Hsin-Kai Liao, San Diego, CA (US)

(73) Assignee: Salk Institute for Biological Studies, La Jolla, CA (US)

(21) Appl. No.: 18/288,772

(22) PCT Filed: Apr. 28, 2022
(86) PCT No.: PCT/US2022/026805

§ 371 Date: Oct. 27, 2023

Related U.S. Application Data
(60) Provisional application No. 63/181,059, filed on Apr. 28, 2021.

(51) Int. Cl. C12N 15/11; C12N 9/22; C12N 15/86

(52) U.S. Cl. CPC C12N 15/111; C12N 9/22; C12N 15/86; C12N 2310/20; C12N 2750/14143

(57) ABSTRACT

Provided herein are multiplex crRNAs and multiplex sgRNAs, as well as RNA molecules thereof. Also provided are compositions and kits including the multiplex crRNAs and sgRNAs, which can be used in a multiplex targeted gene activation (mTGA) system. Also provided are methods that include administering a therapeutically effective amount of the mTGA system to a subject. In some examples, the method treats a disease associated with reduced or no expression of a gene, such as type I diabetes, Duchenne muscular dystrophy, a liver disease, or acute kidney disease.

### CROSS REFERENCE TO RELATED APPLICATIONS

This claims the benefit of U.S. Provisional Application No. 63/181,059, filed Apr. 28, 2021, which is incorporated by reference herein.

### FIELD

This application provides multiplex CRISPR RNAs (crRNAs) and multiplex single guide RNAs (sgRNAs), as well as compositions and kits including multiplex crRNAs and multiplex sgRNAs, which can be used in a multiplex targeted gene activation (mTGA) system, for example, to increase expression of a gene, to reprogram a cell, or to treat a disease in vivo.

### BACKGROUND

Duchenne muscular dystrophy (DMD) is a lethal muscle wasting disease and one of the most frequent genetic disorders worldwide, affecting 1 in every 3,500 to 5,000 live male births. DMD leads to progressive muscle weakness, which ultimately results in respiratory and heart failure in the teen years (Blake et al. (2002) Physiological Reviews 82:291-329). DMD is caused by frameshift mutations in the dystrophin gene, and at least 726 different mutations have been identified across the entire coding region (Bladen et al. (2015) Hum Mutat 36:395-402). There are several mutational ‘hotspots’ within this gene, including exons 45-53, among which exon 51 is mutated most frequently, representing ˜13% of DMD cases. Currently, there is no effective therapy for DMD and transplanting muscle stem cells into damaged organs to stop disease progression has proven difficult. Due to the large size of the dystrophin gene (the cDNA is ˜14 kb), it has also proven challenging to deliver a functional dystrophin transgene to affected tissues via traditional virus-mediated gene therapies (Janghra et al. (2016) PloS one 11, e0150818; Sicinski et al. (1989) Science 244:1578-1580).

Recently, several groups have restored dystrophin gene function by using CRISPR/Cas9 technology to remove the mutated exons, thereby creating a shortened but functional version of the dystrophin gene (Amoasii et al. (2018) Science 362:86-91; Amoasii et al. (2017) Sci Transl Med 29:9(418); Bengtsson et al. (2017) Nat Commun 14:8, 14454; Long et al. (2016) Science 351:400-403; Moretti et al. (2020) Nat Med 26:207-214; Nelson et al. (2016) Science 351:403-407; Nelson et al. (2019) Nat Med 25:427-432; Tabebordbar et al. (2016) Science 351:407-411; Zhang et al. (2017) Sci Adv 3, e1602814). Although this method has shown promise, some exons within the dystrophin gene are important for protein function and cannot be removed to cure the disease. Only 55% of patients with DMD could potentially benefit from these exon skipping/excision therapies (Bladen et al. (2015) Hum Mutat 36:395-402). Thus, alternative approaches for restoring muscle function in DMD are needed, particularly approaches that are effective regardless of which dystrophin mutation is carried by the patient.

Utrophin is a functional analog of dystrophin and therefore can likely compensate for the loss of dystrophin in DMD patients (Rafael et al. (1998) Nat Gen 19, 79-82; Tinsley et al. (1996) Nature 384:349-353). Thus, a potential treatment strategy is upregulating utrophin in patients with DMD. The CRISPR/Cas9 system can be modified such that instead of inducing double-strand breaks in target DNA, the system induces targeted gene expression by recruiting transcriptional activation domains to a targeted promoter region (Qi et al. (2013) Cell 152:1173-1183; Liao et al. (2017) Cell 171:1495-1507 e1415). However, a major obstacle in implementing this system for the treatment of DMD is that utrophin induction by the CRISPR/Cas9 gene activation system has been limited and a more robust system is needed.

### SUMMARY

Provided herein are nucleic acid molecules (such as DNA molecules) encoding multiplex CRISPR RNAs (crRNAs) and multiplex single guide RNAs (sgRNAs). The encoded multiplex crRNAs include a first promoter operably linked to a nucleic acid molecule encoding a modified trans-activating CRISPR RNA (tracrRNA), a first cleavage site, a first nucleic acid molecule encoding a first crRNA, a second cleavage site, and a second nucleic acid molecule encoding a second crRNA. The modified tracrRNA encodes at least two modified MS2-binding loops. In some embodiments, the encoded multiplex crRNA further includes a second promoter operably linked to a third nucleic acid molecule encoding a crRNA or a dead guide RNA (dgRNA). In some examples, the second promoter and third crRNA (or dgRNA) are in reverse orientation relative to the first promoter. In some examples, the second promoter and third crRNA (or dgRNA) are located 5′ of the first promoter. In some examples, the first cleavage site is a pre-transfer RNA (pre-tRNA) and the second cleavage site is a self-cleaving ribozyme, such as a hammerhead ribozyme. In further examples, a crRNA, sgRNA or dgRNA disclosed herein include a targeting sequence complementary to a sequence within a promoter region of EEF1a2 (Eukaryotic Translation Elongation Factor 1 Alpha 2), Fst (Follistatin), Pdx1(pancreatic and duodenal homeobox 1), klotho, utrophin, interleukin 10, or Six2 (SIX Homeobox 2).

Also provided herein are nucleic acids (such as DNA molecules) encoding multiplex single guide RNAs (sgRNAs). The multiplex sgRNAs include a first nucleic acid molecule encoding, in reverse orientation, a first modified sgRNA operably linked to a first promoter and a second nucleic acid molecule encoding in forward orientation a second modified sgRNA operably linked to a second promoter. The first and the second modified sgRNAs encode at least two modified MS2-binding loops. In some embodiments, the multiplex sgRNA further include a third nucleic acid molecule located 3′ of the second nucleic acid molecule, wherein the third nucleic acid encodes in forward orientation a first cleavage site and a third modified sgRNA. In some embodiments, the multiplex sgRNA further includes a fourth nucleic acid molecule located 5′ of the first nucleic acid molecule, wherein the fourth nucleic acid molecule encodes in reverse orientation a second cleavage site and a fourth modified sgRNA. The third and the fourth modified sgRNAs encode at least two modified MS2-binding loops. In some examples, the first and/or second cleavage site encode a pre-tRNA. In some examples, the sgRNAs disclosed herein include a targeting sequence complementary to a sequence within a promoter region of EEF1a2, Fst, Pdx1, klotho, utrophin, interleukin 10, or Six2. In some examples, the sgRNAs are dgRNAs.

Also provided are RNA molecules encoded by the disclosed nucleic acids, and vectors that include the disclosed nucleic acids (such as the nucleic acids encoding the multiplex crRNAs or multiplex sgRNAs), such as a viral vector, for example, an AAV vector such as an AAV9 vector. Also provided are compositions including the disclosed nucleic acids, or RNA molecules thereof, or the disclosed vectors, and a pharmaceutically acceptable carrier.

Also provided are kits that include the disclosed nucleic acid, RNA, composition, or viral vector, and a nucleic acid encoding a Cas9 protein or dead Cas9 (dCas9) protein, and/or a nucleic acid encoding a MS2-transcriptional activator fusion protein.

Also provided is a multiplex targeted gene activation (mTGA) system. The system can include a first vector (such as a viral vector, e.g., AAV9) that includes a nucleic acid encoding a Cas9 or dCas9 and a second vector (such as a viral vector, e.g., AAV9) that includes a nucleic acid disclosed herein (such as a nucleic acid encoding a multiplex crRNA or multiplex sgRNA) and a nucleic acid encoding an MS2-transcriptional activator fusion protein (such as MS2-p65-HSF1).

Methods of using the disclosed nucleic acids, RNAs, compositions, viral vectors, kits, and mTGA system are also provided. The methods include administering a therapeutically effective amount of the disclosed mTGA system to a subject. In some examples, the method increases expression of at least one target gene in the subject, thereby increasing expression of at least one gene product. In some examples, the method treats a disease in the subject caused by, or associated with, reduced or no expression of a gene. In some examples, the target gene is a gene whose reduced expression causes the disease (a causative gene). In further examples, the target gene is a functional analog of a causative gene, and expression of the functional analog compensates for the loss of function of the causative gene. In some examples, the disease is muscular dystrophy and the causative gene is dystrophin and the target gene is utrophin. In some examples, the disease is a liver fibrosis or cirrhosis and the target gene is Foxa3, Gata4, HNF1a, and/or HNF4a.

The foregoing and other objects and features of the disclosure will become more apparent from the following detailed description, which proceeds with reference to the accompanying figures.

### BRIEF DESCRIPTION OF THE DRAWINGS

FIG. 1 shows an example coding multiplex CRISPR RNA (crRNA) construct 100 containing two crRNAs 101, 102.

FIGS. 2A-2B show exemplary coding multiplex crRNA constructs 100 containing two crRNAs and a third nucleic acid molecule 103 encoding a third crRNA or a dgRNA operably linked to a second promoter 111. The third nucleic acid molecule 103 can be located 3′ of the second crRNA (FIG. 2A) or 5′ of the first promoter (FIG. 2B). In some embodiments, the third nucleic acid molecule is located 5′ of the first promoter and is in reverse orientation relative to the first promoter (FIG. 2B).

FIGS. 3A-3E show example coding multiplex single guide RNA (sgRNA) constructs 200. FIGS. 3A, 3C and 3D show example DNA constructs containing two sgRNAs. FIGS. 3B and 3E show example DNA constructs containing three sgRNAs.

FIG. 4 shows an example coding multiplex single guide RNA (sgRNA) construct 200 containing four sgRNAs.

FIG. 5 shows utrophin activation of dgRNAs targeting different regions of the utrophin locus (the sequence shown is SEQ ID NO: 56).

FIG. 6A shows activation of utrophin (Utrn) as analyzed by qRT-PCR two days after transfection. Cas9-expressing N2a (N2aCas9) cells were transfected with the indicated combinations of utrophin targeting dgRNAs and a plasmid containing MPH. FIG. 6B shows dgRNA activation of Eef1a2 expression.

FIG. 7 shows a western blot (top) and relative protein levels (bottom) of Utrn in N2a^{Cas9} cells. dgEef1a2 and dgUtrnNT2 in combination significantly enhances the upregulation of utrophin.

FIG. 8 shows a schematic of AAV vectors containing one sgRNA (top), or multiplex sgRNAs (middle and bottom).

FIG. 9 shows the efficiency of different promoters in mouse N2 cells. Cas9-expressing N2a (N2a^{Cas9}) cells were transfected with the indicated plasmid and a plasmid containing MPH. Activation of Fst was analyzed by qRT-PCR 2 days after transfection.

FIG. 10 shows activation efficiency of UtnNT2, Eef1a2, and MyoD using hU6, mU6, H1, or 7SK promoters.

FIG. 11 shows the induction of targeted gene expression using a two multiplex sgRNA system when the second sgRNA (dgFst) is in forward (circles) or reverse (square) orientation relative to the first sgRNA (dgUtrn).

FIG. 12 shows a schematic of recombination that occurs when both sgRNAs are in forward orientation (top) and a gel electrophoresis image (bottom). Presence of the “low band” in the gel confirms presence of unwanted recombination product when both sgRNAs are in the forward orientation. Recombination was verified by Sanger sequencing (see FIG. 13). Blue arrows indicate primer locations for PCR amplification.

FIG. 13 shows Sanger sequencing confirming the presence of recombination product. The top sequence is SEQ ID NO: 57, the bottom sequence is SEQ ID NO: 58.

FIG. 14 shows a schematic of duo-dgRNAs using direct repeat (DR) or inverse repeat (IR) orientation. Fold activation of target genes by duo-dgRNAs in DR (circle) or IR (square) orientation is shown below.

FIG. 15 shows that a truncated product is produced when duo-dgRNAs are in direct repeat orientation, indicating unwanted recombination.

FIG. 16 shows a schematic of skeletal muscle-specific mTGA constructs with duo-dgRNAs oriented as inverted repeats. Below is an exemplary design for an in vivo experiment.

FIG. 17 shows myofiber damage in TA muscles as indicated by EBD uptake. Damaged myofibers accumulate EBD, and thus show stronger fluorescence. TA muscle mass is also shown (top right).

FIGS. 18A and 18 B show expression of targeted genes. FIG. 18A shows that AAV9-dgUtrnT2-dgFst-MPH treatment increased the expression of utrophin and Fst by 1.8-fold and 10-fold, respectively. FIG. 18B shows that AAV9-dgUtmNT2-dgEef1a2-MPH treatment increased the expression of utrophin and Eef1a2 by 2.6-fold and 2.2-fold, respectively.

FIG. 19 shows a western blot (left) and relative protein levels (right) following in vivo treatment. The results show that AAV9-dgUtmNT2-dgEef1a2-MPH (U-E) treatment upregulated expression of utrophin by 3.7-fold, while AAV9-dgUtrnT2-dgFst-MPH (U-T) treatment upregulated utrophin by 1.5 fold.

FIG. 20 shows immunostaining of utrophin.

FIG. 21 shows a schematic of three multiplex sgRNAs driven by three individual RNA polymerase III promoters. Gel electrophoresis shows that unwanted recombination occurred in a construct with three promoters (lower band). Blue arrows indicate primer location for amplification. Recombination was verified by Sanger sequencing (see FIG. 22).

FIG. 22 shows Sanger sequencing confirming unwanted recombination product in a construct with three promoters. The sequence shown is SEQ ID NO: 59.

FIG. 23 shows a comparison of fold-activation using a system with two individual promoters driving expression of two gRNAs (bottom schematic), or a system with one promoter driving expression of two gRNAs separated by a tRNA (top schematic).

FIG. 24 compares gene activation by the indicated constructs using N2a^{Cas9} cells.

FIG. 25 shows a comparison of recombination of two sgRNA systems with either two promoters (top schematic) or 1 promoter and a tRNA cleavage site (bottom schematic). Gel electrophoresis and Realtime qPCR results indicate less recombination occurred in the construct containing 1 promoter with the tRNA. Blue arrows indicate primer location for amplification.

FIG. 26 shows activation efficiency of the hU6-tRNA and hU6-H1 constructs.

FIG. 27 shows a gel electrophoresis image indicating that recombination events occur less in the hU6-tRNA construct than in the hU6-H1 construct.

FIG. 28 shows qPCR results of the ratio of tRNA or H1 versus hU6 in plasmids and in AAV collected from the C2C12^{Cas9} cells.

FIG. 29 shows efficient activation of MyoD, Mef2b and Pax7 in 3T3L1^{Cas9} cells treated with the indicated mTGA construct (containing dgMyoD, dgMef2b, and dgPax7).

FIG. 30 shows a comparison of the UtrnT2 TGA system (one sgRNA) and UtrnTriple multiplex TGA (mTGA) system (three sgRNAs). N2a^{Cas9} cells were transfected with AAV vectors containing the single TGA (UtrnT2) and mTGA (UtrnTriple) system. Activation of utrophin was analyzed by qRT-PCR 2 days after transfections. C2C12^{Cas9} cells were transduced with AAV containing the single and mTGA systems. Activation of utrophin was analyzed by qRT-PCR 10 days after transduction.

FIG. 31 shows that the multiplex TGA system activates the expression of multiple genes simultaneously in tibialis anterior (TA) muscles of Cas9+Mdx mice.

FIG. 32 shows gene activation using an mTGA construct containing four gRNAs.

FIGS. 33A-33B shows that the mTGA system enhances expression of utrophin in vivo.

FIG. 33A: Cas9-expressing WT mice were injected with AAVs containing the single-gRNA TGA (UtrnT2) or mTGA (UtrnTriple) system. Activation of utrophin was analyzed by qRT-PCR two months after injection (n=5). FIG. 33B: shows a western blot analysis of utrophin in tibialis anterior (TA) muscles injected with AAV containing single TGA (gUtrnT2-MPH), mTGA (gUtrnTriple-MPH), or MPH only. Hsp90 is the loading control.

FIGS. 34A-34B shows RNA-seq analysis of tibialis anterior (TA) muscles injected with AAV containing gUtrnTriple-MPH, or MPH only (FIG. 34A). FIG. 34B shows immunostaining for utrophin in TA muscles injected with indicated AAV. Scale bar=50 μm.

FIG. 35 shows the experimental design for the grip strength assay (top) and grip strength of the indicated mice with the indicated AAV treatment (bottom). 60 continuous grip strength tests were performed for each mouse. Reads were averaged for every 10 tests.

FIG. 36 shows an evaluation of sarcolemmal integrity by intraperitoneal injections of EBD in mice with the indicated treatment. EBD accumulates in damaged cells. Two hours after EBD injection, mice were subjected to treadmill running for 2 min with a speed of 6 m/min, followed by 2 min of rest. Treadmill running was repeated 3 times. High level of EBD uptake indicates muscle damage. Treatment with the mTGA system (UtrnTriple) strikingly ameliorated myofiber break during contraction.

FIG. 37 shows that the mTGA system enhances the expression of utrophin in Mdx mice. Cas9-expressing Mdx mice were injected with AA Vs containing the single sgRNA TGA system (UtrnT2) or mTGA system (UtrnTriple). Activation of utrophin was analyzed by qRT-PCR two months after injection (n=4).

FIG. 38 shows Cas9-expressing Mdx mice injected with AAVs containing the single sgRNA TGA system (UtrnT2) or mTGA system (UtrnTriple). Immunostaining for utrophin in TA muscles injected with indicated AAV.

FIG. 39 shows EBD uptake into TA muscles of mdx mice two months after mTGA treatment. Extensive EBD uptake was found in mdx mice with control treatment, while EBD uptake is significantly alleviated in mTGA-treated mice. In addition, Utrn immunostaining confirms activation of utrophin.

FIGS. 40A and 40B shows quantification of expression of utrophin by qPCR (FIG. 40A) and western blot (FIG. 40B) of TA muscles treated with control (MPH) and the mTGA system (UtrnTriple).

FIG. 41A shows the experimental design. TA muscles of Cas9/mdx mice are injected with 1×10¹¹ GC AAV9-MPH, AAV9-hU6-dgUtmT2-MPH, AAV9-UtrnDual, or AAV9-UtrnTriple. FIG. 41B shows mRNA level of utrophin two months after AAV injection.

FIGS. 42 and 43 show chromatin-immunoprecipitation (ChIP) qRT-PCR of TA muscle samples.

FIG. 44A shows the experimental design. TA muscles of the idCas9 mice were co-injected with AAV containing a luciferase reporter in which luciferase was placed downstream of a dgRNA (dgLuc) binding site and AAV containing a dgLuc-CAG-MPH sequence. Then, Dox water (1 mg/ml) was added and removed at an interval of 1-week or 2-weeks. FIG. 44B shows that the luciferase signal was induced 1-week after Dox administration, and turned back to basal levels 2-weeks after administration.

FIG. 45 shows endogenous activation of utrophin in idCas9 mice injected with 1×10¹¹ GC AAV9-UtrnTriple or AAV9-MPH. Mice were administered continuous Dox for 30 days (30 on), continuous Dox for 60 days (60 on), or continuous Dox for 30 days following 30 days of no Dox (30 off).

FIG. 46A shows experimental design for co-injection of AAV9-dCas9 and AAV9-UtrnTriple or AAV9-MPH. Muscle samples were collected 13-months after treatment. FIG. 46B shows a 3-fold increase of utrophin was found in samples treated with the mTGA system. FIG. 46C shows immunostaining of utrophin, verifying Utrn activation.

FIGS. 47A and 47B show H&E staining (FIG. 47A) and Mallory's trichrome staining (FIG. 47B) to evaluate the histopathological phenotypes of muscle samples.

FIG. 48 shows dgUtrnNT2-Eef1a2, dgUtrnNT2-dgUtrT2-dgUtrnT16 (UtrnTriple), and UtrnDual-Eefla2 mTGA constructs.

FIG. 49A shows expression of Eef1a2 and utrophin in TA muscles of mdx mice two months after treatment with dgUtrnNT2-Eef1a2, UtrnTriple, UtrnDual-Eefla2, or MPH. FIG. 49B shows Utrn protein levels.

FIG. 50A is a schematic showing intramuscular injection of MPH or the dual-AAV system to multiple muscles of 2-month-old mdx mice. FIG. 50B shows serum creatine kinase activity two month after AAV treatment.

FIGS. 51A and 51B show that mTGA treatment increases activity and endurance of mdx mice compared to control mice (MPH). FIG. 51A shows the results of an open field test. FIG. 51B shows the results of a treadmill test.

FIG. 52 shows a sequencing map showing that recombination in the single promoter-tRNA construct happens between the 1^{st} and 4^{th} MS2 loop. Unlabeled bars indicate MS2 loops. The top sequence is SEQ ID NO: 60, the bottom sequence is SEQ ID NO: 61.

FIGS. 53A and 53B show activation of target genes using crispr RNAs (crRNA) and a modified trans-activating crispr RNA containing the 2 MS2 loop (tracrRNA-M2). The crRNA-tRNA-tracrRNA-M2 construct was able to activate the target gene, while its activation efficiency was 2.8-fold lower than dgRNA (FIG. 53A). When 2 crRNA were driven by two different U6 promoters, only the crRNA that shared the same promoter with tracrRNA-M2 had strong activation efficiency (FIG. 53B).

FIG. 54 shows the design and testing of an alternative mTGA system utilizing tRNAs and/or hammerhead RNAs to between tracrRNA and crRNA elements. Gene1 is Fst and Gene2 is utrophin.

FIG. 55 shows gel electrophoresis indicating that no recombination occurs in the construct containing a tracrRNAM2 and two crRNA1 (crFst) and crRNA2 (crUtrn).

FIG. 56A shows the activation efficiency of the AAVDJ-hU6-tracrRNA-M2-tRNA-crFst-HDV-HH-crUtrn-MPH was not higher than AAVDJ-hU6-dgUtrnT2-tRNA-dgFst-MPH in C2C12^{Cas9} cells. FIG. 56B shows in vivo activation of utrophin two months after intramuscular injections of different concentrations of AAV9-MPH, AAV9-UtrnTriple, or AAV9-UtrnTriple-crRNA, into TA muscles of Cas9/mdx mice.

FIG. 57 shows luciferase expression to trace distribution of AAV after tail vein injection at the indicated titers.

### SEQUENCE LISTING

Any nucleic acid and amino acid sequences listed herein or in the accompanying Sequence Listing are shown using standard letter abbreviations for nucleotide bases and amino acids, as defined in 37 C.F.R. § 1.822. In at least some cases, only one strand of each nucleic acid sequence is shown, but the complementary strand is understood as included by any reference to the displayed strand. The Sequence Listing is submitted as an ASCII text file, “Sequence.txt,” created on Apr. 27, 2022, 81,920 bytes, which is incorporated by reference herein. In the accompanying sequence listing:

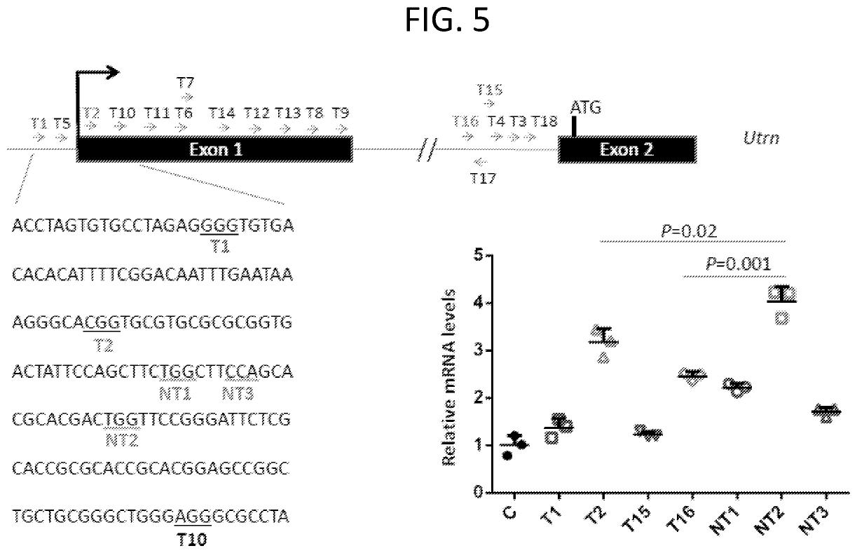

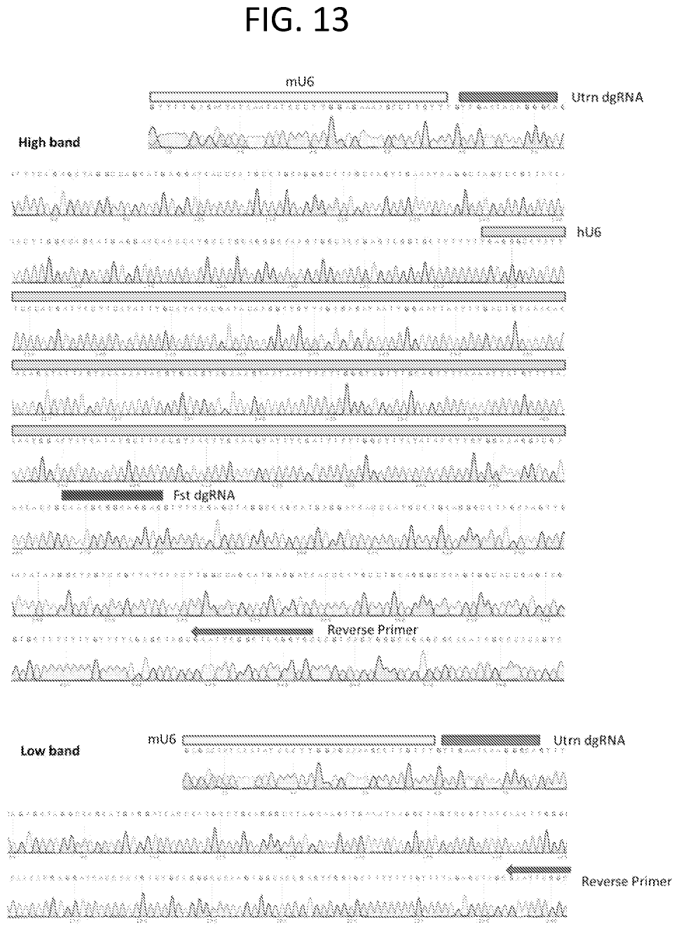

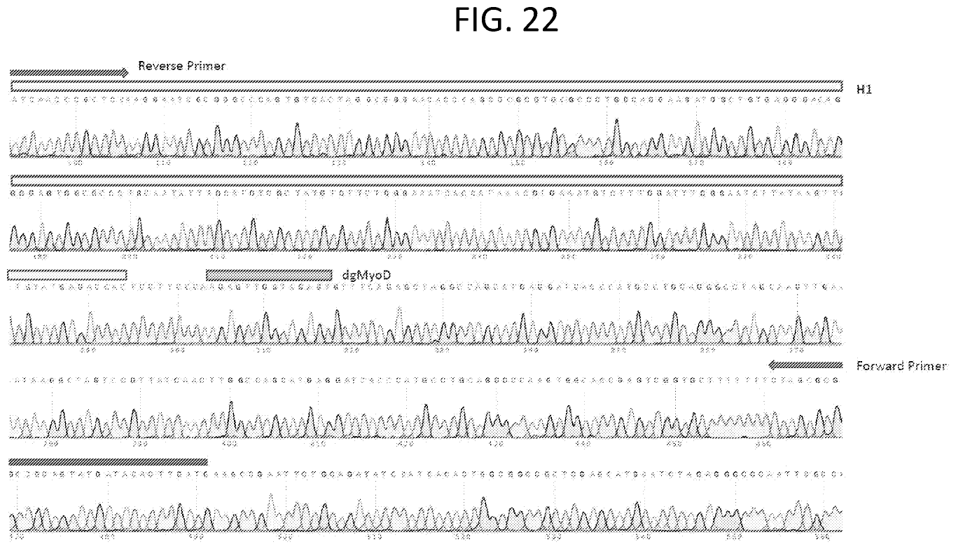

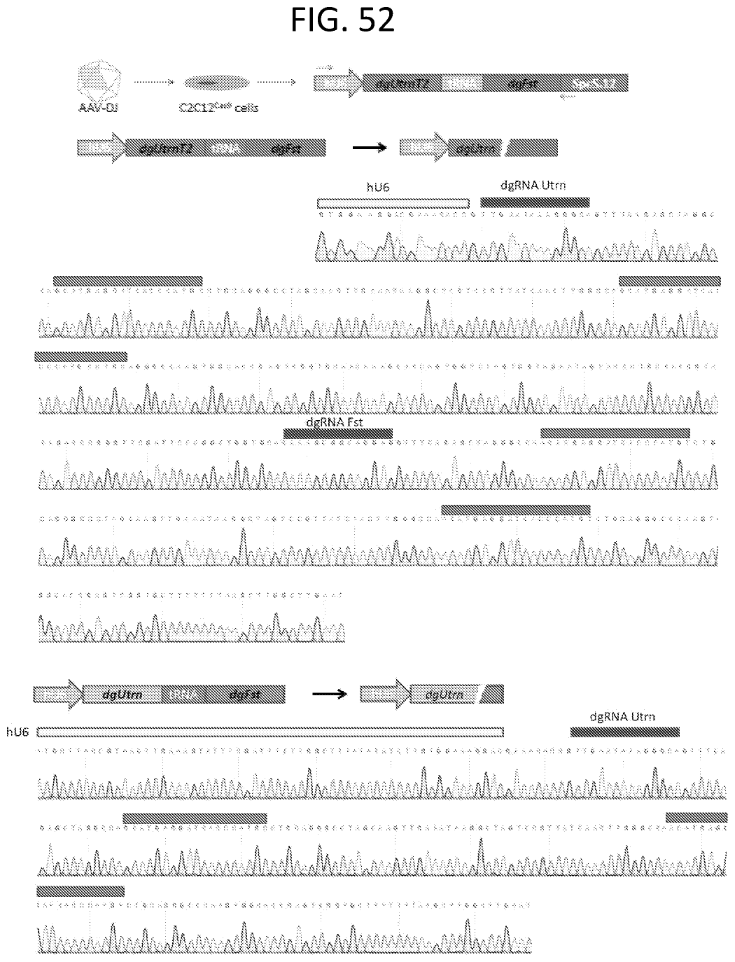

SEQ ID NO: 1 is an exemplary DNA sequence encoding tracrRNA-tRNA-UT2-HH-UT16
multiplex crRNAs.
GAACCATTCAAAACAGCATAGCAAGTTAAAATAAGGCTAGTCCGTTATCAACTTGAAA
AAGTGGCACCGAGTCGGTGCGGGAGCGGCCAGCATGAGGATCACCCATGCCTGCAGG
GCCGCCACGAGCGGGGCCAACATGAGGATCACCCATGTCTGCAGGGCCCCGCTCGTGT
TCCCAACAAAGCACCAGTGGTCTAGTGGTAGAATAGTACCCTGCCACGGTACAGACCC
GGGTTCGATTCCCGGCTGGTGCAGAGAGCAGCAGTTGGTTTTAGAGCTATGCTGTTTTG
GGCCGGCATGGTCCCAGCCTCCTCGCTGGCGCCGGCTGGGCAACATGCTTCGGCATGG
CGAATGGGACATTCAACTGATGAGTCCGTGAGGACGAAACGAGTAAGCTCGTCTTGAA
TAAAGGGCAGTTTTAGAGCTATGCTGTTTTGTTTTTTT
SEQ ID NO: 2 is an exemplary DNA sequence encoding dgUtnNT2-mU6-hU6-tracrRNA-tRNA-
crUT2-HH-crUT16 multiplex crRNAs with a dgRNA (“UtrTriple-crRNA”).
AAAAAAAGCACCAGCCGGGAATCGAACCCGGGTCTGTACCGTGGCAGGGTACTATTCT
ACCACTAGACCACTGGTGCTTTGTTGCACCGACTCGGTGCCACTTGGCCCTGCAGGCAT
GGGTGATCCTCATGCTGGCCAAGTTGATAACGGACTAGCCTTATTTCAACTTGCTAGGC
CCTGCAGGCATGGGTGATCCTCATGCTGGCCTAGCTCTGAAACGTCGTGCGTGCTGGC
AAACAAGGCTTTTCTCCAAGGGATATTTATAGTCTCAAAACACACAATTACTTTACAGT
TAGGGTGAGTTTCCTTTTGTGCTGTTTTTTAAAATAATAATTTAGTATTTGTATCTCTTA
TAGAAATCCAAGCCTATCATGTAAAATGTAGCTAGTATTAAAAAGAACAGATTATCTG
TCTTTTATCGCACATTAAGCCTCTATAGTTACTAGGAAATATTATATGCAAATTAACCG
GGGCAGGGGAGTAGCCGAGCTTCTCCCACAAGTCTGTGCGAGGGGGCCGGCGCGGGC
CTAGAGATGGCGGCGTCGGATCGAGGGCCTATTTCCCATGATTCCTTCATATTTGCATA
TACGATACAAGGCTGTTAGAGAGATAATTGGAATTAATTTGACTGTAAACACAAAGAT
ATTAGTACAAAATACGTGACGTAGAAAGTAATAATTTCTTGGGTAGTTTGCAGTTTTAA
AATTATGTTTTAAAATGGACTATCATATGCTTACCGTAACTTGAAAGTATTTCGATTTC
TTGGCTTTATATATCTTGTGGAAAGGACGAAACACCGGAACCATTCAAAACAGCATAG
CAAGTTAAAATAAGGCTAGTCCGTTATCAACTTGAAAAAGTGGCACCGAGTCGGTGCG
GGAGCGGCCAGCATGAGGATCACCCATGCCTGCAGGGCCGCCACGAGCGGGGCCAAC
ATGAGGATCACCCATGTCTGCAGGGCCCCGCTCGTGTTCCCAACAAAGCACCAGTGGT
CTAGTGGTAGAATAGTACCCTGCCACGGTACAGACCCGGGTTCGATTCCCGGCTGGTG
CAGAGAGCAGCAGTTGGTTTTAGAGCTATGCTGTTTTGGGCCGGCATGGTCCCAGCCT
CCTCGCTGGCGCCGGCTGGGCAACATGCTTCGGCATGGCGAATGGGACATTCAACTGA
TGAGTCCGTGAGGACGAAACGAGTAAGCTCGTCTTGAATAAAGGGCAGTTTTAGAGCT
ATGCTGTTTTGTTTTTTT
SEQ ID NO: 3 is an exemplary DNA sequence encoding a dgFst/dgUtrn multiplex sgRNAs.
AAAAAAAGCACCAGCCGGGAATCGAACCCGGGTCTGTACCGTGGCAGGGTACTATTCT
ACCACTAGACCACTGGTGCTTTGTTGCACCGACTCGGTGCCACTTGGCCCTGCAGGCAT
GGGTGATCCTCATGCTGGCCAAGTTGATAACGGACTAGCCTTATTTCAACTTGCTAGGC
CCTGCAGGCATGGGTGATCCTCATGCTGGCCTAGCTCTGAAACGTCGTGCGTGCTGGC
AAACAAGGCTTTTCTCCAAGGGATATTTATAGTCTCAAAACACACAATTACTTTACAGT
TAGGGTGAGTTTCCTTTTGTGCTGTTTTTTAAAATAATAATTTAGTATTTGTATCTCTTA
TAGAAATCCAAGCCTATCATGTAAAATGTAGCTAGTATTAAAAAGAACAGATTATCTG
TCTTTTATCGCACATTAAGCCTCTATAGTTACTAGGAAATATTATATGCAAATTAACCG
GGGCAGGGGAGTAGCCGAGCTTCTCCCACAAGTCTGTGCGAGGGGGCCGGCGCGGGC
CTAGAGATGGCGGCGTCGGATCGAGGGCCTATTTCCCATGATTCCTTCATATTTGCATA
TACGATACAAGGCTGTTAGAGAGATAATTGGAATTAATTTGACTGTAAACACAAAGAT
ATTAGTACAAAATACGTGACGTAGAAAGTAATAATTTCTTGGGTAGTTTGCAGTTTTAA
AATTATGTTTTAAAATGGACTATCATATGCTTACCGTAACTTGAAAGTATTTCGATTTC
TTGGCTTTATATATCTTGTGGAAAGGACGAAACACCGCAAAGCGGCAGGAGGTTTCAG
AGCTAGGCCAGCATGAGGATCACCCATGCCTGCAGGGCCTAGCAAGTTGAAATAAGG
CTAGTCCGTTATCAACTTGGCCAGCATGAGGATCACCCATGCCTGCAGGGCCAAGTGG
CACCGAGTCGGTGCTTTTTTT
SEQ ID NO: 4 is an exemplary DNA sequence encoding dgUtnNT2/dgUtrnT2/dgUtrnT16
multiplex sgRNAs (“UtrnTriple”).
AAAAAAAGCACCAGCCGGGAATCGAACCCGGGTCTGTACCGTGGCAGGGTACTATTCT
ACCACTAGACCACTGGTGCTTTGTTGCACCGACTCGGTGCCACTTGGCCCTGCAGGCAT
GGGTGATCCTCATGCTGGCCAAGTTGATAACGGACTAGCCTTATTTCAACTTGCTAGGC
CCTGCAGGCATGGGTGATCCTCATGCTGGCCTAGCTCTGAAACGTCGTGCGTGCTGGC
AAACAAGGCTTTTCTCCAAGGGATATTTATAGTCTCAAAACACACAATTACTTTACAGT
TAGGGTGAGTTTCCTTTTGTGCTGTTTTTTAAAATAATAATTTAGTATTTGTATCTCTTA
TAGAAATCCAAGCCTATCATGTAAAATGTAGCTAGTATTAAAAAGAACAGATTATCTG
TCTTTTATCGCACATTAAGCCTCTATAGTTACTAGGAAATATTATATGCAAATTAACCG
GGGCAGGGGAGTAGCCGAGCTTCTCCCACAAGTCTGTGCGAGGGGGCCGGCGCGGGC
CTAGAGATGGCGGCGTCGGATCGAGGGCCTATTTCCCATGATTCCTTCATATTTGCATA
TACGATACAAGGCTGTTAGAGAGATAATTGGAATTAATTTGACTGTAAACACAAAGAT
ATTAGTACAAAATACGTGACGTAGAAAGTAATAATTTCTTGGGTAGTTTGCAGTTTTAA
AATTATGTTTTAAAATGGACTATCATATGCTTACCGTAACTTGAAAGTATTTCGATTTC
TTGGCTTTATATATCTTGTGGAAAGGACGAAACACCGGACAATTTGAATAAAGGGCAG
TTTCAGAGCTAGGCCAGCATGAGGATCACCCATGCCTGCAGGGCCTAGCAAGTTGAAA
TAAGGCTAGTCCGTTATCAACTTGGCCAGCATGAGGATCACCCATGCCTGCAGGGCCA
AGTGGCACCGAGTCGGTGCAACAAAGCGCAAGTGGTTTAGTGGTAAAATCCAACGTTG
CCATCGTTGGGCCCCCGGTTCGATTCCGGGCTTGCGCAAAGGTAGAGAGCAGCAGTTG
GTTTCAGAGCTAGGCCAGCATGAGGATCACCCATGCCTGCAGGGCCTAGCAAGTTGAA
ATAAGGCTAGTCCGTTATCAACTTGGCCAGCATGAGGATCACCCATGCCTGCAGGGCC
AAGTGGCACCGAGTCGGTGCTTTTTTT
SEQ ID NO: 5 is an exemplary DNA sequence encoding a dgUtnNT2-mU6-hU6-dgFst-tRNA-
dgEef1a2 multiplex sgRNAs.
AAAAAAAGCACCAGCCGGGAATCGAACCCGGGTCTGTACCGTGGCAGGGTACTATTCT
ACCACTAGACCACTGGTGCTTTGTTGCACCGACTCGGTGCCACTTGGCCCTGCAGGCAT
GGGTGATCCTCATGCTGGCCAAGTTGATAACGGACTAGCCTTATTTCAACTTGCTAGGC
CCTGCAGGCATGGGTGATCCTCATGCTGGCCTAGCTCTGAAACGTCGTGCGTGCTGGC
AAACAAGGCTTTTCTCCAAGGGATATTTATAGTCTCAAAACACACAATTACTTTACAGT
TAGGGTGAGTTTCCTTTTGTGCTGTTTTTTAAAATAATAATTTAGTATTTGTATCTCTTA
TAGAAATCCAAGCCTATCATGTAAAATGTAGCTAGTATTAAAAAGAACAGATTATCTG
TCTTTTATCGCACATTAAGCCTCTATAGTTACTAGGAAATATTATATGCAAATTAACCG
GGGCAGGGGAGTAGCCGAGCTTCTCCCACAAGTCTGTGCGAGGGGGCCGGCGCGGGC
CTAGAGATGGCGGCGTCGGATCGAGGGCCTATTTCCCATGATTCCTTCATATTTGCATA
TACGATACAAGGCTGTTAGAGAGATAATTGGAATTAATTTGACTGTAAACACAAAGAT
ATTAGTACAAAATACGTGACGTAGAAAGTAATAATTTCTTGGGTAGTTTGCAGTTTTAA
AATTATGTTTTAAAATGGACTATCATATGCTTACCGTAACTTGAAAGTATTTCGATTTC
TTGGCTTTATATATCTTGTGGAAAGGACGAAACACCGTGCCCCTCCTTTCCGTTTCAGA
GCTAGGCCAGCATGAGGATCACCCATGCCTGCAGGGCCTAGCAAGTTGAAATAAGGCT
AGTCCGTTATCAACTTGGCCAGCATGAGGATCACCCATGCCTGCAGGGCCAAGTGGCA
CCGAGTCGGTGCAACAAAGCGCAAGTGGTTTAGTGGTAAAATCCAACGTTGCCATCGT
TGGGCCCCCGGTTCGATTCCGGGCTTGCGCACAAAGCGGCAGGAGGTTTCAGAGCTAG
GCCAGCATGAGGATCACCCATGCCTGCAGGGCCTAGCAAGTTGAAATAAGGCTAGTCC
GTTATCAACTTGGCCAGCATGAGGATCACCCATGCCTGCAGGGCCAAGTGGCACCGAG
TCGGTGCTTTTTTT
SEQ ID NO: 6 is an exemplary DNA sequence encoding a dgFst/dgEef1a2/dgUtnNT2/dgUtrnT2
multiplex sgRNAs.
AAAAAAAGCACCGACTCGGTGCCACTTGGCCCTGCAGGCATGGGTGATCCTCATGCTG
GCCAAGTTGATAACGGACTAGCCTTATTTCAACTTGCTAGGCCCTGCAGGCATGGGTG
ATCCTCATGCTGGCCTAGCTCTGAAACTGCCCTTTATTCAATGCACCAGCCGGGAATCG
AACCCGGGTCTGTACCGTGGCAGGGTACTATTCTACCACTAGACCACTGGTGCTTTGTT
GCACCGACTCGGTGCCACTTGGCCCTGCAGGCATGGGTGATCCTCATGCTGGCCAAGT
TGATAACGGACTAGCCTTATTTCAACTTGCTAGGCCCTGCAGGCATGGGTGATCCTCAT
GCTGGCCTAGCTCTGAAACGTCGTGCGTGCTGGCAAACAAGGCTTTTCTCCAAGGGAT
ATTTATAGTCTCAAAACACACAATTACTTTACAGTTAGGGTGAGTTTCCTTTTGTGCTG
TTTTTTAAAATAATAATTTAGTATTTGTATCTCTTATAGAAATCCAAGCCTATCATGTA
AAATGTAGCTAGTATTAAAAAGAACAGATTATCTGTCTTTTATCGCACATTAAGCCTCT
ATAGTTACTAGGAAATATTATATGCAAATTAACCGGGGCAGGGGAGTAGCCGAGCTTC
TCCCACAAGTCTGTGCGAGGGGGCCGGCGCGGGCCTAGAGATGGCGGCGTCGGATCG
AGGGCCTATTTCCCATGATTCCTTCATATTTGCATATACGATACAAGGCTGTTAGAGAG
ATAATTGGAATTAATTTGACTGTAAACACAAAGATATTAGTACAAAATACGTGACGTA
GAAAGTAATAATTTCTTGGGTAGTTTGCAGTTTTAAAATTATGTTTTAAAATGGACTAT
CATATGCTTACCGTAACTTGAAAGTATTTCGATTTCTTGGCTTTATATATCTTGTGGAA
AGGACGAAACACCGTGCCCCTCCTTTCCGTTTCAGAGCTAGGCCAGCATGAGGATCAC
CCATGCCTGCAGGGCCTAGCAAGTTGAAATAAGGCTAGTCCGTTATCAACTTGGCCAG
CATGAGGATCACCCATGCCTGCAGGGCCAAGTGGCACCGAGTCGGTGCAACAAAGCG
CAAGTGGTTTAGTGGTAAAATCCAACGTTGCCATCGTTGGGCCCCCGGTTCGATTCCGG
GCTTGCGCACAAAGCGGCAGGAGGTTTCAGAGCTAGGCCAGCATGAGGATCACCCAT
GCCTGCAGGGCCTAGCAAGTTGAAATAAGGCTAGTCCGTTATCAACTTGGCCAGCATG
AGGATCACCCATGCCTGCAGGGCCAAGTGGCACCGAGTCGGTGCTTTTTTT
SEQ ID NO 7: is an exemplary DNA sequence encoding a modified tracrRNA.
ggaaccattcaaaacagcatagcaagttaaaataaggctagtccgttatcaacttgaaaaagtggcaccgagtcggtgcgggagcGGCCA
GCATGAGGATCACCCATGCCTGCAGGGCCgccacgagcgGGGCCAACATGAGGATCACCCA
TGTCTGCAGGGCCCcgctcgtgttccc
SEQ ID NO: 8 is an exemplary DNA sequence encoding crUT2.
TTGAATAAAGGGCAGTTTTAGAGCTATGCTGTTTTGTTTTTTT
SEQ ID NO: 9 is an exemplary DNA sequence encoding crUT16.
GAGAGCAGCAGTTGGTTTTAGAGCTATGCTGTTTTGTTTTTTT
SEQ ID NO: 10 is an exemplary DNA sequence encoding dgFST.
CAAAGCGGCAGGAGGTTTCAGAGCTAGGCCAGCATGAGGATCACCCATGCCTGCAGG
GCCTAGCAAGTTGAAATAAGGCTAGTCCGTTATCAACTTGGCCAGCATGAGGATCACC
CATGCCTGCAGGGCCAAGTGGCACCGAGTCGGTGCTTTTTTT
SEQ ID NO: 11 is an exemplary DNA sequence encoding dgEef1a2.
TGCCCCTCCTTTCCGTTTCAGAGCTAGGCCAGCATGAGGATCACCCATGCCTGCAGGGC
CTAGCAAGTTGAAATAAGGCTAGTCCGTTATCAACTTGGCCAGCATGAGGATCACCCA
TGCCTGCAGGGCCAAGTGGCACCGAGTCGGTGCTTTTTTT
SEQ ID NO: 12 is an exemplary DNA sequence encoding dgUtrnNT2.
CCAGCACGCACGACGTTTCAGAGCTAGGCCAGCATGAGGATCACCCATGCCTGCAGGG
CCTAGCAAGTTGAAATAAGGCTAGTCCGTTATCAACTTGGCCAGCATGAGGATCACCC
ATGCCTGCAGGGCCAAGTGGCACCGAGTCGGTGCTTTTTTT
SEQ ID NO: 13 is an exemplary DNA sequence encoding dgUtrn.
TTGAATAAAGGGCAGTTTCAGAGCTAGGCCAGCATGAGGATCACCCATGCCTGCAGGG
CCTAGCAAGTTGAAATAAGGCTAGTCCGTTATCAACTTGGCCAGCATGAGGATCACCC
ATGCCTGCAGGGCCAAGTGGCACCGAGTCGGTGCTTTTTTT
SEQ ID NO: 14 is an exemplary DNA sequence encoding dgUtrnT2.
TTGAATAAAGGGCAGTTTCAGAGCTAGGCCAGCATGAGGATCACCCATGCCTGCAGGG
CCTAGCAAGTTGAAATAAGGCTAGTCCGTTATCAACTTGGCCAGCATGAGGATCACCC
ATGCCTGCAGGGCCAAGTGGCACCGAGTCGGTGCTTTTTTT
SEQ ID NO: 15 is an exemplary DNA sequence encoding dgUtrnT16.
GAGAGCAGCAGTTGGTTTCAGAGCTAGGCCAGCATGAGGATCACCCATGCCTGCAGGG
CCTAGCAAGTTGAAATAAGGCTAGTCCGTTATCAACTTGGCCAGCATGAGGATCACCC
ATGCCTGCAGGGCCAAGTGGCACCGAGTCGGTGCTTTTTTT
SEQ ID NO: 16 is an exemplary DNA sequence encoding a native MS2-binding loop
ggccaacatgaggatcacccatgtctgcagggcc
SEQ ID NO: 17 is an exemplary DNA sequence encoding a modified MS2-binding loop
tgctgaacatgaggatcacccatgtctgcagcagca
SEQ ID NO: 18 is an exemplary DNA sequence encoding a modified MS2-binding loop
gggccaacatgaggatcacccatgtctgcagggccc
SEQ ID NO: 19 is an exemplary DNA sequence encoding a modified MS2-binding loop
ggccagcatgaggatcacccatgcctgcagggcc
SEQ ID NO: 20 is an exemplary DNA sequence encoding a Saccharomyces cerevisiae pre-tRNA.
AACAAAGCGCAAGTGGTTTAGTGGTAAAATCCAACGTTGCCATCGTTGGGCCCCCGGT
TCGATTCCGGGCTTGCGCACGAAAT
SEQ ID NO: 21 is an exemplary DNA sequence encoding a Zea mays pre-tRNA
AACAAAGCACCAGTGGTCTAGTGGTAGAATAGTACCCTGCCACGGTACAGACCCGGGT
TCGATTCCCGGCTGGTGCA
SEQ ID NO: 22 is an exemplary DNA sequence encoding a hammerhead RNA.
GGCCGGCATGGTCCCAGCCTCCTCGCTGGCGCCGGCTGGGCAACATGCTTCGGCATGG
CGAATGGGACTGCTGGCTGATGAGTCCGTGAGGACGAAACGAGTAAGCTCGTC
SEQ ID NO: 23 is an exemplary DNA sequence encoding proximal promoter of human EEF1a2.
GGCCCGGTCTTTGGCTTGGCATCCTGACCCCATATGAGCATCAGCTACAAGGCGCTGA
GGTGCAGCGGGGTGGGGCGCTGGGCGGGGGGGCCTGGGTCTGTCTGGATCTGACTCGC
CCTTGGCTGGCGCTGTTTCCCAGCAGCAGCCGGAGGTCGGCGCACCCGGAGGGGAGGG
TCCCTGGAAGATGTCAGTGGGTCTGGGAGCGGGCTTCCGGCGTTCCCTGCACCGTGGG
AGACCAGCCTCTCAGGGGGAGGGTGGTTCTGCGCTGGATCCTCGGGGCCTGTCATGGT
GCGCCCAGGAGGGCAGGCACGTGAGGACAGGGACTGGAAACCAGCAGATTTCCACCC
TGAGGCCTGCACCCCCGGGCCTCATTAGGGAGAGCCCCTCAGAGCCGGGCTTCGTTGG
TTCTGGGGCGTCCCCCATGAGCAGGGCCGGGGAGGGGCCGGTAGACCCAGGCTCGTCT
CCCAGGCTGCAGCCCACCTGCTCCCCTCCCCCGCCTGCCGGCTCCGGTCCTCGGCGTCT
GCCCTGTCCCCGGGGACCGCTTTTCGCGGCTCAAGCGTGTTCCTGCCCTGAGCCGGCTC
TCGCCCCGTCTCCCGGGCCCGCCGCGCTCTCCCCGCGCCGTCTCCGTCCCGGTCCCTCC
CTCCCGCCGCCTCCCTGCCCTGCCCCCCGCCCCGCCCCCGCCCGCGGCGCGTTTCTCCC
CCGCCTCCCGCGTCCGTCTTTGCAGCCCGCGCCTCCCGCATCGCCTCGCGTCCCCGTGG
CGCCCGCCCGCGCGCGTCCGCGCCCCGCCCCCTCCCGCGCGGTTCCGCATTGGCGTGCT
GCAGGGCGCGGTGCACTGCGCCGCCACCGTCAATAGGTGGACCCCCTCCCGGAGATAA
AACCGCCGGCGCCGGCGCCGCCAGTC
SEQ ID NO: 24 is an exemplary DNA sequence encoding proximal promoter of human Fst.
GGAGCCGAGGAGACTGAGAGACAGACAGAGGCACACAGGACAGAAACTGGGGAGTC
TCCAGGCGGGAGAGGAAGGGGGGGCCAGACCGCCTACGTCGGCGCCCCCGCTCCGGG
CTCCGACTCCAGACGCCGCGAAGTGAAAGGGGAGAAAAGAAAGGGAGAGGGCGAGG
CTGTGCCGCGGGGAGACCGGGCCTGAGGTGTTAAACATTTTTGTTTGCTTCCGACTAGT
CCAGACGAAGGGCCGCGTCTCGGTAGCGCTCTGCCAGGGTGGAAGGTGCCGGGGCCG
GGGTTCCTAGCAACACCTCTGGGCTGGGGGTGGCTGCAAAGTCAGGCACTCACAGACC
CAGACACAAAACCTCGCGGGTCCCGCGCCCAGGCTGCGGGTGCCCGGAACCGCCGCG
AGGCCGGCGCGCTCCGACCCGACCCGGGGGGGGATATTTGGGCAGCCCGGGGCTCTTC
GGCCGTTTGCAAAAGTCTCTTTGGAGCGGAGGAGAGGCAGCACGGAGACAAACTCCC
GGGTTCCCCCCGCCACCGCCTCCAGCGCCCCCACCGCGCCCTCCCTCTCACACTCGCGC
GCGCGCGCACACACACTCACACACACACTCACACACACACCCGCCACCCCGGGCGCGC
CGGCGCTGCCGGCGAGCGGCGGCGAGCAGGACTTGAAGTGGGTGTTCTTCCCCACTCC
CCACCCCCGACGCGTAGCCCCCAACCCCCGC
SEQ ID NO: 25 is an exemplary DNA sequence encoding proximal promoter of human Pdx1.
TTAAAAAAAAGAATTTAAAAAAGTCTCTGTGAATGCTTCAGAAGTTACCGTTTACACC
CCAGAAGTACTTGCAGCACATCCACAAGTAAAAACACACAACGAATGCCAGAGTTTCG
TGTGTTTTTTAACCGACATCTTTGTGGCTGTGAACAAACTTCATAAATAAAATAGAATC
AAATGCTTCTGACCTAGAGAGCTGGGTCTGCAAACTTTTTTTTTATCGTATTCCGCAAC
AGTTAAATAAAAAATTAAAAACTCAACATGTCTCCTTGTAAACTACATCAATTAACAA
ACACACTATGTCCATTATCAAATATAATAGAAAAAATATAGGAAAATAGAAAATAGA
AAAATATAGGAAAATAGAAACTTTTAAGCCACGGTGAAAATGTTTCTATAAATGAGTG
GTTCTAATGTTTTCGTGAGCGCCCATTTTGGGGAGCACCGCCAGCTGCCCGTTCAGGAG
TGTGCAGCAAACTCAGCTGAGAGAGAAAATTGGAACAAAAGCAGGTGCTCGCGGGTA
CCTGGGCCTAGCCTCTTAGTGCGGCCAGCCAGGCCAATCACGGCCCCCGGCTGAACCA
CGTGGGGCCCCGCGGAGCCTATGGTGCGGCGGCCGGCCCGCCGGTCCGCGCT
SEQ ID NO: 26 is an exemplary DNA sequence encoding proximal promoter of human klotho.
GTGGCTCTGCAACTTCTGTCAAAAGGGCTCTTTGGCAACAGGAAAAACGTCATGGCTC
CATTGTATTGTAGAGGATGGGAATGGGTGTTCCGGCTAAATTCTCCCTCCCCTTTCCCT
CCACAGCTCAGATGGCAAATGTGCGACCCAGGGACCTCCCGCTCCAGCAGACCTGTGC
GCACAACTTTGCACAGATTACCTGCTAAGTCAGAGCCGAAAGGTAACACAGATGCCAA
AGGATAATAAAGGTGAATGAGATTTACTCAAAATTGGAAACTTGGTGTTTGGTTTTTC
AGGAGAACAATCAACGACTGTGATTTGAAGTTCACCAGGGTATTCTGAGAGATCTAAT
CAAAGATAGAGTGCTGGTTTGAAATTATTAAAAGGTAACAGTAAAAGGGAGAGCAAA
ACCCCAGTCCCAACGCAACCCATAAATCTACTTTGTCTTCCTCGAAAGAGGGGCGCGG
GTGGGCGCGTCTCCCCGCGAGCATCTCACCTAAGGGGGAATCCCTTTCAGCGCACGGC
GAAGTTCCCCCTCGGCTGTCCCACCTGGCAGTCCCTCTAGGATTTCGGCCAGTCCCTAA
TTGGCTCCAGCAATGTCCAGCCGGAGCTTCTTTGGGCCTCCGAGTGGGAGAAAAGTGA
GAGCAGGTGCTTCCCCAGCGGCGCGCTCCGCTAGGGCCCGGCAGGATCCCGCCCCCAA
GTCGGGGAAAGTTGGTCGGCGCCT
SEQ ID NO: 27 is an exemplary DNA sequence encoding proximal promoter of human utrophin.
AACTAGGGGTAAAAAAAAAATCAGCAACGTCAGCAAACTGAGATGGGGTGAGTTGGA
AGGCAGATTGGAATTTATCTCTTAAAAAAATATCACCCTAACTAGAGACCTGTTTTGCC
TAAGGGGACGTGACTCACATTTTCGGATAATCTGAATAAGGGGAATTGTGTCTGCTCG
AGGCATCCATTCTGGTTCGGTCTCCGGACTCCCGGCTCCCGGCACGCACGGTTCACTCT
GGAGCGCGCGCCCCAGGCCAGCCAAGCGCCGAGCCGGGCTGCTGCGGGCTGGGAGGG
CGCGCAGGGCCGGCGCTGATTGACGGGGCGCGCAGTCAGGTGACTTGGGGCGCCAAG
TTCCCGACGCGGTG
SEQ ID NO: 28 is an exemplary DNA sequence encoding proximal promoter of human interleukin
10.
TAAGAAGCTTTCAGCAAGTGCAGACTACTCTTACCCACTTCCCCCAAGCACAGTTGGG
GTGGGGGACAGCTGAAGAGGTGGAAACATGTGCCTGAGAATCCTAATGAAATCGGGG
TAAAGGAGCCTGGAACACATCCTGTGACCCCGCCTGTACTGTAGGAAGCCAGTCTCTG
GAAAGTAAAATGGAAGGGCTGCTTGGGAACTTTGAGGATATTTAGCCCACCCCCTCAT
TTTTACTTGGGGAAACTAAGGCCCAGAGACCTAAGGTGACTGCCTAAGTTAGCAAGGA
GAAGTCTTGGGTATTCATCCCAGGTTGGGGGGACCCAATTATTTCTCAATCCCATTGTA
TTCTGGAATGGGCAATTTGTCCACGTCACTGTGACCTAGGAACACGCGAATGAGAACC
CACAGCTGAGGGCCTCTGCGCACAGAACAGCTGTTCTCCCCAGGAAATCAACTTTTTTT
AATTGAGAAGCTAAAAAATTATTCTAAGAGAGGTAGCCCATCCTAAAAATAGCTGTAA
TGCAGAAGTTCATGTTCAACCAATCATTTTTGCTTACGATGCAAAAATTGAAAACTAA
GTTTATTAGAGAGGTTAGAGAAGGAGGAGCTCTAAGCAGAAAAAATCCTGTGCCGGG
AAACCTTGATTGTGGCTTTTTAATGAATGAAGAGGCCTCCCTGAGCTTACAATATAAA
AGGGGGACAGAGAGGTGAAGGTCTA
SEQ ID NO: 29 is an exemplary DNA sequence encoding proximal promoter of human six2.
GTCGCCCCTCTCCCCCGCCCCGGTGGGCAGACTGCGGGTCTGCGCCGTCCGGGGTTCTG
CGTCGCAGCTGCCGGCCGGAGTCAGCTTCCATAGAGGCCACACGGAACTGCCTGGCGC
TCCTCGGGCTGTGGGACCCGTGGGGTTAAGTCTGAGTCCCCGCCCGGCGAGGAGCAGA
GAGCGCAGAGTTGGGGCGGTACAGGCCGCCAGGCAGCCGGCGGGGCTAGGAGAGGGA
GGAAAGGCGGGATCCTCCGGGAAGTCGATTCTCCGGCGTCCGCCTGCGGCCACTGCCA
AATCTTCCCCATTTCTTTCGTCTACTCCCTCCCCTTTTCCCTCGAGGACCGCTGAGTCCA
GAGTTTCTAGGATGGGGGTGGGGCGCTGTCAGCAGAAAAAGCCAAGTCTTTGGGCGGC
ACCCGAGCACGTCCAAACTCTCCCATCCCACTGGCCTGCGCCGGGGTAGAATGTGCCC
GGTGAACAGAGAGCCTGGGAGGGACGCGGTGACCTGGGGAGAAGGGGAACCCTGTAG
GGTCTGGGCGAGGCTGCAGAGCCCTCTCCTAGCCAAAGCTGCCCAAACTTTCTTCCCCT
GGAGTCTCCTTCCACCCCTCTCCCTCCCCTTCCTCCTGGACACCCCCTTAAACGGTCTCC
GCCTTCCCTTCTCTCCTCTTCTCTCCCCACCTCGATCCACCCCTTTTCGTCTTCGCCCGCT
CCCCCCGCTCTCCTGTCCTCCTCCTCCCTCCCTCTTTGGGCATCCGCCCCGTCAATCTCC
GCCGCCGCCGGCCCCAACCCGGCCCCTCTCCGCCTCCCAGGCTCTCAGAGCGCCCCAG
GCTCCAGTAGAGCCGCCCTCAGTTCTGCGCGGAGCGGGGC
SEQ ID NO: 30 is an exemplary DNA sequence encoding Cas9.
gacaagaagtacagcatcggcctggacatcggcaccaactctgtgggctgggccgtgatcaccgacgagtacaaggtgcccagcaagaaat
tcaaggtgctgggcaacaccgaccggcacagcatcaagaagaacctgatcggagccctgctgttcgacagcggcgaaacagccgaggcca
cccggctgaagagaaccgccagaagaagatacaccagacggaagaaccggatctgctatctgcaagagatcttcagcaacgagatggccaa
ggtggacgacagcttcttccacagactggaagagtccttcctggtggaagaggataagaagcacgagcggcaccccatcttcggcaacatcgt
ggacgaggtggcctaccacgagaagtaccccaccatctaccacctgagaaagaaactggtggacagcaccgacaaggccgacctgcggct
gatctatctggccctggcccacatgatcaagttccggggccacttcctgatcgagggcgacctgaaccccgacaacagcgacgtggacaagc
tgttcatccagctggtgcagacctacaaccagctgttcgaggaaaaccccatcaacgccagcggcgtggacgccaaggccatcctgtctgcca
gactgagcaagagcagacggctggaaaatctgatcgcccagctgcccggcgagaagaagaatggcctgttcggaaacctgattgccctgag
cctgggcctgacccccaacttcaagagcaacttcgacctggccgaggatgccaaactgcagctgagcaaggacacctacgacgacgacctg
gacaacctgctggcccagatcggcgaccagtacgccgacctgtttctggccgccaagaacctgtccgacgccatcctgctgagcgacatcctg
agagtgaacaccgagatcaccaaggcccccctgagcgcctctatgatcaagagatacgacgagcaccaccaggacctgaccctgctgaaag
ctctcgtgcggcagcagctgcctgagaagtacaaagagattttcttcgaccagagcaagaacggctacgccggctacattgacggcggagcc
agccaggaagagttctacaagttcatcaagcccatcctggaaaagatggacggcaccgaggaactgctcgtgaagctgaacagagaggacct
gctgcggaagcagcggaccttcgacaacggcagcatcccccaccagatccacctgggagagctgcacgccattctgcggcggcaggaaga
tttttacccattcctgaaggacaaccgggaaaagatcgagaagatcctgaccttccgcatcccctactacgtgggccctctggccaggggaaac
agcagattcgcctggatgaccagaaagagcgaggaaaccatcaccccctggaacttcgaggaagtggtggacaagggcgcttccgcccaga
gcttcatcgagcggatgaccaacttcgataagaacctgcccaacgagaaggtgctgcccaagcacagcctgctgtacgagtacttcaccgtgt
ataacgagctgaccaaagtgaaatacgtgaccgagggaatgagaaagcccgccttcctgagcggcgagcagaaaaaggccatcgtggacct
gctgttcaagaccaaccggaaagtgaccgtgaagcagctgaaagaggactacttcaagaaaatcgagtgcttcgactccgtggaaatctccgg
cgtggaagatcggttcaacgcctccctgggcacataccacgatctgctgaaaattatcaaggacaaggacttcctggacaatgaggaaaacga
ggacattctggaagatatcgtgctgaccctgacactgtttgaggacagagagatgatcgaggaacggctgaaaacctatgcccacctgttcgac
gacaaagtgatgaagcagctgaagcggcggagatacaccggctggggcaggctgagccggaagctgatcaacggcatccgggacaagca
gtccggcaagacaatcctggatttcctgaagtccgacggcttcgccaacagaaacttcatgcagctgatccacgacgacagcctgacctttaaa
gaggacatccagaaagcccaggtgtccggccagggcgatagcctgcacgagcacattgccaatctggccggcagccccgccattaagaag
ggcatcctgcagacagtgaaggtggtggacgagctcgtgaaagtgatgggccggcacaagcccgagaacatcgtgatcgaaatggccaga
gagaaccagaccacccagaagggacagaagaacagccgcgagagaatgaagcggatcgaagagggcatcaaagagctgggcagccag
atcctgaaagaacaccccgtggaaaacacccagctgcagaacgagaagctgtacctgtactacctgcagaatggggggatatgtacgtgga
ccaggaactggacatcaaccggctgtccgactacgatgtggaccatatcgtgcctcagagctttctgaaggacgactccatcgacaacaaggt
gctgaccagaagcgacaagaaccggggcaagagcgacaacgtgccctccgaagaggtcgtgaagaagatgaagaactactggcggcagc
tgctgaacgccaagctgattacccagagaaagttcgacaatctgaccaaggccgagagaggcggcctgagcgaactggataaggccggctt
catcaagagacagctggtggaaacccggcagatcacaaagcacgtggcacagatcctggactcccggatgaacactaagtacgacgagaat
gacaagctgatccgggaagtgaaagtgatcaccctgaagtccaagctggtgtccgatttccggaaggatttccagttttacaaagtgcgcgaga
tcaacaactaccaccacgcccacgacgcctacctgaacgccgtcgtgggaaccgccctgatcaaaaagtaccctaagctggaaagcgagttc
gtgtacggcgactacaaggtgtacgacgtgcggaagatgatcgccaagagcgagcaggaaatcggcaaggctaccgccaagtacttcttcta
cagcaacatcatgaactttttcaagaccgagattaccctggccaacggcgagatccggaagcggcctctgatcgagacaaacggcgaaaccg
gggagatcgtgtgggataagggccgggattttgccaccgtgcggaaagtgctgagcatgccccaagtgaatatcgtgaaaaagaccgaggtg
cagacaggcggcttcagcaaagagtctatcctgcccaagaggaacagcgataagctgatcgccagaaagaaggactgggaccctaagaagt
acggcggcttcgacagccccaccgtggcctattctgtgctggtggtggccaaagtggaaaagggcaagtccaagaaactgaagagtgtgaaa
gagctgctggggatcaccatcatggaaagaagcagcttcgagaagaatcccatcgactttctggaagccaagggctacaaagaagtgaaaaa
ggacctgatcatcaagctgcctaagtactccctgttcgagctggaaaacggccggaagagaatgctggcctctgccggcgaactgcagaagg
gaaacgaactggccctgccctccaaatatgtgaacttcctgtacctggccagccactatgagaagctgaagggctcccccgaggataatgagc
agaaacagctgtttgtggaacagcacaagcactacctggacgagatcatcgagcagatcagcgagttctccaagagagtgatcctggccgac
gctaatctggacaaagtgctgtccgcctacaacaagcaccgggataagcccatcagagagcaggccgagaatatcatccacctgtttaccctg
accaatctgggagcccctgccgccttcaagtactttgacaccaccatcgaccggaagaggtacaccagcaccaaagaggtgctggacgccac
cctgatccaccagagcatcaccggcctgtacgagacacggatcgacctgtctcagctgggaggcgac
SEQ ID NO: 31 is an exemplary Cas9 amino acid sequence.
MDKKYSIGLDIGTNSVGWAVITDDYKVPSKKFKVLGNTDRHSIKKNLIGALLFDSGEIAEA
TRLKRTARRRYTRRKNRICYLQEIFSNEMAKVDDSFFHRLEESFLVEEDKKHERHPIFGNIV
DEVAYHEKYPTIYHLRKKLVDSTDKADLRLIYLALAHMIKFRGHFLIEGDLNPDNSDVDKL
FIQLVQTYNQLFEENPINASGVDAKAILSARLSKSRRLENLIAQLPGEKKNGLFGNLIALSLG
LTPNFKSNFDLAEDAKLQLSKDTYDDDLDNLLAQIGDQYADLFLAAKNLSDAILLSDILRL
NSEITKAPLSASMIKRYDEHHQDLTLLKALVRQQLPEKYKEIFFDQSKNGYAGYIDGGASQ
EEFYKFIKPILEKMDGTEELLAKLNREDLLRKQRTFDNGSIPHQIHLGELHAILRRQEDFYPF
LKDNREKIEKILTFRIPYYVGPLARGNSRFAWMTRKSEETITPWNFEEVVDKGASAQSFIER
MTNFDKNLPNEKVLPKHSLLYEYFTVYNELTKVKYVTEGMRKPAFLSGEQKKAIVDLLFK
TNRKVTVKQLKEDYFKKIECFDSVEISGVEDRFNASLGTYHDLLKIIKDKDFLDNEENEDIL
EDIVLTLTLFEDREMIEERLKTYAHLFDDKVMKQLKRRRYTGWGRLSRKLINGIRDKQSG
KTILDFLKSDGFANRNFMQLIHDDSLTFKEDIQKAQVSGQGDSLHEHIANLAGSPAIKKGIL
QTVKVVDELVKVMGRHKPENIVIEMARENQTTQKGQKNSRERMKRIEEGIKELGSQILKE
HPVENTQLQNEKLYLYYLQNGRDMYVDQELDINRLSDYDVDHIVPQSFIKDDSIDNKVLT
RSDKNRGKSDNVPSEEVVKKMKNYWRQLLNAKLITQRKFDNLTKAERGGLSELDKAGFI
KRQLVETRQITKHVAQILDSRMNTKYDENDKLIREVKVITLKSKLVSDFRKDFQFYKVREI
NNYHHAHDAYLNAVVGTALIKKYPKLESEFVYGDYKVYDVRKMLAKSEQEIGKATAKYF
FYSNIMNFFKTEITLANGEIRKRPLIETNGETGEIVWDKGRDFATVRKVLSMPQVNIVKKTE
VQTGGFSKESILPKRNSDKLIARKKDWDPKKYGGFDSPTVAYSVLVVAKVEKGKSKKLKS
VKELLGITIMERSSFEKNPIDFLEAKGYKEVRKDLIIKLPKYSLFELENGRKRMLASAGELQ
KGNELALPSKYVNFLYLASHYEKLKGSPEDNEQKQLFVEQHKHYLDEIIEQISEFSKRVILA
DANLDKVLSAYNKHRDKPIREQAENIIHLFTLTNLGAPTAFKYFDTTIDRKRYTSTKEVLD
ATFIHQSITGLYETRIDLSQLGGD
SEQ ID NO: 32 is an exemplary DNA sequence encoding dCas9.
gacaagaagtactccattgggctcgctatcggcacaaacagcgtcggctgggccgtcattacggacgagtacaaggtgccgagcaaaaaatt
caaagttctgggcaataccgatcgccacagcataaagaagaacctcattggcgccctcctgttcgactccggggagacggccgaagccacgc
ggctcaaaagaacagcacggcgcagatatacccgcagaaagaatcggatctgctacctgcaggagatctttagtaatgagatggctaaggtgg
atgactctttcttccataggctggaggagtcctttttggtggaggaggataaaaagcacgagcgccacccaatctttggcaatatcgtggacgag
gtggcgtaccatgaaaagtacccaaccatatatcatctgaggaagaagcttgtagacagtactgataaggctgacttgcggttgatctatctcgc
gctggcgcatatgatcaaatttcggggacacttcctcatcgagggggacctgaacccagacaacagcgatgtcgacaaactctttatccaactgg
ttcagacttacaatcagcttttcgaagagaacccgatcaacgcatccggagttgacgccaaagcaatcctgagcgctaggctgtccaaatcccgg
cggctcgaaaacctcatcgcacagctccctggggagaagaagaacggcctgtttggtaatcttatcgccctgtcactcgggctgacccccaact
ttaaatctaacttcgacctggccgaagatgccaagcttcaactgagcaaagacacctacgatgatgatctcgacaatctgctggcccagatcggc
gaccagtacgcagacctttttttggcggcaaagaacctgtcagacgccattctgctgagtgatattctgcgagtgaacacggagatcaccaaag
ctccgctgagcgctagtatgatcaagcgctatgatgagcaccaccaagacttgactttgctgaaggcccttgtcagacagcaactgcctgagaa
gtacaaggaaattttcttcgatcagtctaaaaatggctacgccggatacattgacggcggagcaagccaggaggaattttacaaatttattaagc
ccatcttggaaaaaatggacggcaccgaggagctgctggtaaagcttaacagagaagatctgttgcgcaaacagcgcactttcgacaatggaag
catcccccaccagattcacctgggcgaactgcacgctatcctcaggcggcaagaggatttctacccctttttgaaagataacagggaaaagatt
gagaaaatcctcacatttcggataccctactatgtaggccccctcgcccggggaaattccagattcgcgtggatgactcgcaaatcagaagaga
ccatcactccctggaacttcgaggaagtcgtggataagggggcctctgcccagtccttcatcgaaaggatgactaactttgataaaaatctgcct
aacgaaaaggtgcttcctaaacactctctgctgtacgagtacttcacagtttataacgagctcaccaaggtcaaatacgtcacagaagggatgag
aaagccagcattcctgtctggagagcagaagaaagctatcgtggacctcctcttcaagacgaaccggaaagttaccgtgaaacagctcaaaga
agactatttcaaaaagattgaatgtttcgactctgttgaaatcagcggagtggaggatcgcttcaacgcatccctgggaacgtatcacgatctcc
tgaaaatcattaaagacaaggacttcctggacaatgaggagaacgaggacattcttgaggacattgtcctcacccttacgttgtttgaagatagg
gagatgattgaagaacgcttgaaaacttacgctcatctcttcgacgacaaagtcatgaaacagctcaagaggcgccgatatacaggatgggggc
ggctgtcaagaaaactgatcaatgggatccgagacaagcagagtggaaagacaatcctggattttcttaagtccgatggatttgccaaccggaa
cttcatgcagttgatccatgatgactctctcacctttaaggaggacatccagaaagcacaagtttctggccagggggacagtcttcacgagcaca
tcgctaatcttgcaggtagcccagctatcaaaaagggaatactgcagaccgttaaggtcgtggatgaactcgtcaaagtaatgggaaggcataa
gcccgagaatatcgttatcgagatggcccgagagaaccaaactacccagaagggacagaagaacagtagggaaaggatgaagaggattga
agagggtataaaagaactggggtcccaaatccttaaggaacacccagttgaaaacacccagcttcagaatgagaagctctacctgtactacctg
cagaacggcagggacatgtacgtggatcaggaactggacatcaatcggctctccgactacgacgtggctgctatcgtgccccagtcttttctca
aagatgattctattgataataaagtgttgacaagatccgataaagctagagggaagagtgataacgtcccctcagaagaagttgtcaagaaaatg
aaaaattattggcggcagctgctgaacgccaaactgatcacacaacggaagttcgataatctgactaaggctgaacgaggtggcctgtctgagt
tggataaagccggcttcatcaaaaggcagcttgttgagacacgccagatcaccaagcacgtggcccaaattctcgattcacgcatgaacacca
agtacgatgaaaatgacaaactgattcgagaggtgaaagttattactctgaagtctaagctggtctcagatttcagaaaggactttcagttttat
aaggtgagagagatcaacaattaccaccatgcgcatgatgcctacctgaatgcagtggtaggcactgcacttatcaaaaaatatcccaagcttga
atctgaatttgtttacggagactataaagtgtacgatgttaggaaaatgatcgcaaagtctgagcaggaaataggcaaggccaccgctaagtact
tcttttacagcaatattatgaattttttcaagaccgagattacactggccaatggagagattcggaagcgaccacttatcgaaacaaacggagaa
acaggagaaatcgtgtgggacaagggtagggatttcgcgacagtccggaaggtcctgtccatgccgcaggtgaacatcgttaaaaagaccgaagt
acagaccggaggcttctccaaggaaagtatcctcccgaaaaggaacagcgacaagctgatcgcacgcaaaaaagattgggaccccaagaaat
acggcggattcgattctcctacagtcgcttacagtgtactggttgtggccaaagtggagaaagggaagtctaaaaaactcaaaagcgtcaagga
actgctgggcatcacaatcatggagcgatcaagcttcgaaaaaaaccccatcgactttctcgaggcgaaaggatataaagaggtcaaaaaaga
cctcatcattaagcttcccaagtactctctctttgagcttgaaaacggccggaaacgaatgctcgctagtgcgggcgagctgcagaaaggtaac
gagctggcactgccctctaaatacgttaatttcttgtatctggccagccactatgaaaagctcaaagggtctcccgaagataatgagcagaagca
gctgttcgtggaacaacacaaacactaccttgatgagatcatcgagcaaataagcgaattctccaaaagagtgatcctcgccgacgctaacctc
gataaggtgctttctgcttacaataagcacagggataagcccatcagggagcaggcagaaaacattatccacttgtttactctgaccaacttggg
cgcgcctgcagccttcaagtacttcgacaccaccatagacagaaagcggtacacctctacaaaggaggtcctggacgccacactgattcatca
gtcaattacggggctctatgaaacaagaatcgacctctctcagctcggtggagac
SEQ ID NO: 33 is an exemplary dCas9 amino acid sequence.
MDKKYSIGLAIGTNSVGWAVITDEYKVPSKKFKVLGNTDRHSIKKNLIGALLFDSGETAEA
TRLKRTARRRYTRRKNRICYLQEIFSNEMAKVDDSFFHRLEESFLVEEDKKHERHPIFGNIV
DEVAYHEKYPTIYHLRKKLVDSTDKADLRLIYLALAHMIKFRGHFLIEGDLNPDNSDVDKL
FIQLVQTYNQLFEENPINASGVDAKAILSARLSKSRRLENLIAQLPGEKKNGLFGNLIALSLG
LTPNFKSNFDLAEDAKLQLSKDTYDDDLDNLLAQIGDQYADLFLAAKNLSDAILLSDILRV
NTEITKAPLSASMIKRYDEHHQDLTLLKALVRQQLPEKYKEIFFDQSKNGYAGYIDGGASQ
EEFYKFIKPILEKMDGTEELLVKLNREDLLRKQRTFDNGSIPHQIHLGELHAILRRQEDFYPF
LKDNREKIEKILTFRIPYYVGPLARGNSRFAWMTRKSEETITPWNFEEVVDKGASAQSFIER
MTNFDKNLPNEKVLPKHSLLYEYFTVYNELTKVKYVTEGMRKPAFLSGEQKKAIVDLLFK
TNRKVTVKQLKEDYFKKIECFDSVEISGVEDRFNASLGTYHDLLKIIKDKDFLDNEENEDIL
EDIVLTLTLFEDREMIEERLKTYAHLFDDKVMKQLKRRRYTGWGRLSRKLINGIRDKQSG
KTILDFLKSDGFANRNFMQLIHDDSLTFKEDIQKAQVSGQGDSLHEHIANLAGSPAIKKGIL
QTVKVVDELVKVMGRHKPENIVIEMARENQTTQKGQKNSRERMKRIEEGIKELGSQILKE
HPVENTQLQNEKLYLYYLQNGRDMYVDQELDINRLSDYDVDAIVPQSFLKDDSIDNKVLT
RSDKNRGKSDNVPSEEVVKKMKNYWRQLLNAKLITQRKFDNLTKAERGGLSELDKAGFI
KRQLVETRQITKHVAQILDSRMNTKYDENDKLIREVKVITLKSKLVSDFRKDFQFYKVREI
NNYHHAHDAYLNAVVGTALIKKYPKLESEFVYGDYKVYDVRKMIAKSEQEIGKATAKYF
FYSNIMNFFKTEITLANGEIRKRPLIETNGETGEIVWDKGRDFATVRKVLSMPQVNIVKKTE
VQTGGFSKESILPKRNSDKLIARKKDWDPKKYGGFDSPTVAYSVLVVAKVEKGKSKKLKS
VKELLGITIMERSSFEKNPIDFLEAKGYKEVKKDLIIKLPKYSLFELENGRKRMLASAGELQ
KGNELALPSKYVNFLYLASHYEKLKGSPEDNEQKQLFVEQHKHYLDEIIEQISEFSKRVILA
DANLDKVLSAYNKHRDKPIREQAENIIHLFTLTNLGAPAAFKYFDTTIDRKRYTSTKEVLD
ATLIHQSITGLYETRIDLSQLGG
SEQ ID NO: 34 is an exemplary DNA sequence encoding a MS2-transcriptional activator fusion
protein.
gcttcaaactttactcagttcgtgctcgtggacaatggtgggacaggggatgtgacagtggctccttctaatttcgctaatggggggcagagtgg
atcagctccaactcacggagccaggcctacaaggtgacatgcagcgtcaggcagtctagtgcccagaagagaaagtataccatcaaggtgga
ggtccccaaagtggctacccagacagtgggcggagtcgaactgcctgtcgccgcttggaggtcctacctgaacatggagctcactatcccaat
tttcgctaccaattctgactgtgaactcatcgtgaaggcaatgcaggggctcctcaaagacggtaatcctatcccttccgccatcgccgctaact
caggtatctacagcgctggaggaggtggaagcggaggaggaggaagcggaggaggaggtagcggacctaagaaaaagaggaaggtggc
ggccgctggatccccttcagggcagatcagcaaccaggccctggctctggcccctagctccgctccagtgctggcccagactatggtgccctc
tagtgctatggtgcctctggcccagccacctgctccagcccctgtgctgaccccaggaccaccccagtcactgagcgctccagtgcccaagtct
acacaggccggcgaggggactctgagtgaagctctgctgcacctgcagttcgacgctgatgaggacctgggagctctgctggggaacagca
ccgatcccggagtgttcacagatctggcctccgtggacaactctgagtttcagcagctgctgaatcagggcgtgtccatgtctcatagtacagcc
gaaccaatgctgatggagtaccccgaagccattacccggctggtgaccggcagccagcggccccccgaccccgctccaactcccctgggaa
ccagcggcctgcctaatgggctgtccggagatgaagacttctcaagcatcgctgatatggactttagtgccctgctgtcacagatttcctctagt
gggcagggaggaggtggaagcggcttcagcgtggacaccagtgccctgctggacctgttcagcccctcggtgaccgtgcccgacatgagcct
gcctgaccttgacagcagcctggccagtatccaagagctcctgtctccccaggagccccccaggcctcccgaggcagagaacagcagcccg
gattcagggaagcagctggtgcactacacagcgcagccgctgttcctgctggaccccggctccgtggacaccgggagcaacgacctgccgg
tgctgtttgagctgggagagggctcctacttctccgaaggggacggcttcgccgaggaccccaccatctccctgctgacaggctcggagcctc
ccaaagccaaggaccccactgtctcctga
SEQ ID NO: 35 is an exemplary MS2-p65-HSF1 amino acid sequence.
MASNFTQFVLVDNGGTGDVTVAPSNFANGVAEWISSNSRSQAYKVTCSVRQSSAQKRKY
TIKVEVPKVATQTVGGVELPVAAWRSYLNMELTIPIFATNSDCELIVKAMQGLLKDGNPIP
SAIAANSGIYSAGGGGSGGGGSGGGGSGPKKKRKVAAAGSPSGQISNQALALAPSSAPVLA
QTMVPSSAMVPLAQPPAPAPVLTPGPPQSLSAPVPKSTQAGEGTLSEALLHLQFDADEDLG
ALLGNSTDPGVFTDLASVDNSEFQQLLNQGVSMSHSTAEPMLMEYPEAITRLVTGSQRPPD
PAPTPLGTSGLPNGLSGDEDESSIADMDFSALLSQISSSGQGGGGSGFSVDTSALLDLFSPSV
TVPDMSLPDLDSSLASIQELLSPQEPPRPPEAENSSPDSGKQLVHYTAQPLFLLDPGSVDTGS
NDLPVLFELGEGSYFSEGDGFAEDPTISLLTGSEPPKAKDPTVS
SEQ ID NO: 36 is an exemplary DNA sequence encoding a 7SK promoter.
TTTAATTCTAGTACTATGCATCGTCTCATTGTCTGCAGTATTTAGCATGCCCCACCCATC
TGCAAGGCATTCTGGATAGTGTCAAAACAGCCGGAAATCAAGTCCGTTTATCTCAAAC
TTTAGCATTTTGGGAATAAATGATATTTGCTATGCTGGTTAAATTAGATTTTAGTTAAA
TTTCCTGCTGAAGCTCTAGTACGATAAGCAACTTGACCTAAGTGTAAAGTTGAGACTTC
CTTCAGGTTTATATAGCTTGTGCGCCGCTTGGGTACCTCG
SEQ ID NO: 37 is an exemplary DNA sequence encoding a Spc5.12 promoter.
CACCGCGGTGGCGGCCGTCCGCCCTCGGCACCATCCTCACGACACCCAAATATGGCGA
CGGGTGAGGAATGGTGGGGAGTTATTTTTAGAGCGGTGAGGAAGGTGGGCAGGCAGC
AGGTGTTGGCGCTCTAAAAATAACTCCCGGGAGTTATTTTTAGAGCGGAGGAATGGTG
GACACCCAAATATGGCGACGGTTCCTCACCCGTCGCCATATTTGGGTGTCCGCCCTCGG
CCGGGGCCGCATTCCTGGGGGCCGGGCGGTGCTCCCGCCCGCCTCGATAAAAGGCTCC
GGGGCCGGCGGCGGCCCACGAGCTACCCGGAGGAGCGGGAGGCGCCAAGCTCTAGAA
CTAGTGGATCCCCC
SEQ ID NO: 38 is an exemplary DNA sequence encoding a Colla2 promoter.
AGATCTGTAAAGAGCCCACGTAGGTGTCCTAAAGTGCTTCCAAACTTGGCAAGGGCGA
GAGAGGGCGGGTGGCTGGGGAGGGCGGAGGTATGCAGACAGGGAGTCAGAGTTCCCC
CTCGAAAGCCTCAAAAGTGTCCACGTCCTCAAAAAGAATGGAACCAATTTAAGAAGCC
CCGTAGCCACGTCCCTCCCCCCTCGGCTCCCTCCCCTGCTCCCCCGCAGTCTCCTCCCA
GCACTGAGTCCCGGGCCCCTAGCCCTAGCCCTCCCATTGGTGGAGACGTTTTTGGAGG
CACCCTCCGGCTGGGGAAACTTTTCCCATATAAATAAGGCAGGTCTGGGCTTTATTATT
TTAGCACCACGGCAGCAGGAGGTTTCGACTAAGTTGGAGGGAACGGTCCACGATTGCA
TGC
SEQ ID NO: 39 is an exemplary DNA sequence encoding an mU6 promoter.
GATCCGACGCCGCCATCTCTAGGCCCGCGCCGGCCCCCTCGCACAGACTTGTGGGAGA
AGCTCGGCTACTCCCCTGCCCCGGTTAATTTGCATATAATATTTCCTAGTAACTATAGA
GGCTTAATGTGCGATAAAAGACAGATAATCTGTTCTTTTTAATACTAGCTACATTTTAC
ATGATAGGCTTGGATTTCTATAAGAGATACAAATACTAAATTATTATTTTAAAAAACA
GCACAAAAGGAAACTCACCCTAACTGTAAAGTAATTGTGTGTTTTGAGACTATAAATA
TCCCTTGGAGAAAAGCCTTGTTTG
SEQ ID NO: 40 is an exemplary DNA sequence encoding an hU6 promoter.
GAGGGCCTATTTCCCATGATTCCTTCATATTTGCATATACGATACAAGGCTGTTAGAGA
GATAATTGGAATTAATTTGACTGTAAACACAAAGATATTAGTACAAAATACGTGACGT
AGAAAGTAATAATTTCTTGGGTAGTTTGCAGTTTTAAAATTATGTTTTAAAATGGACTA
TCATATGCTTACCGTAACTTGAAAGTATTTCGATTTCTTGGCTTTATATATCTTGTGGAA
AGGACGAAACACCG
SEQ ID NO: 41 is an exemplary DNA sequence encoding an H1 promoter.
GAACGCTGACGTCATCAACCCGCTCCAAGGAATCGCGGGCCCAGTGTCACTAGGCGGG
AACACCCAGCGCGCGTGCGCCCTGGCAGGAAGATGGCTGTGAGGGACAGGGGAGTGG
CGCCCTGCAATATTTGCATGTCGCTATGTGTTCTGGGAAATCACCATAAACGTGAAATG
TCTTTGGATTTGGGAATCTTATAAGTTCTGTATGAGACCACTCTTTCCCA
SEQ ID NO: 42 is an exemplary DNA sequence encoding dgMyoD.
AGAGTTGGTAGAGTGTTTCAGAGCTAGGCCAGCATGAGGATCACCCATGCCTGCAGGG
CCTAGCAAGTTGAAATAAGGCTAGTCCGTTATCAACTTGGCCAGCATGAGGATCACCC
ATGCCTGCAGGGCCAAGTGGCACCGAGTCGGTGCTTTTTTT
SEQ ID NO: 43 is an exemplary DNA sequence encoding dgMef2b.
ACTGAGCATAGCTCGTTTCAGAGCTAGGCCAGCATGAGGATCACCCATGCCTGCAGGG
CCTAGCAAGTTGAAATAAGGCTAGTCCGTTATCAACTTGGCCAGCATGAGGATCACCC
ATGCCTGCAGGGCCAAGTGGCACCGAGTCGGTGCTTTTTTT
SEQ ID NO: 44 is an exemplary DNA sequence encoding dgPax7.
ACACCGGCTGCCGTGTTTCAGAGCTAGGCCAGCATGAGGATCACCCATGCCTGCAGGG
CCTAGCAAGTTGAAATAAGGCTAGTCCGTTATCAACTTGGCCAGCATGAGGATCACCC
ATGCCTGCAGGGCCAAGTGGCACCGAGTCGGTGCTTTTTTT
SEQ ID NO: 45 is an exemplary DNA sequence encoding dgOCT4.
GGGGACCTGCACTGGTTTCAGAGCTAGGCCAGCATGAGGATCACCCATGCCTGCAGGG
CCTAGCAAGTTGAAATAAGGCTAGTCCGTTATCAACTTGGCCAGCATGAGGATCACCC
ATGCCTGCAGGGCCAAGTGGCACCGAGTCGGTGCTTTTTTT
SEQ ID NO: 46 is an exemplary DNA sequence encoding dgSOX2.
CCGGCAGCGAGGCTGTTTCAGAGCTAGGCCAGCATGAGGATCACCCATGCCTGCAGGG
CCTAGCAAGTTGAAATAAGGCTAGTCCGTTATCAACTTGGCCAGCATGAGGATCACCC
ATGCCTGCAGGGCCAAGTGGCACCGAGTCGGTGCTTTTTTT
SEQ ID NO: 47 is an exemplary DNA sequence encoding dgKLF.
ATAGCAACGATGGAGTTTCAGAGCTAGGCCAGCATGAGGATCACCCATGCCTGCAGGG
CCTAGCAAGTTGAAATAAGGCTAGTCCGTTATCAACTTGGCCAGCATGAGGATCACCC
ATGCCTGCAGGGCCAAGTGGCACCGAGTCGGTGCTTTTTTT
SEQ ID NO: 48 is an exemplary DNA sequence encoding dgMYC.
CAAAGCAGAGGGCGGTTTCAGAGCTAGGCCAGCATGAGGATCACCCATGCCTGCAGG
GCCTAGCAAGTTGAAATAAGGCTAGTCCGTTATCAACTTGGCCAGCATGAGGATCACC
CATGCCTGCAGGGCCAAGTGGCACCGAGTCGGTGCTTTTTTT
SEQ ID NO: 49 is an exemplary DNA sequence encoding crUCP1.
GAGTGACGCGCGGCGTTTTAGAGCTATGCTGTTTTGTTTTTTT
SEQ ID NO: 50 is an exemplary DNA sequence encoding crPgc1a.
GCGTTACTTCACTGGTTTTAGAGCTATGCTGTTTTGTTTTTTT
SEQ ID NO: 51 is an exemplary DNA sequence encoding crFST.
CAAAGCGGCAGGAGGTTTTAGAGCTATGCTGTTTTGTTTTTTT
SEQ ID NO: 52 is an exemplary DNA sequence encoding crUtrn.
TTGAATAAAGGGCAGTTTTAGAGCTATGCTGTTTTGTTTTTTT
SEQ ID NO: 53 is an exemplary DNA sequence encoding dgUtrnNT2-mU6-hU6-dgUtrnT2
(“UtrnDual”).
AAAAAAAGCACCAGCCGGGAATCGAACCCGGGTCTGTACCGTGGCAGGGTACTATTCT
ACCACTAGACCACTGGTGCTTTGTTGCACCGACTCGGTGCCACTTGGCCCTGCAGGCAT
GGGTGATCCTCATGCTGGCCAAGTTGATAACGGACTAGCCTTATTTCAACTTGCTAGGC
CCTGCAGGCATGGGTGATCCTCATGCTGGCCTAGCTCTGAAACGTCGTGCGTGCTGGC
AAACAAGGCTTTTCTCCAAGGGATATTTATAGTCTCAAAACACACAATTACTTTACAGT
TAGGGTGAGTTTCCTTTTGTGCTGTTTTTTAAAATAATAATTTAGTATTTGTATCTCTTA
TAGAAATCCAAGCCTATCATGTAAAATGTAGCTAGTATTAAAAAGAACAGATTATCTG
TCTTTTATCGCACATTAAGCCTCTATAGTTACTAGGAAATATTATATGCAAATTAACCG
GGGCAGGGGAGTAGCCGAGCTTCTCCCACAAGTCTGTGCGAGGGGGCCGGCGCGGGC
CTAGAGATGGCGGCGTCGGATCGAGGGCCTATTTCCCATGATTCCTTCATATTTGCATA
TACGATACAAGGCTGTTAGAGAGATAATTGGAATTAATTTGACTGTAAACACAAAGAT
ATTAGTACAAAATACGTGACGTAGAAAGTAATAATTTCTTGGGTAGTTTGCAGTTTTAA
AATTATGTTTTAAAATGGACTATCATATGCTTACCGTAACTTGAAAGTATTTCGATTTC
TTGGCTTTATATATCTTGTGGAAAGGACGAAACACCGTTGAATAAAGGGCAGTTTCAG
AGCTAGGCCAGCATGAGGATCACCCATGCCTGCAGGGCCTAGCAAGTTGAAATAAGG
CTAGTCCGTTATCAACTTGGCCAGCATGAGGATCACCCATGCCTGCAGGGCCAAGTGG
CACCGAGTCGGTGCTTTTTTT
SEQ ID NO: 54 is an exemplary DNA sequence encoding dgUtrnNT2-mU6-hU6-dgEef1a2
(“UtrnNT2-Eef1a2”).
AAAAAAAGCACCAGCCGGGAATCGAACCCGGGTCTGTACCGTGGCAGGGTACTATTCT
ACCACTAGACCACTGGTGCTTTGTTGCACCGACTCGGTGCCACTTGGCCCTGCAGGCAT
GGGTGATCCTCATGCTGGCCAAGTTGATAACGGACTAGCCTTATTTCAACTTGCTAGGC
CCTGCAGGCATGGGTGATCCTCATGCTGGCCTAGCTCTGAAACGTCGTGCGTGCTGGC
AAACAAGGCTTTTCTCCAAGGGATATTTATAGTCTCAAAACACACAATTACTTTACAGT
TAGGGTGAGTTTCCTTTTGTGCTGTTTTTTAAAATAATAATTTAGTATTTGTATCTCTTA
TAGAAATCCAAGCCTATCATGTAAAATGTAGCTAGTATTAAAAAGAACAGATTATCTG
TCTTTTATCGCACATTAAGCCTCTATAGTTACTAGGAAATATTATATGCAAATTAACCG
GGGCAGGGGAGTAGCCGAGCTTCTCCCACAAGTCTGTGCGAGGGGGCCGGCGCGGGC
CTAGAGATGGCGGCGTCGGATCGAGGGCCTATTTCCCATGATTCCTTCATATTTGCATA
TACGATACAAGGCTGTTAGAGAGATAATTGGAATTAATTTGACTGTAAACACAAAGAT
ATTAGTACAAAATACGTGACGTAGAAAGTAATAATTTCTTGGGTAGTTTGCAGTTTTAA
AATTATGTTTTAAAATGGACTATCATATGCTTACCGTAACTTGAAAGTATTTCGATTTC
TTGGCTTTATATATCTTGTGGAAAGGACGAAACACCGTGCCCCTCCTTTCCGTTTCAGA
GCTAGGCCAGCATGAGGATCACCCATGCCTGCAGGGCCTAGCAAGTTGAAATAAGGCT
AGTCCGTTATCAACTTGGCCAGCATGAGGATCACCCATGCCTGCAGGGCCAAGTGGCA
CCGAGTCGGTGCTTTTTTT
SEQ ID NO: 55 is an exemplary DNA sequence encoding dgUtrnT2-tRNA-dgUtrnNT2-mU6-
hU6-dgEef1a2 (“UtrnDual-Eef1a2”).
AAAAAAAGCACCGACTCGGTGCCACTTGGCCCTGCAGGCATGGGTGATCCTCATGCTG
GCCAAGTTGATAACGGACTAGCCTTATTTCAACTTGCTAGGCCCTGCAGGCATGGGTG
ATCCTCATGCTGGCCTAGCTCTGAAACTGCCCTTTATTCAATGCACCAGCCGGGAATCG
AACCCGGGTCTGTACCGTGGCAGGGTACTATTCTACCACTAGACCACTGGTGCTTTGTT
GCACCGACTCGGTGCCACTTGGCCCTGCAGGCATGGGTGATCCTCATGCTGGCCAAGT
TGATAACGGACTAGCCTTATTTCAACTTGCTAGGCCCTGCAGGCATGGGTGATCCTCAT
GCTGGCCTAGCTCTGAAACGTCGTGCGTGCTGGCAAACAAGGCTTTTCTCCAAGGGAT
ATTTATAGTCTCAAAACACACAATTACTTTACAGTTAGGGTGAGTTTCCTTTTGTGCTG
TTTTTTAAAATAATAATTTAGTATTTGTATCTCTTATAGAAATCCAAGCCTATCATGTA
AAATGTAGCTAGTATTAAAAAGAACAGATTATCTGTCTTTTATCGCACATTAAGCCTCT
ATAGTTACTAGGAAATATTATATGCAAATTAACCGGGGCAGGGGAGTAGCCGAGCTTC
TCCCACAAGTCTGTGCGAGGGGGCCGGCGCGGGCCTAGAGATGGCGGCGTCGGATCG
AGGGCCTATTTCCCATGATTCCTTCATATTTGCATATACGATACAAGGCTGTTAGAGAG
ATAATTGGAATTAATTTGACTGTAAACACAAAGATATTAGTACAAAATACGTGACGTA
GAAAGTAATAATTTCTTGGGTAGTTTGCAGTTTTAAAATTATGTTTTAAAATGGACTAT
CATATGCTTACCGTAACTTGAAAGTATTTCGATTTCTTGGCTTTATATATCTTGTGGAA
AGGACGAAACACCGTGCCCCTCCTTTCCGTTTCAGAGCTAGGCCAGCATGAGGATCAC
CCATGCCTGCAGGGCCTAGCAAGTTGAAATAAGGCTAGTCCGTTATCAACTTGGCCAG
CATGAGGATCACCCATGCCTGCAGGGCCAAGTGGCACCGAGTCGGTGCTTTTTTT
SEQ ID NO: 56 is the sequence shown in FIG. 5.
ACCTAGTGTGCCTAGAGGGGTGTGACACACATTTTCGGACAATTTGAATAAAGGGCAC
GGTGCGTGCGCGCGGTGACTATTCCAGCTTCTGGCTTCCAGCACGCACGACTGGTTCCG
GGATTCTCGCACCGCGCACCGCACGGAGCCGGCTGCTGCGGGCTGGGAGGGCGCCTA
SEQ ID NO: 57 is the upper band sequence shown in FIG. 13.
GTTTTGAGACTATAAATATCCCTTGGAGAAAAGCCTTGTTTGTTGAATAAAGGGCAGTT
TCAGAGCTAGGCCAGCATGAGGATCACCCATGCCTGCAGGGCCTAGCAAGTTGAAATA
AGGCTAGTCCGTTATCAACTTGGCCAGCATGAGGATCACCCATGCCTGCAGGGCCAAG
TGGCACCGAGTCGGTGCTTTTTTTGAGGGCCTATTTCCCATGATTCCTTCATATTTGCAT
ATACGATACAAGGCTGTTAGAGAGATAATTGGAATTAATTTGACTGTAAACACAAAGA
TATTAGTACAAAATACGTGACGTAGAAAGTAATAATTTCTTGGGTAGTTTGCAGTTTTA
AAATTATGTTTTAAAATGGACTATCATATGCTTACCGTAACTTGAAAGTATTTCGATTT
CTTGGCTTTATATATCTTGTGGAAAGGACGAAACACCGCAAAGCGGCAGGAGGTTTCA
GAGCTAGGCCAGCATGAGGATCACCCATGCCTGCAGGGCCTAGCAAGTTGAAATAAG
GCTAGTCCGTTATCAACTTGGCCAGCATGAGGATCACCCATGCCTGCAGGGCCAAGTG
GCACCGAGTCGGTGCTTTTTTTGTTTTAGAGCTAGCGAATTCGGCTCCGGTGCCCGTCA
GTGGGCAGAGCGCACATCGCCCACAGTC
SEQ ID NO: 58 is the lower band sequence shown in FIG. 13.
GAGACTATAAATATCCCTTGGAGAAAAGCCTTGTTTGTTGAATAAAGGGCAGTTTCAG
AGCTAGGCCAGCATGAGGATCACCCATGCCTGCAGGGCCTAGCAAGTTGAAATAAGG
CTAGTCCGTTATCAACTTGGGCCAACATGAGGATCACCCATGTCTGCAGGGCCCAAGT
GGCACCGAGTCGGTGCTTTTTTTGTTTTAGAGCTAGCGAATTCGGC
SEQ ID NO: 59 is the sequencing product shown in FIG. 22.
ATCAACCCGCTCCAAGGAATCGCGGGCCCAGTGTCACTAGGCGGGAACACCCAGCGC
GCGTGCGCCCTGGCAGGAAGATGGCTGTGAGGGACAGGGGAGTGGCGCCCTGCAATA
TTTGCATGTCGCTATGTGTTCTGGGAAATCACCATAAACGTGAAATGTCTTTGGATTTG
GGAATCTTATAAGTTCTGTATGAGACCACTCTTTCCCAAGAGTTGGTAGAGTGTTTCAG
AGCTAGGCCAGCATGAGGATCACCCATGCCTGCAGGGCCTAGCAAGTTGAAATAAGG
CTAGTCCGTTATCAACTTGGCCAGCATGAGGATCACCCATGCCTGCAGGGCCAAGTGG
CACCGAGTCGGTGCTTTTTTTCTAGCGCGGCCGCAGTATGATACACTTGATGAAGCCGA
ATTCTGCAGATATCCATCACACTGGCGGCCGCTCGAGCATGCATCTAGAGGGCCCAAT
TCGCC
SEQ ID NO: 60 is the sequence product shown in FIG. 52 (top).
GTGGAAAGGACGAAACACCGTTGAATAAAGGGCAGTTTCAGAGCTAGGCCAGCATGA
GGATCACCCATGCCTGCAGGGCCTAGCAAGTTGAAATAAGGCTAGTCCGTTATCAACT
TGGCCAGCATGAGGATCACCCATGCCTGCAGGGCCAAGTGGCACCGAGTCGGTGCAAC
AAAGCACCAGTGGTCTAGTGGTAGAATAGTACCCTGCCACGGTACAGACCCGGGTTCG
ATTCCCGGCTGGTGCACAAAGCGGCAGGAGGTTTCAGAGCTAGGGCCAACATGAGGA
TCACCCATGTCTGCAGGGCCCTAGCAAGTTGAAATAAGGCTAGTCCGTTATCAACTTG
GGCCAACATGAGGATCACCCATGTCTGCAGGGCCCAAGTGGCACCGAGTCGGTGCTTT
TTTTAAGCTTGGCTTGAAT
SEQ ID NO: 61 is the sequence product shown in FIG. 52 (bottom).
ATGCTTACCGTAACTTGAAAGTATTTCGATTTCTTGGCTTTATATATCTTGTGGAAAGG
ACGAAACACCGTTGAATAAAGGGCAGTTTCAGAGCTAGGCCAGCATGAGGATCACCC
ATGCCTGCAGGGCCTAGCAAGTTGAAATAAGGCTAGTCCGTTATCAACTTGGGCCAAC
ATGAGGATCACCCATGTCTGCAGGGCCCAAGTGGCACCGAGTCGGTGCTTTTTTTAAG
CTTGGCTTGAAT

### DETAILED DESCRIPTION

The following explanations of terms and methods are provided to better describe the present disclosure and to guide those of ordinary skill in the art in the practice of the present disclosure. The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements.

Unless explained otherwise, all technical and scientific terms used herein have the same meaning as commonly understood to one of ordinary skill in the art to which this disclosure belongs. Definitions of many common terms in molecular biology may be found in Krebs et al. (eds.), Lewin's genes XII, published by Jones & Bartlett Learning, 2017. All references, including patent applications and patents, and sequences associated with the provided GenBank® Accession numbers (as of Apr. 28, 2021), are herein incorporated by reference in their entireties. Although methods and materials similar or equivalent to those described herein can be used in the practice or testing of the present disclosure, suitable methods and materials are described below. All percentages and ratios are calculated by weight unless otherwise indicated. The term “about” refers to plus or minus 5% of a reference value. For example, “about” 100 refers to 95 to 105.

In case of conflict, the present specification, including explanations of terms, will control.

In addition, the materials, methods, and examples are illustrative only and not intended to be limiting.

In order to facilitate review of the various embodiments of this disclosure, the following explanations of specific terms are provided:

### I. Terms

Administration: To provide or give a subject an agent, such as the disclosed multiplex target gene activation (mTGA) system or portion thereof (such as a nucleic acid encoding a multiplex crRNA or multiplex sgRNA, which may be part of a viral vector, or RNA thereof), by any effective route. Administration can be local or systemic. Exemplary routes of administration include, but are not limited to, oral, injection (such as subcutaneous, intramuscular, intradermal, intraperitoneal, intrahepatic, percutaneous (into the liver), and intravenous), sublingual, rectal, transdermal (for example, topical), intranasal, vaginal, and inhalation routes. In some embodiments, administration is by injection.

Adeno-associated virus (AAV): A small non-enveloped virus that can infect humans and some other primates. It can infect both nondividing and dividing cells. AAV vectors can be used as a gene therapy vector, for example, to deliver a nucleic acid molecule to a target gene using the disclosed mTGA system and related methods. Exemplary AAV vectors that can be used in the methods and compositions provided herein, include AAV1, AAV2, AAV3, AAV4, AAV5, AAV6, AAV7, AAV8, AAV9, AAV10, AAV11, AAV12, AAV-PHP.B, AAV-PHP.eB, and AAV-PHP.S. In some examples, an AAV vector containing, for example, a multiplex crRNA, multiplex sgRNA, Cas9 coding sequence, dCas9 coding sequence, or MS2-transcriptional activator fusion protein coding sequence, has tropism for a specific tissue or cell-type, for example as shown below:

| Tissue | Optimal Serotype |
|---|---|
| CNS | AAV1, AAV2, AAV4, AAV5, AAV8, AAV9, |
| | AAV-PHP.B, AAV-PHP.eB, AAV-PHP.S |
| Heart | AAV1, AAV8, AAV9 |
| Kidney | AAV2 |
| Liver | AAV7, AAV8, AAV9 |
| Lung | AAV4, AAV5, AAV6, AAV9 |
| Pancreas | AAV8 |
| Photoreceptor Cells | AAV2, AAV5, AAV8 |
| RPE (Retinal Pigment | AAV1, AAV2, AAV4, AAV5, AAV8 |
| Epithelium) | |
| Skeletal Muscle | AAV1, AAV6, AAV7, AAV8, AAV9 |

Cas9: An RNA-guided DNA endonuclease enzyme that that participates in the CRISPR-Cas immune defense against prokaryotic viruses. Cas9 has two active cutting sites (HNH and RuvC), one for each strand of the double helix. An exemplary native Cas9 sequence from S. pyogenes is shown in SEQ ID NO: 31.

Catalytically inactive (deactivated or dead) Cas9 (dCas9), which has reduced or abolished endonuclease activity but still binds to dsDNA, is also encompassed by this disclosure. In some examples, a dCas9 includes one or more mutations in the RuvC and HNH nuclease domains, such as one or more of the following point mutations: D10A, E762A, D839A, H840A, N854A, N863A, and D986A (e.g., based on numbering in SEQ ID NO: 31). An exemplary dCas9 sequence with D10A and H840A substitutions is shown in SEQ ID NO: 33. In one example, the dCas9 protein has mutations D10A, H840A, D839A, and N863A (see, e.g., Esvelt et al., Nat. Meth. 10:1116-21, 2013).

In some examples, Cas9 or dCas9 includes a transcriptional activation domain, such as

VP64, P65, MyoD1, HSF1, RTA, SET7/9, or any combination thereof. In other examples, Cas9 or dCas9 does not include a transcriptional activation domain, such as VP64, P65, MyoD1, HSF1, RTA, SET7/9, or any combination thereof.

Cas9 sequences are publicly available. For example, GenBank® Accession Nos. nucleotides 796693 . . . 800799 of CP012045.1 and nucleotides 1100046 . . . 1104152 of CP014139.1 disclose Cas9 nucleic acids, and GenBank® Accession Nos. NP_269215.1, AMA70685.1, and AKP81606.1 disclose Cas9 proteins. In some examples, the Cas9 is a deactivated form of Cas9 (dCas9), such as one that is nuclease deficient (e.g., those shown in GenBank® Accession Nos. AKA60242.1 and KR011748.1). Activatable Cas9 proteins are provided in US Publication No. 2018-0073002-A1.

In certain examples, Cas9 or dCas9 used in the disclosed methods or kits has at least 80% sequence identity, for example at least 85%, at least 90%, at least 95%, at least 98%, or at least 99% sequence identity to such sequences (such as SEQ ID NOS: 31 and 33), and retains the ability to be used in the disclosed methods (e.g., can be used in a mTGA system to increase expression of a target gene).

Complementarity: The ability of a nucleic acid to form hydrogen bond(s) with another nucleic acid sequence by either traditional Watson-Crick base pairing or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, and 10 out of 10 being 50%, 60%, 70%, 80%, 90%, and 100% complementarity, respectively).

Control: A reference standard. In some embodiments, the control is a negative control sample obtained from a healthy subject. In other embodiments, the control is a positive control sample obtained from a subject diagnosed with a disease, for example, a disease associated with low expression of a target gene, such as muscular dystrophy. In still other embodiments, the control is a historical control or standard reference value or range of values (such as a group of samples from subjects with a known diagnosis and/or outcome, or a group of samples that represent baseline or normal values).

A difference between a test sample and a control can be an increase or conversely a decrease. In some examples, expression of a target gene increases relative to a control. The difference can be a qualitative difference or a quantitative difference, for example a statistically significant difference. In some examples, a difference is an increase relative to a control, for example by at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%. In some examples, a difference is a decrease relative to a control, for example by at least about 5%, such as at least about 10%, at least about 20%, at least about 30%, at least about 40%, at least about 50%, at least about 60%, at least about 70%, at least about 80%, at least about 90%, at least about 95%, at least about 98%, at least about 99%, or 100%.

CRISPR/Cas9 system: The CRISPR/Cas system is a prokaryotic immune system that confers resistance to foreign genetic elements, such as plasmids and phages, and provides a form of acquired immunity. CRISPR spacers recognize and cut exogenous genetic elements in a manner analogous to RNAi in eukaryotic organisms. A CRISPR/Cas system can be used to regulate gene expression using the disclosed mTGA system, specifically to activate expression, without cutting double stranded DNA (dsDNA), by delivering a dCas9 protein, dgRNA, or both. Activation of expression of a target gene (or other nucleic acid molecule) can be achieved without cutting dsDNA.

CRISPR RNA (crRNA): A part of the CRISPR/Cas9 system. crRNA is an RNA molecule that hybridizes with tracrRNA to form a unique dual-RNA hybrid structure that binds Cas9 endonuclease and guides it to a target sequence. In addition to a repeat sequence that hybridize with the tracrRNA, the crisprRNA also contains a targeting sequence with complementarity to a target gene. Like dgRNA (described below), crRNA can contain a shortened targeting sequence of about 14 to 15 base pairs, which allows the crRNA to guide wild-type Cas9 to a target sequence, but will not induce a double stranded DNA break. In some examples, the crRNA is an RNA molecule (for example, when expressed in a cell). In some examples, the crRNA is encoded by a DNA molecule (for example, when in a vector, such as a viral vector).

Dead guide RNA (dgRNA): A shortened single guide RNA (sgRNA) that can guide Cas9 to a target sequence, but does not induce double strand DNA breaks. The shortened sgRNAs contain shortened targeting sequences of about 14 to 15 nucleotides, whereas non-dead sgRNAs contain targeting sequences around 20 nucleotides. dgRNAs are further described, for example, in Dahlman et al. (2015) Nat. Biotechnol. 33:1159-1161; Kiani et al. (2015) Nat. Methods, 12:1051-1054; and Hsin-Kai Liao et al. (2017) Cell, 171:1495-1507. In some examples, the dgRNA is an RNA molecule (for example, when expressed in a cell). In some examples, the dgRNA is encoded by a DNA molecule (for example, when in a vector, such as a viral vector).

Effective amount: The amount of an agent (such as the multiplexed sgRNA, multiplexed crRNAs, or mTGA system provided herein) that is sufficient to effect beneficial or desired result. A therapeutically effective amount may vary depending upon one or more of: the subject and disease condition being treated, the weight and age of the subject, the severity of the disease condition, the manner of administration, and the like, which can readily be determined by one of ordinary skill in the art. The beneficial therapeutic effect can include enablement of diagnostic determinations; amelioration of a disease, symptom, disorder, or pathological condition; reducing or preventing the onset of a disease, symptom, disorder, or pathological condition; and generally counteracting a disease, symptom, disorder, or pathological condition. An effective amount can be determined by varying the dosage and measuring the resulting response, such as, for example, expression of a target gene. Effective amounts also can be determined through various in vitro, in vivo or in situ assays.

In one embodiment, an “effective amount” is an amount sufficient to reduce symptoms of a disease, for example, by at least 10%, at least 20%, at least 30%, at least 40%, at least 50%, at least 60%, at least 70%, at least 90%, at least 95%, at least 99%, or 100% (as compared to a suitable control, such as no administration of the therapeutic agent). The term also applies to a dose that will allow sufficient expression of a Cas9 (or dCas9), multiplex crRNA, and/or multiplex sgRNA, to allow for targeting (e.g., modifying expression) of a target gene.

An effective amount encompasses a fractional dose that contributes in combination with previous or subsequent administrations to attaining an effective response. For example, an effective amount of an agent can be administered in a single dose, or in several doses, for example hourly, daily, during a course of treatment lasting several days or weeks. However, the effective amount can depend on the subject being treated, the severity and type of the condition being treated, and the manner of administration. A unit dosage form of the agent can be packaged in an amount, or in multiples of the effective amount, for example, in a vial (e.g., with a pierceable lid), tablet, or other form.

Fusion Protein: A protein that includes at least a portion of the sequence of a full-length first protein (e.g., MS2) and at least a portion of the sequence of a full-length second protein (e.g., a transcriptional activator), where the first and second proteins are different. The two different peptides can be joined directly or indirectly, for example, using a linker (such as a linker of Gly, Ser, or combinations thereof, such as GGGGS). Exemplary fusion proteins include an MS2 domain (e.g., amino acids 1-130 of SEQ ID NO: 35) fused directly or indirectly to one or more transcriptional activation domains, such as one or more of VP64, p65, MyoD1, HSF1, RTA, or SET7/9, such as an MS2-P65-HSF1 fusion protein (e.g. SEQ ID NO: 35, and Konermann et al., Nature, 2015 Jan 29;517(7536):583-8).

Increase or Decrease: A positive or negative change, respectively, in quantity from a reference value. An increase is a positive change, such as an increase at least 25%, at least 50%, at least 75%, at least 100%, at least 200%, at least 300%, at least 400%, or at least 500% as compared to a control value. For example, an increase can be about 25 to 500%, about 25 to 400%, about 25 to 300%, about 25 to 200%, about 25 to 100%, about 25 to 75%, about 25 to 50%, about 50 to 500%, about 75 to 500%, about 100 to 500%, about 200 to 500%, about 300 to 500%, about 400 to 500%, about 50 to 100%, about 50 to 200%, about 50 to 300%, about 50 to 400%, about 50 to 500%, about 100 to 200%, about 100 to 300%, about 100 to 400%, about 100 to 500%, or about 250 to 500%. A decrease is a negative change, such as a decrease of at least 20%, at least 25%, at least 50%, at least 75%, at least 80%, at least 90%, at least 95%, at least 98%, at least 99%, or at least 100% decrease as compared to a control value. For example, a decrease can be about 25 to 100%, about 25 to 98%, about 25 to 95%, about 25 to 90%, about 25 to 80%, about 25 to 75%, about 25 to 50%, about 50 to 100%, about 75 to 100%, about 90 to 100%, about 95 to 100%, about 98 to 100%, about 99 to 100%, about 50 to 75%, about 50 to 80%, about 50 to 90%, about 50 to 95%, about 50 to 98%, about 75 to 80%, about 75 to 90%, about 75 to 95%, or about 75 to 98%.

Inhibiting or treating a disease: “Treatment” refers to a therapeutic intervention that ameliorates a sign or symptom of a disease or pathological condition after infection, when the disease has begun to develop. The term “ameliorating,” with reference to a disease or pathological condition, refers to any observable beneficial effect of the treatment. Inhibiting a disease can include reducing symptoms of the disease. The beneficial effect can be evidenced, for example, by a delayed onset of clinical symptoms of the disease in a subject, a reduction in severity of some or all clinical symptoms of the disease, a slower progression of the disease, an increase in expression of a target gene, an improvement in the overall health or well-being of the subject, or by other parameters that are specific to the particular disease.

A “prophylactic” treatment is a treatment administered to a subject who does not exhibit signs of a disease or exhibits only early signs for the purpose of decreasing the risk of developing pathology. In some embodiments, the disclosed methods are therapeutic and not prophylactic.

Isolated: An “isolated” biological component (e.g., protein, nucleic acid, or cell) has been substantially separated, produced apart from, or purified away from other biological components in the cell or tissue of an organism in which the component occurs, such as other cells, chromosomal and extrachromosomal DNA and RNA, and proteins. Nucleic acids and proteins that have been “isolated” include nucleic acids and proteins purified by standard purification methods. The term also embraces nucleic acids and proteins prepared by recombinant expression in a host cell as well as chemically synthesized nucleic acids and proteins. Isolated vectors containing, for example, the disclosed multiplex crRNA, multiplex sgRNAs, or nucleic acid encoding a protein (such as dCas9, Cas9, or MS2-transcriptional activator fusion protein), or cells containing such vectors, in some examples, are at least 50% pure, such as at least 75%, at least 80%, at least 90%, at least 95%, at least 98%, at least 99%, or 100% pure.

Label: A compound or composition that is conjugated directly or indirectly to another molecule (such as a nucleic acid molecule) to facilitate detection of that molecule. Specific, non-limiting examples of labels include fluorescent and fluorogenic moieties, chromogenic moieties, haptens, affinity tags, and radioactive isotopes. The label can be directly detectable (e.g., optically detectable) or indirectly detectable (for example, via interaction with one or more additional molecules that are in turn detectable).

Liver disease: An acute or chronic disorder of the liver. In some examples, a liver disease is one treated with a liver transplant. Examples of liver diseases that can be treated with the disclosed methods and compositions include, but are not limited to, hepatitis (such as hepatitis A, B or C), fibrosis of the liver, cirrhosis of the liver, alcoholic liver disease, hepatocellular carcinoma, Alagille Syndrome, alpha-1 antitrypsin deficiency (alpha-1), biliary atresia, galactosemia, Gilbert syndrome, hemochromatosis, Lysosomal acid lipase deficiency (LAL-D), non-alcoholic fatty liver disease (NAFLD), primary biliary cholangitis (PBC), primary sclerosing cholangitis (PSC), type I glycogen storage disease (GSD I), blood clotting factor deficiencies (e.g., factors I, II, V, V+VIII, VII, X, XI, or XIII is missing or not working properly), and Wilson disease.

Male-specific bacteriophage 2 (MS2): An RNA virus that includes an RNA operator hairpin that binds a coat protein (i.e., the MS2 domain or MS2 protein; e.g., amino acids 1-130 of SEQ ID NO: 35). MS2-binding loops (i.e., MS2 hairpins or MS2 stem loops; e.g., SEQ ID NO: 16) and MS2 proteins have been incorporated into synergistic activation mediator (SAM) complexes in second-generation CRISPR-Cas9 systems. Modifications of such MS2 hairpin sequences are provided herein (such as SEQ ID NOS: 17-19), which can be incorporated into a sgRNA, for example, a dgRNA, or to modify a tracrRNA. MS2 proteins (e.g., amino acids 1-130 of SEQ ID NO: 35) can be incorporated into fusion proteins to recruit transcription factors.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence (such as a coding sequence of a crRNA, sgRNA, dCas9, ^{Cas9,} or MS2-transcriptional activator fusion protein) if the promoter affects the transcription or expression of the coding sequence. Generally, operably linked DNA sequences are contiguous and, where necessary to join two protein-coding regions, in the same reading frame.

Pharmaceutically acceptable carriers: The pharmaceutically acceptable carriers useful in this invention are conventional. Remington's Pharmaceutical Sciences, by E. W. Martin, Mack Publishing Co., Easton, PA, 15th Edition (1975), describes compositions and formulations suitable for pharmaceutical delivery of the disclosed compositions (e.g., multiplex crRNA, multiplex sgRNA, RNA, vectors, RNP complexes, mTGA system) provided herein.

In general, the nature of the carrier will depend on the particular mode of administration being employed. For instance, parenteral formulations usually include injectable fluids that include pharmaceutically and physiologically acceptable fluids such as water, physiological saline, balanced salt solutions, aqueous dextrose, glycerol or the like as a vehicle. In addition to biologically-neutral carriers, pharmaceutical compositions to be administered can contain minor amounts of non-toxic auxiliary substances, such as wetting or emulsifying agents, preservatives, and pH buffering agents and the like, for example, sodium acetate or sorbitan monolaurate.

Promoter: An array of nucleic acid control sequences that direct transcription of a nucleic acid. A promoter includes necessary nucleic acid sequences near the start site of transcription. A promoter also optionally includes distal enhancer or repressor elements. A “constitutive promoter” is a promoter that is continuously active and is not subject to regulation by external signals or molecules. In contrast, the activity of an “inducible promoter” is regulated by an external signal or molecule (for example, a transcription factor). In some examples, the vectors provided herein include a pol III promoter (e.g., U6 and H1 promoters), a pol II promoter (e.g., the retroviral Rous sarcoma virus (RSV) LTR promoter (optionally with the RSV enhancer), the cytomegalovirus (CMV) promoter (optionally with the CMV enhancer), the SV40 promoter, the Spc5.12 promoter, CW3SL promoter, the dihydrofolate reductase promoter, the β-actin promoter, the phosphoglycerol kinase (PGK) promoter, and the EFla promoter), or combinations thereof.

Recombinant or host cell: A cell that has been genetically altered or is capable of being genetically altered by introduction of an exogenous polynucleotide, such as a recombinant plasmid or vector. Typically, a host cell is a cell in which a vector can be propagated and its nucleic acid expressed. Such cells can be eukaryotic or prokaryotic. The term also includes any progeny of the subject host cell. It is understood that all progeny may not be identical to the parental cell because there may be mutations that occur during replication. However, such progeny are included when the term “host cell” is used.

Regulatory element: A phrase that includes promoters, enhancers, internal ribosomal entry sites (IRES), and other expression control elements (e.g., transcription termination signals, such as polyadenylation signals and poly-U sequences). Such regulatory elements are described, for example, in Goeddel, Gene Expression Technology: Methods In Enzymology 185, Academic Press, San Diego, Calif. (1990). Regulatory elements include those that direct constitutive expression of a nucleotide sequence in many types of host cells and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g., liver, pancreas), or particular cell types (e.g., muscle or liver cells). Regulatory elements may also direct expression in a temporal-dependent manner, such as in a cell-cycle dependent or developmental stage-dependent manner, which may or may not also be tissue or cell-type specific.

Also encompassed by the term “regulatory element” are enhancer elements, such as WPRE; CMV enhancers; the R-U5′ segment in LTR of HTLV-I; SV40 enhancer; and the intron sequence between exons 2 and 3 of rabbit β-globin.

Reporter protein: Any protein whose expression is linked to expression of a gene of interest. Exemplary reporter proteins include fluorescent proteins and chemiluminescent molecules, such as infrared-fluorescent proteins (IFPs), mRFP1, mCherry, mOrange, DsRed, tdTomato, mKO, tagRFP, EGFP, mEGFP, mOrange2, maple, tagRFP-T, firefly luciferase, renilla luciferase, and click beetle luciferase (e.g., US Pat. Pub. No. 2010/0122355). In some examples, the reporter protein is positioned downstream of and in frame with a gene of interest, such that the reporter protein is co-expressed with the gene of interest.

Single Guide RNA (sgRNA): A polynucleotide sequence used to direct a Cas9 or a dCas9 protein to a target nucleic acid sequence. In the endogenous Cas9 system, a trans-activating crRNA (tracrRNA) is an RNA molecule that hybridizes with the repeat sequence of another RNA molecule known as CRISPR RNA (crRNA) to form a unique dual-RNA hybrid structure that binds Cas9 endonuclease and guides it to a target sequence. The crRNA contains a targeting sequence that is complementary to a target gene, thus facilitating binding of the Cas9 complex to the target sequence.

A sgRNA is a synthetic chimera that combines a crRNA and a tracrRNA into a single RNA transcript. The use of sgRNAs simplifies the system while retaining fully functional Cas9-mediated sequence-specific targeting. Changing the targeting sequence within the crRNA portion of the sgRNA allows targeting of any DNA or RNA sequence of interest. (See CRISPR-Cas9 Structures and Mechanisms. Fuguo Jiang and Jennifer A. Doudna, Annual Review of Biophysics, 46:1, 505-529 (2017)).

In some examples, the sgRNA is an RNA molecule (for example, when expressed in a cell).

In some examples, the sgRNA is encoded by a DNA molecule (for example, when in a vector, such as a viral vector). The sgRNA nucleic acids can include modified bases or chemical modifications (e.g., see Latorre et al., Angewandte Chemie 55:3548-50, 2016). In some examples, the sgRNA includes two or more MS2-binding loop sequences, which can be modified from the native MS2-binding loop sequence to increase GC content and/or shorten repetitive content. In some examples, the sgRNA is modified to increase GC content and/or shorten repetitive content. In some examples, the sgRNA is a dead guide RNA (dgRNA). Increasing GC content and/or shortening the repetitive content of the sgRNA can be used to convert the sgRNA into a dgRNA, that is, a guide nucleic acid molecule that can direct a Cas9 or dCas9 protein to a target sequence, but does not induce a DNA double strand break.

Sequence identity/similarity: The similarity between amino acid (or nucleotide) sequences is expressed in terms of the similarity between the sequences, otherwise referred to as sequence identity. Sequence identity is frequently measured in terms of percentage identity (or similarity or homology); the higher the percentage, the more similar the two sequences are.

Methods of alignment of sequences for comparison have been described. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, MD) and on the internet, for use in connection with the sequence analysis programs blastp, blastn, blastx, tblastn and tblastx. A description of how to determine sequence identity using this program is available on the NCBI website on the internet.

Variants of known protein and nucleic acid sequences and those disclosed herein are typically characterized by possession of at least about 80%, at least 90%, at least 95%, at least 96%, at least 97%, at least 98%, or at least 99% sequence identity counted over the full length alignment with the amino acid sequence using the NCBI Blast set to default parameters. When less than the entire sequence is being compared for sequence identity, homologs and variants will typically possess at least 80% sequence identity over short windows of 10-20 amino acids and may possess sequence identities of at least 85% or at least 90% or at least 95%, depending on their similarity to the reference sequence. Methods for determining sequence identity over such short windows are available at the NCBI website on the internet.

In one example, a nucleic acid encoding a multiplex crRNA or multiplex sgRNA has at least 80%, at least 85%, at least 90%, at least 91%, at least 92%, at least 93%, at least 94%, at least 95%, at least 96%, at least 97%, at least 98%, or at least 99% sequence identity to SEQ ID NOS: 1, 2, 3, 4, 5, 6, 53, 54, or 55.

Subject: A vertebrate, such as a human or a non-human mammal. Mammals include, but are not limited to, murines, simians, humans, farm animals, sport animals, and pets. In one embodiment, the subject is a non-human mammalian subject, such as a monkey or other non-human primate, mouse, rat, rabbit, pig, goat, sheep, dog, cat, horse, or cow. In some examples, the subject is a human. In some examples, the subject has a disorder or genetic disease that can be treated using methods provided herein, such as a disorder that results from decreased gene expression. In some examples, the subject is a laboratory animal/organism, such as zebrafish, Xenopus, C. elegans, Drosophila, mouse, rabbit, rat, or primate.

Target gene (or “target”): A gene (or group of genes) that an increase or decrease in expression of the gene product (e.g., protein) is desired, for example, a gene whose activated expression is desired. A gene may be targeted directly or indirectly, so long as there is an effect on the expression of the target gene. In some examples, a targeting sequence (such as a crRNA or sgRNA targeting sequence) has complementarity to the target gene. In some examples, the targeting sequence has complementarity to a promoter and/or regulatory element of the target gene.

Targeting sequence: The portion of a crRNA or sgRNA having complementarity with a target nucleic acid sequence. In some examples, the targeting sequence has complementarity to a promoter or regulatory element of a target gene whose activated expression is desired. In some examples, the targeting sequence is about 14-30 nt and has sufficient complementarity with a target nucleic acid sequence to hybridize with the target sequence and direct sequence-specific binding of a Cas9 or dCas9 to the target nucleic acid sequence. In some embodiments, the degree of complementarity between a targeting sequence and its corresponding target sequence, when optimally aligned using a suitable alignment algorithm, is about or more than about 50%, 60%, 75%, 80%, 85%, 90%, 95%, 97.5%, 98%, 99%, or 100%. In some embodiments, the degree of complementarity is 100%. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net).

Therapeutic agent: Refers to one or more molecules or compounds that confer some beneficial effect upon administration to a subject. The beneficial therapeutic effect can include enablement of diagnostic determinations; amelioration of a disease, symptom, disorder, or pathological condition; reducing or preventing the onset of a disease, symptom, disorder, or pathological condition; and generally counteracting a disease, symptom, disorder, or pathological condition.

Transcriptional activator: A protein or protein domain that increases transcription of a nucleic acid molecule, such as a gene. Such proteins can be used in the methods and mTGA system provided herein, for example, to assist in the recruitment of co-factors and RNA polymerase for the transcription of the target gene. Such proteins and proteins domains can have a DNA binding domain and a domain for activation of transcription. These activators can be introduced into the system through attachment to Cas9, dCas9, sgRNA, tracrRNA, or crRNA. Examples of such activators include VP64, p65, myogenic differentiation 1 (MyoD1), heat shock transcription factor (HSF) 1, RTA, SET7/9, or any combination thereof (such as p65 and HSF1).

Trans-activating crRNA (tracrRNA): An RNA molecule that hybridizes with the repeat sequence of another RNA molecule, known as CRISPR RNA (crRNA), to form a unique dual-RNA hybrid structure that binds Cas9 endonuclease and guides it to a target sequence. Disclosed herein is a modified tracrRNA containing two or more MS2-binding loop sequences modified from the native MS2-binding loop sequence to increase GC content and/or shorten repetitive content. In some examples, the MS2 binding loop sequences facilitate binding by a MS2-transcriptional activator fusion protein. In some examples, the tracrRNA is an RNA molecule (for example, when expressed in a cell). In other examples, the tracrRNA is encoded by a DNA molecule (for example, when in a vector, such as a viral vector).

Transduced, Transformed, and Transfected: A virus or vector “transduces” a cell when it transfers nucleic acid molecules into a cell. A cell is “transformed” or “transfected” by a nucleic acid transduced into the cell when the nucleic acid becomes stably replicated by the cell, either by incorporation of the nucleic acid into the cellular genome or by episomal replication.

These terms encompass all techniques by which a nucleic acid molecule can be introduced into such a cell, including transfection with viral vectors, transformation with plasmid vectors, and introduction of naked DNA by electroporation, lipofection, particle gun acceleration, and other methods in the art. In some examples, the method is a chemical method (e.g., calcium-phosphate transfection), physical method (e.g., electroporation, microinjection, or particle bombardment), fusion (e.g., liposomes), receptor-mediated endocytosis (e.g., DNA-protein complexes or viral envelope/capsid-DNA complexes), and biological infection by viruses, such as recombinant viruses (Wolff, J. A., ed, Gene Therapeutics, Birkhauser, Boston, USA, 1994). Methods for the introduction of nucleic acid molecules into cells are known (e.g., see U.S. Pat. No. 6,110,743). These methods can be used to transduce a cell with the disclosed agents to activate expression.

Transgene: An exogenous gene.

Vector: A nucleic acid molecule into which a foreign nucleic acid molecule can be introduced without disrupting the ability of the vector to replicate and/or integrate in a host cell. Vectors include, but are not limited to, nucleic acid molecules that are single-stranded, double-stranded, or partially double-stranded; nucleic acid molecules that include one or more free ends or no free ends (e.g., circular); nucleic acid molecules that include DNA, RNA, or both; and other varieties of polynucleotides (e.g., LNAs).

A vector can include nucleic acid sequences that permit it to replicate in a host cell, such as an origin of replication. A vector can also include one or more selectable marker genes and other genetic elements. An integrating vector is capable of integrating itself into a host nucleic acid. An expression vector is a vector that contains the necessary regulatory sequences to allow transcription and translation of inserted gene or genes.

One type of vector is a “plasmid,” which refers to a circular double-stranded DNA loop into which additional DNA segments can be inserted, such as by standard molecular cloning techniques. Another type of vector is a viral vector, wherein viral-derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a host cell. In some embodiments, the vector is a lentivirus (such as an integration-deficient lentiviral vector) or adeno-associated viral (AAV) vector.

Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell and, thereby, are replicated along with the host genome.

Certain vectors are capable of directing the expression of genes to which they are operatively-linked. Such vectors are referred to herein as “expression vectors.” Common expression vectors are often in the form of plasmids. Recombinant expression vectors can include a nucleic acid provided herein (such as a multiplex crRNA, multiplex sgRNA, or nucleic acid encoding a protein, such as Cas9, dCas9, or MS2-transcriptional activator fusion protein) in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). It will be appreciated by those skilled in the art that the design of the expression vector can depend on such factors as the choice of the host cell to be transformed, the level of expression desired, etc. A vector can be introduced into host cells to, thereby, produce transcripts, proteins, or peptides, including fusion proteins or peptides, encoded by nucleic acids as described herein.

### II. Overview of Several Embodiments

Duchenne muscular dystrophy (DMD) is caused by the premature mutation of a cytoplasmic protein, dystrophin, leading to progressive muscle degeneration and weakness. A potential treatment strategy is the activation of the utrophin (Utrn) gene (over 10 kbp), a homolog of dystrophin. However, traditional transgene methods are not able to efficiently introduce utrophin into mature muscle due to large gene size and limited AAV capacity. Similar limitations affect the ability to treat other genetic diseases (e.g., see Tables 1 and 2 below).

The CRISPR/Cas9 target gene activation (TGA) system utilizes modified CRISPR/Cas9 machinery and a co-transcriptional complex to 1) rescue levels of gene expression (e.g., restore klotho levels following acute kidney injury or in the mdx model), 2) compensate for genetic defects (e.g., overexpress utrophin to compensate for loss of dystrophin), and 3) alter cell fate by inducing transdifferentiation factors (e.g., generate insulin-producing cells by ectopically expressing Pdx1) (see U.S. application Ser. No. 17/104,372, herein incorporated by reference in its entirety). The TGA system is unmatched in ability to activate genes over 8 kbp as traditional transgene methods are limited by vector capacity. The CRISPR/Cas9-based TGA system uses Cas9 and a modified tracrRNA, sgRNA, or dgRNA containing an MS2-binding aptamer loop to recruit the MS2-p65-HSF1 (MPH) fusion protein to gRNA binding sites within gene promoters for gene activation without cutting the genome. A previous study has showed that the TGA system is able to induce endogenous expression of utrophin, however, the activation level is mild (Liao et al. (2017) Cell, 171(7):1495-1507).

Disclosed herein is a multiplex target gene activation (mTGA) system, which multiplexes CRISPR RNAs (crRNAs) and/or modified single guide RNAs (sgRNAs) to synergistically activate gene expression. It is shown in the examples that activation of utrophin is enhanced when multiple crRNAs and/or sgRNAs are delivered simultaneously without a need to increase total RNA concentration. While several Examples are provided in the context of utrophin activation and the treatment of DMD, this system can be used to activate any other target gene or be used to treat other diseases where activation of a target gene is desired.

### III. Multiplex crRNAs and Multiplex sgRNAs

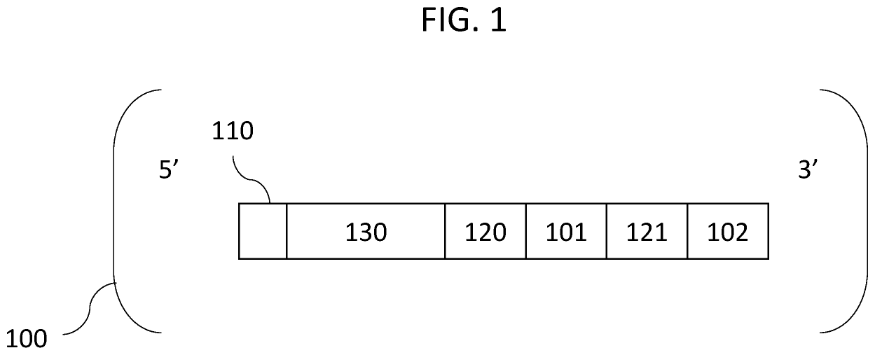

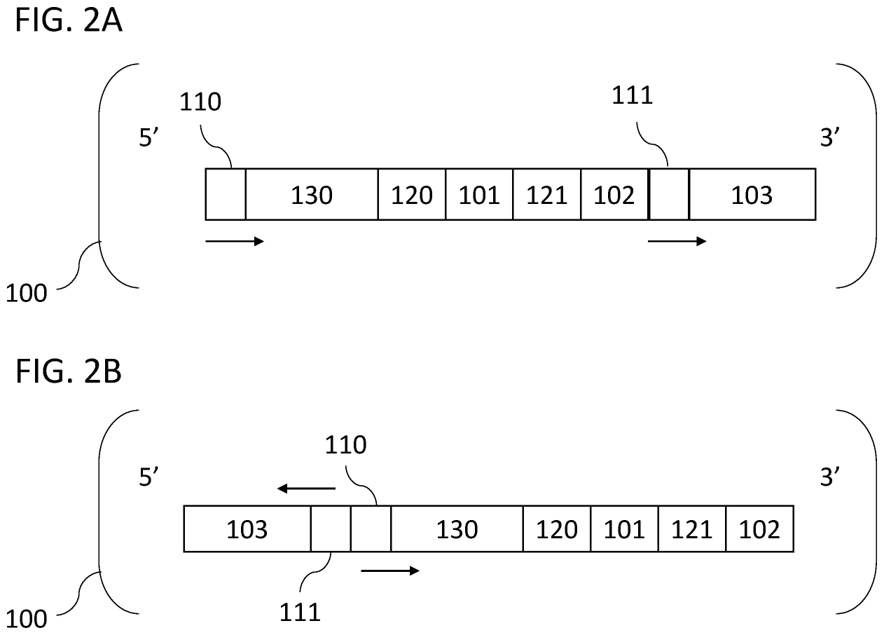

Referring to FIGS. 1-4, provided herein are nucleic acid molecules encoding multiplex CRISPR RNAs (crRNAs) 100 and multiplex single guide RNAs (sgRNAs) 200. One of ordinary skill would recognize that crRNAs and sgRNAs are encoded by DNA when present in a vector (e.g., AAV vector) and that “T” is substituted with “U” when expressed in a cell and transcribed as RNA. Thus, although particular SEQ ID NOs herein show “T” for crRNAs, sgRNAs, or parts thereof, when expressed as RNA, the “T” will become a “U.” In addition, FIGS. 1-4 show the coding sequence (e.g., DNA), as promoters (e.g., 110, 111, 112, 113) are shown, while the corresponding encoded RNA would not include the promoter sequence. Thus, in some examples, 100 and 200 are RNA molecules that do not include a promoter 110, 111, 112, 113. As shown in FIG. 1, in some embodiments, a nucleic acid molecule encoding the multiplex crRNAs 100 encodes multiple crRNAs, for example, two crRNAs (e.g., FIG. 1), three crRNAs (e.g., FIGS. 2A-2B), or more. In some examples, the nucleic acid molecule encoding the multiplex crRNAs 100 includes from 5′ to 3′: a first promoter 110, a nucleic acid molecule encoding a modified trans-activating CRISPR RNA (tracrRNA) 130, a first cleavage site 120, a first nucleic acid molecule encoding a first crRNA 101, a second cleavage site 121, and a second nucleic acid molecule encoding a second erRNA 102.

As shown in FIGS. 2A-2B, in some embodiments, the nucleic acid molecule encoding the multiplex crRNAs 100 further includes a third nucleic acid molecule 103 encoding a third erRNA or a modified single guide RNA (sgRNA) that is operably linked to a second promoter 111. In some examples, the second promoter 111 and third nucleic acid molecule 103 are in forward orientation and are located either i) 3′ of the second nucleic acid molecule encoding the second crRNA 102 (e.g., FIG. 2A) or ii) 5′ of the first promoter (not shown). In other examples, the second promoter 111 and the third nucleic acid molecule 103 are in reverse orientation and located 5′ of the first promoter 110 (e.g., FIG. 2B). Whether the second promoter 111 and third nucleic acid molecule 103 are in “reverse orientation” is determined relative to the orientation of the first promoter 110. Thus, when the second promoter 111 and third nucleic acid 103 are in “reverse orientation,” it means that the sequence of the second promoter and third nucleic acid are read in a direction opposite to the direction of the first promoter 111 (e.g., FIG. 2B).

Since gene targets are independently selected, in some examples the first nucleic acid molecule encoding the first crRNA 101 and the second nucleic acid molecule encoding the second crRNA 102 target different genes, for example, the first crRNA can target utrophin, and the second crRNA can target EEF1a2, Fst, Pdx1, klotho, interleukin 10, or Six2. In other examples, the second erRNA targets utrophin, and the first crRNA targets EEF1a2, Fst. Pdx1, klotho, interleukin 10, or Six2. In a specific, non-limiting example, the first crRNA 101 targets utrophin and the second crRNA 102 targets EEF1a2.

In some embodiments, the first and second crRNAs 101, 102 target the same gene, such as both targeting utrophin. The first and second crRNAs 101, 102 can target the same gene using the same targeting sequence. For example, the first erRNA 101 and the second crRNA 102 can both consist of SEQ ID NO: 8, or SEQ ID NO: 9. The first erRNA 101 and the second crRNA 102 can also target the same gene using different targeting sequences, for example, the first crRNA 101 can consist of SEQ ID NO: 8, while the second crRNA 102 can consist of SEQ ID NO: 9.

In some examples, the first nucleic acid molecule encoding the first crRNA 101, or the second nucleic acid molecule encoding the second crRNA 102, has at least 70%, at least 80%, at least 85%, at least 90%, at least 95%, at least 98%, at least 99%, or 100% sequence identity to SEQ ID NO: 8, 9, 49, 50, 51, or 52, or consists of or includes SEQ ID NO: 8 or SEQ ID NO: 9, 49, 50, 51, or 52. In some examples, the first nucleic acid molecule encoding the first crRNA 101 has at least 95% sequence identity to SEQ ID NO: 8 or SEQ ID NO: 51, or consists of or includes SEQ ID NO: 8 or SEQ ID NO: 51. In further examples, the second nucleic acid molecule encoding the second crRNA 102 has at least 95% sequence identity to SEQ ID NO: 9 or SEQ ID NO: 52, or consists of or includes SEQ ID NO: 9 or SEQ ID NO: 52.

In some examples, the third nucleic acid molecule 103 encodes a modified single guide RNA (sgRNA). The modified sgRNA encodes at least one modified MS2-binding loop sequence. In some examples, the sgRNA encodes two or more modified MS2-binding loop sequences. In some examples, the modified sgRNA is a dgRNA.

In some examples, the modified sgRNA contains a targeting sequence that targets the same gene or sequence as the first crRNA 101, the second crRNA 102, or both. In some examples, the modified sgRNA contains a targeting sequence that targets a different gene or sequence as the first crRNA 101, the second crRNA 102, or both. In a specific, non-limiting example, the first crRNA 101, the second crRNA 102, and the modified sgRNA 103 all target the same gene, such as utrophin. In some examples, the modified sgRNA targets the same gene as the first crRNA 101, the second crRNA 102, or both, but includes a different targeting sequence from the first crRNA 101, the second crRNA 102, or both (e.g., SEQ ID NO: 2). In further examples, the first crRNA 101, the second crRNA 102, and the modified sgRNA all target different genes or sequences, for example, the target of the first crRNA 101, second crRNA 102, and modified sgRNA may be utrophin, EEF1a2, and Fst, respectively. In another non-limiting example, the target of the first crRNA 101, second crRNA 102, and modified sgRNA may be utrophin, EEF1a2, and klotho, respectively.

In some examples, the third nucleic acid molecule 103 encoding the modified sgRNA has at least 70%, at least 809%, at least 85%, at least 90%, at least 95%, at least 98%, at least 99%, or 100% sequence identity to SEQ ID NO: 10, 11, 12, 13, 14, 15, 42, 43, 44, 45, 46, 47, or 48. In some examples, the third nucleic acid molecule 103 encoding the modified sgRNA has at least 95% sequence identity to SEQ ID NO: 10, 11, 12, 13, 14, 15, 42, 43, 44, 45, 46, 47, or 48. In specific, non-limiting examples, the third nucleic acid molecule 103 encoding the modified sgRNA includes or consist of SEQ ID NO: 10, 11, 12, 13, 14, 15, 42, 43, 44, 45, 46, 47, or 48. In another specific, non-limiting example, the third nucleic acid molecule encoding the modified sgRNA 103 has at least 95% sequence identity to SEQ ID NO: 12, or includes or consists of SEQ ID NO: 12.

In a specific, non-limiting example, the first nucleic acid molecule encoding the first crRNA 101 has 90% sequence identity to SEQ ID NO: 8, the second nucleic acid molecule encoding the second crRNA 102 has 90% sequence identity to SEQ ID NO: 9, and the third nucleic acid molecule 103 encoding the modified sgRNA has 90% sequence identity to SEQ ID NO: 12. In another non-limiting example, the first nucleic acid molecule encoding the first crRNA 101 includes or consists of SEQ ID NO: 8, the second nucleic acid molecule encoding the second crRNA 102 includes or consists of SEQ ID NO: 9, and the third nucleic acid molecule 103 encoding the modified sgRNA includes or consists of SEQ ID NO: 12. In a further non-limiting examples, the first nucleic acid molecule encoding the first crRNA 101 has 90% sequence identity to SEQ ID NO: 51, the second nucleic acid molecule encoding the second crRNA 102 has 90% sequence identity to SEQ ID NO: 52. In other examples, the first nucleic acid molecule encoding the first crRNA 101 includes or consists of SEQ ID NO: 51, the second nucleic acid molecule encoding the second crRNA 102 includes or consists of SEQ ID NO: 52.

In some examples, the third nucleic acid molecule 103 encodes a third erRNA. In some examples, the third crRNA contains a targeting sequence that targets the same gene or sequence as the first crRNA, the second erRNA, or both. In some examples, the third crRNA contains a targeting sequence that targets a different gene or sequence as the first crRNA, the second crRNA, or both. In a specific, non-limiting example, the first, second, and third crRNAs are all target the same gene or sequence, such as all targeting utrophin. In some examples, the third crRNA targets the same gene as the first crRNA, the second crRNA, or both, but includes a different targeting sequence from the first crRNA, the second erRNA, or both. In a specific non-limiting example, the first and second erRNA target the same gene or sequence, such as utrophin, and the third crRNA targets a gene or sequence that is different from the first and second crRNAs, such as targeting Fst1 or EEF1a2.

In some examples, the third nucleic acid molecule encoding the third crRNA 103 includes at least 70%, at least 80%, at least 85%, at least 90%, at least 95%, at least 98%, at least 99%, or 100% sequence identity to SEQ ID NO: 8, 9, 51 or 52. In a specific, non-limiting example, the third nucleic acid molecule encoding the third crRNA 103 has at least 95% sequence identity to SEQ ID NO: 8, 9, 51 or 52. In another non-limiting example, the third nucleic acid molecule 103 encoding the third crRNA consist of or includes SEQ ID NO: 8, 9, 51 or 52.

The nucleic acid molecule encoding the modified traerRNA 130 further encodes at least one modified MS2-binding loop. In some examples, the modified traerRNA encodes at least two modified MS2-binding loops. In some examples, the modified tracrRNA comprises one or more of SEQ ID NOS: 17, 18, or 19. In specific, non-limiting examples, the nucleic acid molecule encoding the modified tracrRNA 130 includes at least 70%, at least 80%, at least 85%, at least 90%, at least 95%, at least 989%, at least 99%, or 100% sequence identity to SEQ ID NO: 7. In a specific, non-limiting example, the nucleic acid molecule encoding the modified tracrRNA 103 includes at least 95% sequence identity to SEQ ID NO: 7. In other non-limiting examples, the nucleic acid molecule encoding the modified traerRNA 130 includes or consists of SEQ ID NO: 7.

In some examples, the nucleic acid molecule encoding the multiplex crRNA 100 includes at least 70%, at least 80%, at least 85%, at least 90%, at least 95%, at least 98%, at least 99%, or 100% sequence identity to SEQ ID NO: 1. In a specific example, the nucleic acid molecule encoding the multiplex crRNA 100 has at least 95% sequence identity to SEQ ID NO: 1. In further examples, the nucleic acid molecule encoding the multiplex crRNA 100 includes or consists of SEQ ID NO: 1. In some examples, the nucleic acid molecule encoding the multiplex crRNA 100 has at least 70%, at least 80%, at least 85%, at least 90%, at least 95%, at least 98%, at least 99%, or 100% sequence identity to SEQ ID NO: 2. In specific examples, the nucleic acid molecule encoding the multiplex crRNA 100 has at least 95% sequence identity to SEQ ID NO: 2. In further examples, the nucleic acid molecule encoding the multiplex crRNA 100 includes or consists of SEQ ID NO: 2.

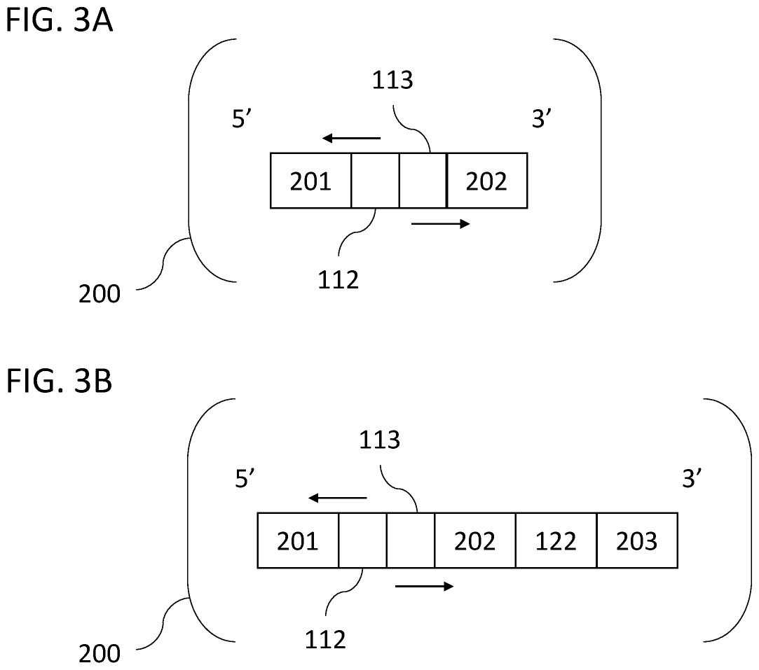

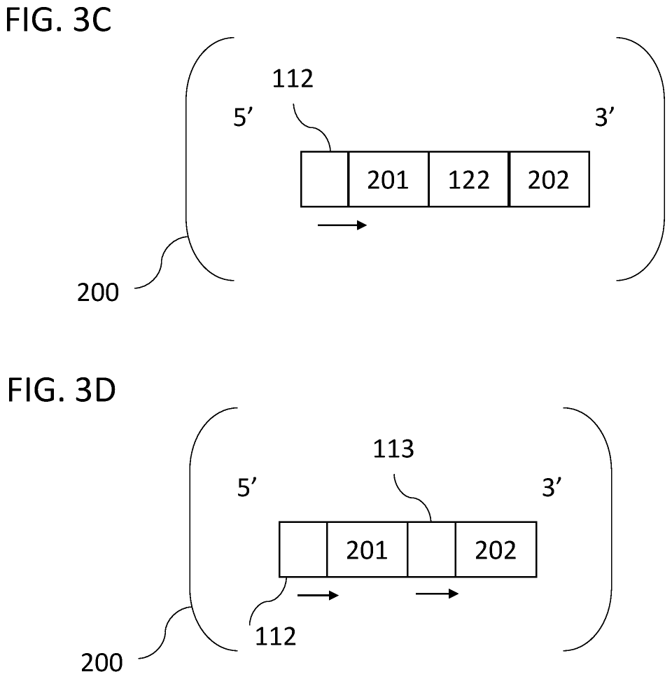

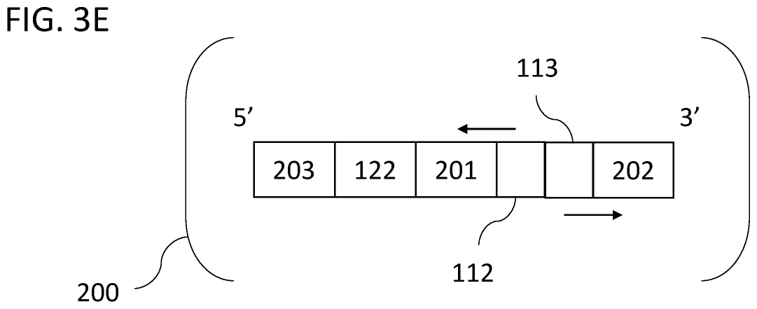

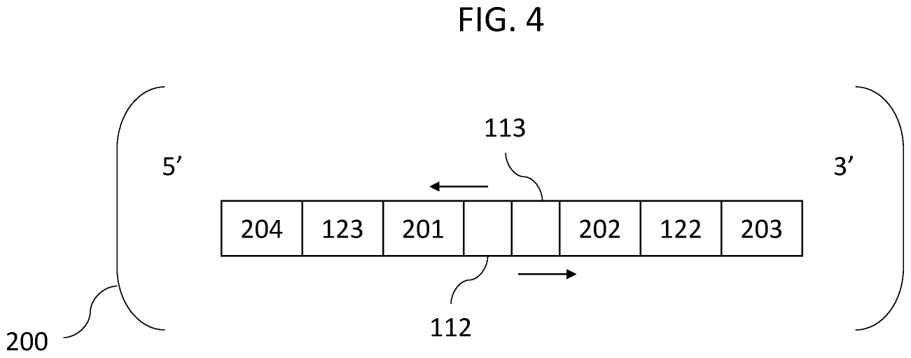

As shown in FIGS. 3-4, also described herein are nucleic acid molecules encoding multiplex sgRNAs 200 containing two or more modified sgRNAs. The modified sgRNA encodes at least one modified MS2-binding loop sequence. In some examples, the modified sgRNA encodes two or more modified MS2-binding loop sequences. In some examples, the modified sgRNA comprises one or more of SEQ ID NOS: 17, 18, or 19. In some examples, the modified sgRNA is a dgRNA.

In some embodiments, the nucleic acid encoding the multiplex sgRNAs 200 encodes two modified sgRNAs (e.g., FIGS. 3A, 3C, and 3D). In some examples, the nucleic acid encoding the multiplex sgRNA 200 includes from 5′ to 3′: a first nucleic acid molecule encoding in reverse orientation a first modified sgRNA 201 operably linked to a first promoter 112, and a second nucleic acid molecule encoding in forward orientation a second modified sgRNA 202 operably linked to a second promoter 113 (see, e.g., FIG. 3A). Whether the first promoter 112 and first modified sgRNA 201 are in “reverse orientation” is determined relative to the orientation of the second promoter 113. Thus, when the first promoter 112 and first modified sgRNA 201 are in “reverse orientation,” it means that the sequence is read in the direction opposite to the direction of the second promoter 113 (e.g. FIGS. 3A, 3B, 3E, and 4). In some examples, the nucleic acid encoding the multiplex sgRNA 200 includes from 5′ to 3′ a first promoter 112 operably linked to: a first nucleic acid molecule encoding a first modified sgRNA 201,a cleavage site 122, and a second nucleic acid molecule 202 (see, e.g., FIG. 3C). In some examples, the nucleic acid encoding the multiplex sgRNA 200 includes from 5′ to 3′ a first promoter 112 operably linked to a first nucleic acid molecule encoding a first modified sgRNA 201,and a second promoter 113 operably linked to a second nucleic acid molecule 202 (see, e.g., FIG. 3D).

In some embodiments, the nucleic acid encoding multiplex sgRNAs 200 encodes three modified sgRNAs (e.g. FIGS. 3B and 3E). The third modified sgRNA 203 is separated from either the first modified sgRNA 201 or the second modified sgRNA 202 by a first cleavage site 122. When the third modified sgRNA 203 is located 3′ of the second modified sgRNA 202,the first cleavage site 122 and the third modified sgRNA 203 are in forward orientation (i.e. the same orientation as the second promoter 113,) and are operably linked to the second promoter 113 (see, e.g., FIG. 3B). Alternatively, the third nucleic acid molecule can be located 5′ of the first modified sgRNA 201 (see, e.g., FIG. 3E). When the third nucleic acid is 5′ of the first modified sgRNA 201,the first cleavage site 122 and the third modified sgRNA 203 are encoded in reverse orientation (i.e., the same orientation as the first promoter 112) and are operably linked to the first promoter 112.

In further examples, the nucleic acid encoding multiplex sgRNAs 200 include four modified sgRNAs (e.g., FIG. 4). When the multiplex sgRNA 200 includes four modified sgRNA coding sequences, the third nucleic acid molecule is located 3′ of the second modified sgRNA 202 and encodes the first cleavage site 122 and the third modified sgRNA 203 in forward orientation (i.e., the same orientation as the second promoter 113,) and is operably linked to the second promoter 113. The fourth nucleic acid is located 5′ of the first modified sgRNA 201 and encodes a second cleavage site 123 and a fourth modified sgRNA 204 in reverse orientation (i.e., the same orientation as the first promoter 112) and is operably linked to the first promoter 112.

In some examples, the nucleic acid sequence of any of the disclosed modified sgRNAs 201, 202, 203, 204, 103 includes at least 70%, at least 80%, at least 85%, at least 90%, at least 95%, at least 98%, at least 99%, or 100% sequence identity to SEQ ID NO: 10, 11, 12, 13, 14, 15, 42, 43, 44, 45, 46, 47, or 48; or consists of or includes SEQ ID NO: 10, 11, 12, 13, 14, 15, 42, 43, 44, 45, 46, 47, or 48.

In some examples, the nucleic acid sequence encoding the first modified sgRNA 201 includes at least 70%, at least 80%, at least 859%, at least 90%, at least 95%, at least 98%, at least 99%, or 100% sequence identity to SEQ ID NO: 10, 12, or 13; or includes or consists of SEQ ID NO: 10, 11, or 13. In some examples, the nucleic acid sequence encoding the second modified sgRNA 202 includes at least 70%, at least 80%, at least 859%, at least 90%, at least 95%, at least 98%, at least 99%, or 100% sequence identity to SEQ ID NO: 10, 11, 13, or 14; or includes or consists of SEQ ID NO: 10, 11, 13, or 14. In some examples, the nucleic acid sequence encoding the third modified sgRNA 203 includes at least 70%, at least 80%, at least 85%, at least 90%, at least 95%, at least 98%, at least 99%, or 100% sequence identity to SEQ ID NO: 10, 11, 14 or 15; or includes or consists of SEQ ID NO: 10, 11, 14 or 15. In some examples, the nucleic acid sequence encoding the fourth modified sgRNA 204 includes at least 70%, at least 80%, at least 85%, at least 90%, at least 95%, at least 98%, at least 99%. or 100% sequence identity to SEQ ID NO: 10, 11, 12, 13, 14 or 15; or includes or consists of SEQ ID NO: 10, 11, 12, 13, 14, or 15.

In a non-limiting example, the nucleic acid molecule encoding the multiplex sgRNA 200 includes at least 70%, at least 80%, at least 85%, at least 90%, at least 95%, at least 98%, at least 99%, or 100% sequence identity to SEQ ID NO: 3, 4, 5, 6, 53, 54, or 55. In some examples, the nucleic acid molecule encoding the multiplex sgRNA 200 includes at least 95% sequence identity to SEQ ID NO: 3, 4, 5, 6, 53, 54, or 55. In further examples, the nucleic acid molecule encoding the multiplex sgRNA 200 includes or consists of SEQ ID NO: 3, 4, 5, 6, 53, 54, or 55.

### Exemplary Targeting Sequences

The disclosed crRNAs 101, 102, 103 and the modified sgRNAs 201, 202, 203, 204, 103 contain a targeting sequence, which facilitates targeting of Cas9 to a sequence of interest. The targeting sequence is independently selected for each crRNA 101, 102, 103 or modified sgRNA 201, 202, 203, 204, 103. Thus, the crRNAs 101, 102, 103 or modified sgRNAs 201, 202, 203, 204, 103 included in the multiplex crRNAs 100 or the multiplex sgRNAs 200 may contain the same targeting sequence, different sequence, or combinations thereof. Thus, each individual crRNA 101, 102, 103 or modified sgRNA 201, 202, 203, 204, 103 may target the same gene, different genes, or combinations thereof.

The targeting sequence has sufficient complementarity to hybridize to a target sequence (e.g., a sequence found within a gene of interest, or within a promoter or regulatory element of a gene of interest). In some examples, the target sequence is targeted in order to modulate expression of a target gene. For example, to activate expression of the target gene. In some examples, the targeting sequence has sufficient complementarity with the target sequence to hybridize with the target sequence and direct sequence-specific binding of a Cas9 or dCas9 to the target sequence.

In some examples, the degree of complementarity between the targeting sequence and its corresponding target sequence, when optimally aligned, is about 50%, about 60%, about 70%, about 80%, about 85%, about 90%, about 95%, about 97.5%, about 98%, about 99%, or 100%. In specific examples, the degree of complementarity between the targeting sequence and its corresponding target sequence is about 90% or more. In specific examples, the degree of complementarity between the targeting sequence and its corresponding target sequence is about 95% or more. Optimal alignment may be determined with the use of any suitable algorithm for aligning sequences. Non-limiting examples include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net).

In some embodiments, the targeting sequence is about 14 to 30 nucleotides in length. For example, about 14, about 15, about 16, about 17, about 18, about 19, about 20, about 21, about 22, about 23, about 24, about 25, about 26, about 27, about 28, about 29, or about 30 nucleotides in length. In further examples, the targeting sequence is about 14 to 28, about 14 to 26, about 14 to 24, about 14 to 22, about 14 to 20, about 14 to 18, about 14 to 17, about 14 to 16, about 14 to 15, about 16 to 30, about 18 to 30, about 20 to 30, about 22 to 30, about 24 to 30, about 26 to 30, about 28 to 30 nucleotides. In specific, non-limiting examples, the targeting sequence is about 14 to 16 nucleotides.

In some examples, the targeting sequence is complementary to a sequence near a transcriptional start site of the target gene, for example, in the promoter region of the target gene. In some examples, the targeting sequence is complementary to a sequence that is within about 10, about 25, about 50, about 60, about 70, about 80, about 90, about 100, about 110, about 120, about 130, about 140, about 150, about 175, about 200, about 300, about 400, or about 500 nucleotides of the transcriptional start site. In further examples, the targeting sequence is complementary to a sequence that is within about 1 to 50, about 1 to 100, about 1 to 150, about 1 to 200, about 1 to 300, about 1 to 400, about 1 to 500, about 10 to 500, about 50 to 500, about 100 to 500, about 150 to 500, about 200 to 500, about 250 to 500, about 300 to 500, about 350 to 500, about 400 to 500, about 10 to 50, about 10 to 100, about 10 to 150, about 10 to 200, about 10 to 250, about 10 to 300, about 10 to 350, about 10 to 400, about 10 to 450, about 25 to 50, about 25 to 100, about 25 to 150, about 25 to 200, about 25 to 250, about 25 to 300, about 25 to 350, about 25 to 400, about 25 to 450, about 50 to 100, about 50 to 150, about 50 to 200, about 50 to 250, about 50 to 300, about 50 to 350, about 50 to 400, about 50 to 450, about 100 to 200, about 100 to 250, about 100 to 300, or about 100 to 400 nucleotides of the transcriptional start site. In a specific, non-limiting example, the targeting sequence is complementary to a sequence that is within about 200 nucleotides of the transcriptional start site.

A targeting sequence can be designed such that multiple genes are targeted. For example, a targeting sequence can be designed to target a sequence that is conserved among a group of gene targets. For example, a target sequence that is conserved, for example, among about 2, about 3, about 4, about 5, about 6, about 7, about 8, about 9, about 10, or more target genes. Thus, the term “target,” as used in connection with a gene includes single gene targets, or multiple gene targets capable of being targeted by a single targeting sequence.

In some embodiments, the gene target is a gene in which decreased expression results in a disease or disorder in a subject, or wherein increased expression can reduce symptoms of a disease or disorder. Thus, activated gene expression is desired. Non-limiting examples of diseases and exemplary gene targets for activation are shown in Table 1 and Table 2 below.

TABLE 1

| Disease | Targets | References |
|---|---|---|
| Acute Kidney Disease | IL-10, Klotho | Liao et al. (2017) Cell, 171(7): 1495-1507 |
| Aging | TERT, Oct4, Sox2, | Bernardes et al. (2012) EMBO Mol Med. (8): 691-704 |
| | Klf4, c-Myc | Ocampo et al. (2016) Cell, 167(7): 1719-1733 |
| Alzheimer's | Neprilysin, | El-Amouri et al. (2008) Am J Pathol., 172(5): 1342-54 |
| disease | NGF, BDNF | Nilsson et al. (2010) J. Cell Mol. Med., 14(4): 741-757 |
| Cancer | Bax, Notch3 | Falke et al. (2003) Nucleic Acids Res., 31(3): e10 |
| | | Rahman et al. (2012) Am J Clin Pathol. 38(4): 535-44 |
| Degenerative motor | HGF, CNTF, | Sun et al. (2002) J Neurosci., 22(15): 6537-48 |
| neuron diseases | Artemin | Schaller et al., (2017) PNAS 114(12): E2486-E2493 |
| Diabetes | Pdx1, MafA, | Liao et al. (2017) Cell, 171(7): 1495-1507 |
| | Ngn3 | Xiao et al., (2018) Cell Stem Cell, 22(1): 78-90 |
| | | Zhou et al. (2008) Nature, 455(7213): 627-32 |
| | | Cavelti-Weder et al. (2017) Curr Protoc Stem Cell Biol. |
| | | 40: 4A.10.1-4A.10.12 |
| Liver fibrosis | Foxa3, Gata4, | Song et al. (2016) Cell Stem Cell, 18(6): 797-808 |
| | HNF1 HNF4a | |
| Muscular | Utrophin, | Liao et al. (2017) Cell, 171(7): 1495-1507 |
| Dystrophy | Follistatin, Klotho | |
| Myocardial disorders | Tbx18 | Kapoor et al. (2013) Nat Biotechnol., 31(1): 54-62 |
| Sickle cell disease | Gamma-Globin | Gräslund et al. (2005) J Biol Chem., 280(5): 3707-14 |

Additional non-limiting examples of gene targets and diseases are shown in Table 2.

TABLE 2

| Gene | NCBI Ref. No. | Exemplary Disease |
|---|---|---|
| EEF1α2 | NG_034083.1 | Muscular Dystrophy |
| Fst | NG_028911.1 | Muscular Dystrophy |
| Pdx1 | NG_008183.1 | Muscular Dystrophy |
| klotho | NG_011485.1 | Muscular Dystrophy |
| utrophin | NG_042293.1 | Muscular Dystrophy |
| interleukin 10 | NG_012088.1 | Muscular Dystrophy |
| Six2 | NC_000002.12 | Muscular Dystrophy |
| TTR | S63185.1 | Amyloid neuropathy |
| APOA1 | NG_012021.1 | Amyloidosis |
| APP | NG_007376.1 | Amyloidosis |
| GSN | NG_012872.1 | Amyloidosis |
| FGA | NG_008832.1 | Amyloidosis |
| LYZ | NG_008195.1 | Amyloidosis |
| KRT18 | NG_008351.1 | Cirrhosis |
| KRT8 | NG_008402.2 | Cirrhosis |
| CIRH1A | NG_008278.1 | Cirrhosis |
| CFTR | NG_016465.4 | Cystic fibrosis |
| MRP7 | AL359813.23 | Cystic fibrosis |
| SLC2A2 | NG_008108.1 | Glycogen storage diseases |
| G6PC | NG_011808.1 | Glycogen storage diseases |
| G6PT1 | NG_013331.1 | Glycogen storage diseases |
| GAA | NG_009822.1 | Glycogen storage diseases |
| LAMP2 | NG_007995.1 | Glycogen storage diseases |
| AGL | NG_012865.1 | Glycogen storage diseases |
| GBE1 | NG_011810.1 | Glycogen storage diseases |
| GYS2 | NG_016167.1 | Glycogen storage diseases |
| PYGL | NG_012796.1 | Glycogen storage diseases |
| PFKM | NG_016199.2 | Glycogen storage diseases |
| HNF1A | NG_011731.2 | Hepatic adenoma, 142331 |
| SCO1 | NG_008228.2 | Hepatic failure, early onset, and |
| | | neurologic disorder |
| LIPC | NG_011465.1 | Hepatic lipase deficiency |
| CTNNB1 | NG_013302.2 | Hepatoblastoma |
| PDGFRL | NG_023332.1 | Hepatoblastoma |
| AXIN1 | NG_012267.1 | Hepatoblastoma |
| TP53 | NG_017013.2 | Hepatoblastoma |
| IGF2R | NG_011785.3 | Hepatoblastoma |
| MET | NG_008996.1 | Hepatoblastoma |
| CASP8 | NG_007497.1 | Hepatoblastoma |
| UMOD | NG_008151.1 | Medullary cystic kidney disease |
| HNF1B | NG_013019.2 | Medullary cystic kidney disease |
| PAH | NG_008690.2 | Phenylketonuria |
| QDPR | NG_008763.1 | Phenylketonuria |
| PTS | NG_008743.1 | Phenylketonuria |
| PKHD1 | NG_008753.1 | Polycystic kidney and hepatic disease |
| PKD1 | NG_008617.1 | Polycystic kidney and hepatic disease |
| PKD2 | NG_008604.1 | Polycystic kidney and hepatic disease |
| PRKCSH | NG_009300.1 | Polycystic kidney and hepatic disease |
| SEC63 | NG_008270.1 | Polycystic kidney and hepatic disease |
| PCSK9 | NG_009061.1 | Liver Disease |
| Hmgcr | NG_011449.1 | Liver Disease |
| SERPINA1 | NG_008290.1 | Liver Disease |
| ApoB | NG_011793.1 | Liver Disease |
| HNF4A | NG_009818.1 | Liver fibrosis/cirrhosis |
| FOXA2 | AF147787.1 | Liver fibrosis/cirrhosis |
| OCT4 | Gene ID: 5460 | Liver fibrosis/cirrhosis |
| FOXA1 | AF147787.1 | Liver fibrosis/cirrhosis |
| FOXA3 | GeneID: 3171 | Liver fibrosis/cirrhosis |
| HNF6 | GeneID: 3175 | Liver fibrosis/cirrhosis |
| GATA4 | NG_008177.2 | Liver fibrosis/cirrhosis |
| HLF | NG_046944.1 | Liver fibrosis/cirrhosis |
| CEBPA | NG_012022.1 | Liver fibrosis/cirrhosis |
| PROX1 | Gene ID: 5629 | Liver fibrosis/cirrhosis |
| AMT | NG_015986.1 | Liver Disease, fibrosis/cirrhosis |
| ADA | NG_007385.1 | Liver Disease, fibrosis/cirrhosis |
| PPOX | NG_012877.2 | Liver Disease, fibrosis/cirrhosis |
| UROD | NG_007122.2 | Liver Disease, fibrosis/cirrhosis |
| HMBS | NG_008093.1 | Liver Disease, fibrosis/cirrhosis |
| ACADVL | NG_007975.1 | Liver Disease, fibrosis/cirrhosis |
| PC | NG_008319.1 | Liver Disease, fibrosis/cirrhosis |
| IVD | NG_011986.2 | Liver Disease, fibrosis/cirrhosis |
| APOA5 | NG_015894.1 | Liver Disease, fibrosis/cirrhosis |
| GALT | NG_009029.2 | Liver Disease, fibrosis/cirrhosis |
| LDLRAP1 | NG_008932.1 | Liver Disease, fibrosis/cirrhosis |
| GCK | NG_008847.2 | Liver Disease, fibrosis/cirrhosis |
| POGLUT1 | NG_034115.1 | Liver Disease, fibrosis/cirrhosis |
| PIK3R1 | NG_012849.2 | Liver Disease, fibrosis/cirrhosis |
| TRIB1 | FJ515869.1 | Liver Disease, fibrosis/cirrhosis |
| TGFB1 | NG_013364.1 | Liver Disease, fibrosis/cirrhosis |
| HAMP | NG_011563.1 | Liver Disease, fibrosis/cirrhosis |
| THPO | NG_012136.1 | Liver Disease, fibrosis/cirrhosis |
| PNPLA3 | NG_008631.1 | Liver Disease, fibrosis/cirrhosis |
| ATP7B | NG_008806.1 | Liver Disease, fibrosis/cirrhosis |
| FAH | NG_012833.1 | Liver Disease, fibrosis/cirrhosis |
| ASL | NG_009288.1 | Liver Disease, fibrosis/cirrhosis |
| HFE | NG_008720.2 | Liver Disease, fibrosis/cirrhosis |
| ALMS1 | NG_011690.1 | Liver Disease, fibrosis/cirrhosis |
| PPARD | NG_012345.1 | Liver Disease, fibrosis/cirrhosis |
| IL6 | NG_011640.1 | Liver Disease, fibrosis/cirrhosis |
| HSD3B7 | NG_012346.1 | Liver Disease, fibrosis/cirrhosis |
| CERS2 | Gene ID: 29956 | Liver Disease, fibrosis/cirrhosis |
| NCOA5 | Gene ID: 57727 | Liver Disease, fibrosis/cirrhosis |
| HEX | Gene ID: 3087 | Liver Disease, fibrosis/cirrhosis |
| HNF6 | Gene ID: 378468 | Liver Disease, fibrosis/cirrhosis |
| SOX17 | NG_028171.1 | Liver Disease, fibrosis/cirrhosis |

Additional examples can be found in U.S. Pat. No. 10,550,372.

In some examples, the crRNA (e.g., 101, 102, 103) or modified sgRNA (e.g., 201, 202, 203, 204, 103) target a gene whose activated expression is desired. For example, targeting one or more genes listed in Table 1 or Table 2. In some examples, the gene target is activated by using a targeting sequence complementary to a promoter or regulatory region of a target gene, for example, one or more genes listed Table 1 or Table 2. In a specific non-limiting example, the crRNA (e.g., 101, 102, 103) or modified sgRNA (e.g., 201, 202, 203, 204, 103) include a targeting sequence complementary to a sequence within the promoter region of EEF1a2, Fst, Pdx1, klotho, utrophin, interleukin 10, Six2, OCT4, SOX2, KLF4, c-MYC, MyoD, Mef2b, or Pax7. In another non-limiting example, the crRNA (e.g., 101, 102, 103) or modified sgRNA (e.g., 201, 202, 203, 204, 103) include a targeting sequence complementary to a sequence within the promoter region of utrophin, EEF1a2, or Fst. In further examples, the crRNA (e.g., 101, 102, 103) or modified sgRNA (e.g., 201, 202, 203, 204, 103) include a targeting sequence complementary to a sequence within the promoter region of utrophin, EEF1a2, or klotho. In some examples, the crRNA (e.g., 101, 102, 103) or modified sgRNA (e.g., 201, 202, 203, 204, 103) include a targeting sequence complementary to a sequence within the promoter region of utrophin. In another specific, non-limiting example, the crRNA (e.g., 101, 102, 103) or modified sgRNA (e.g., 201, 202, 203, 204), 103 include a targeting sequence complementary to a sequence within the promoter region of Foxa3, Gata4, HNF1a, HNF4a.

### Exemplary Modified MS2 Binding Loops

In some embodiments, the modified sgRNA (e.g., 201, 202, 203, 204, 103) or modified tracrRNA (e.g., 130) contain two or more modified MS2 binding loops. The sequence of the modified MS2-binding Joop contains at least two nucleotide changes from the native MS2-binding loop sequence of ggccaacatgaggatcacccatgtcigcagggce (SEQ ID NO: 16), thereby increasing the GC content and/or shortening the repetitive content of the modified MS2-binding loop sequence relative to the native MS2-binding loop sequence. For example, the modified MS2-binding loop sequences can include about 2, about 3, about 4, about 5, about 6, about 7, about 8, about 9, or about 10 nucleotide changes to the native MS2-binding loop sequence ggccaacatgaggatcacccatgtctgcagggcc (SEQ ID NO: 16) that increases the GC content of the native sequence, such as increasing GC content by about 5%, about 10%, about 15%, about 20%, about 25%, about 30%, about 35%, about 40%, about 45%, about 50%, or more. In further examples, there are at least four nucleotide changes. A suitable percent increase includes, for example, about 1 to 5%, about 1 to 8%, about 1 to 10%, about 1 to 12%, about 1 to 15%, about 1 to 20%, about 1 to 30%, about 1 to 40%, about 1 to 50%, about 1 to 60%, about 5 to 10%, about 5 to 20%, about 5 to 30%, about 5 to 40%, about 5 to 50%, about 5 to 60%, about 10 to 20%, about 10 to 30%, about 10 to 40%, about 10 to 50%, about 10 to 60%, about 20 to 30%, about 20 to 40%, about 20 to 50%, about 20 to 60%, about 30 to 40%, about 30 to 50%, about 30 to 60%, about 40 to 50%, about 40 to 60%, or about 50 to 60%. In some examples, the GC content of a nucleic acid molecule is increased by adding “G” and/or “C” nucleotides to the molecule by substituting one or more native “A” to a “G” or substituting one or more native “T” to a “C,” or combinations thereof. In some examples, the modified MS2-binding loop sequences includes about 2 nucleotide changes, thereby increasing GC content of the MS2-binding loop sequence. In some examples, the modified MS2-binding loop sequences includes about 6 nucleotide changes, thereby increasing GC content of the MS2-binding loop sequence.

In some examples, the nucleotide changes to the native MS2-binding loop sequence shortens repetitive content, such as decreasing repetitive content by about 5%, about 8%, about 10%, about 15%, about 20%, about 30%, about 40%, or about 50%, or more. In some examples, the decrease is about 1 to 5%, about 1 to 8%, about 1 to 10%, about I to 15%, about 5 to 10%, about 5 to 20%, about 5 to 30%, about 5 to 40%, about 5 to 50%, about 5 to 60%, about 5 to 75%, about 10 to 20%, about 10 to 30%, about 10 to 40%, about 10 to 50%, about 10 to 60%, about 10 to 75%, about 20 to 30%, about 20 to 40%, about 20 to 50%, about 20 to 60%, about 20 to 75%, about 30 to 40%, about 30 to 50%, about 30 to 60%, about 30 to 75%, about 40 to 50%, about 40 to 60%, about 40 to 75%, about 50 to 60%, or about 50 to 75%. In some examples, the modified MS2-binding loop sequences includes about 2 nucleotide changes, thereby decreasing repetitive content of the MS2-binding loop sequence. In some examples, the modified MS2-binding loop sequences includes about 6 nucleotide changes, thereby decreasing repetitive content of the MS2-binding loop sequence. In further examples, the repetitive content is shortened or decreased by deleting one or more repetitive nucleotides.

In specific examples, the modified MS2-binding loop sequence includes at least 70%, at least 80%, at least 85%, at least 90%, at least 95%, at least 98%, at least 99%, or 100% sequence identity to one or more of SEQ ID NO: 17, 18, or 19. In a non-limiting example, the modified MS2-binding loop sequence includes at least 95% sequence identity to one or more of SEQ ID NO: 17, 18, or 19. In further examples, the modified MS2-binding loop sequence includes or consists of the sequence tgctgaacatgaggatcacccatgtctgcagcagca (SEQ ID NO: 17), gggccaacatgaggatcacccatgtctgcagggccc (SEQ ID NO: 18), or ggccagcatgaggatcacccatgcctgcagggcc (SEQ ID NO: 19).

### Exemplary Promoters

The promoter (e.g., the first or second promoter of the multiplex crRNA or multiplex sgRNA, for example 110, 111, 112, 113) can be any suitable promoter. For example, a pol III promoter (e.g., a U6 or Hl promoter); a pol II promoter (e.g., the retroviral Rous sarcoma virus (RSV) LTR promoter, optionally with the RSV enhancer); a cytomegalovirus (CMV) promoter, optionally with the CMV enhancer; a SV40 promoter; a dihydrofolate reductase promoter; a β-actin promoter; a phosphoglycerol kinase (PGK) promoter; Spc5.12 (muscle specific); CW3SL; and/or a EF1a promoter. In some examples, the promoter is specific for a certain cell type or organ (e.g. Spc5.12). In other examples, the promoter is ubiquitous (e.g., EF1a). In some examples, the promoter is a minimal promoter, such as cytomegalovirus (CMV), human b-actin (hACTB), human elongation factor-la (hEF-1a), and/or cytomegalovirus early enhancer/chicken b-actin (CAG) promoters (e.g., the promoters described in Papadakis et al., Current Gene Therapy, 4:89-113, 2004; Damdindorj et al., PLOS ONE 9(8):e106472, 2014). In one example, one or more of the promoters 110, 111, 112, 113 is a liver-specific promoter, such as albumin promoter, hepatitis B virus core protein promoter, hemopexin promoter, or human alpha 1-antitrypsin promoter.

In some examples the first promoter 110, 112 and the second promoter 111, 113 consist of or include different sequences. In other examples, the first promoter 110, 112 and the second promoter 111, 113 consist of or include the same sequence. In some examples, the first promoter 110, 112 and/or the second promoter 111, 113 is a mU6, hU6, H1, or 7SK promoter. In specific, non-limiting examples, the first promoter 110, 112 is hU6 or mU6, and the second promoter 111, 113 is hU6 or mU6. In some examples, the promoter 110-113 confers tropism for a specific tissue or cell-type, for example, Spc5.12 (muscle specific) or Col1a2 (fibroblast specific), or is inducible in response to stimuli. It will be appreciated by those skilled in the art that the promoter selection can depend on factors such as the choice of tissue or cell target, host cell to be transformed, level of expression desired, etc.

### Exemplary Cleavage Sites

A cleavage site, for example, the first 120 or second 121 cleavage site of the multiplex crRNA or the first 122 or second cleavage 123 site of the multiplex sgRNA, is a sequence that when transcribed into RNA is capable of being cleaved. Suitable cleavage mechanisms include self-cleavage, such as a self-cleaving ribozyme, or cleavage through an endogenous mechanism of a host cell, such as pre-t-RNA cleavage.

In some examples, the cleavage site (e.g., 120, 121, 122, 123) is a self-cleaving RNA. In some examples, the cleavage site (e.g., 120, 121, 122, 123) includes or consists of a pre-tRNA sequence. In other examples, the cleavage site (e.g., 120, 121, 122, 123) includes or consists of a self-cleaving ribozyme, such as a hepatitis delta virus hammerhead ribozyme (HDV-HH). The first cleavage site 120, 122 and the second cleavage site 121, 123 can consist of or include different sequences, or may consist of or include the same sequence. In specific, non-limiting examples, the first cleavage site 120, 122 is a pre-tRNA sequence and the second cleavage site 121, 123 is a self-cleaving ribozyme, such as a hammerhead. In other, non-limiting examples, the first cleavage site 120, 122 is a pre-tRNA sequence and the second cleavage site 121, 123 is also a pre-tRNA sequence. In some examples, the first cleavage site 120, 122 is a pre-tRNA sequence, and the second cleavage site 121, 123 is a pre-tRNA sequence from a different organism. In a non-limiting example, one cleavage site can be a pre-tRNA from yeast and the other can be a pre-tRNA from a plant, such as Zea mays. In specific, non-limiting examples, the first cleavage site 120 of the multiplex crRNA 100 includes or consists of SEQ ID NO: 20 or SEQ ID NO: 21 and the second cleavage site 121 includes or consists of SEQ ID NO: 22. In other specific, non-limiting examples, the first cleavage site 122 of the multiplex sgRNA 200 includes or consists of SEQ ID NO: 20 or SEQ ID NO: 21 and the second cleavage site 123 of the multiplex sgRNA 200 includes or consists of SEQ ID NO: 20 or SEQ ID NO: 21.

### A. VECTORS THAT INCLUDE MULTIPLEX crRNAs AND MULTIPLEx sgRNAs

Also provided are vectors, such as a viral vector (e.g., retrovirus, lentivirus, adenovirus, adeno-associated virus, or herpes simplex virus) or plasmid, which includes one or more nucleic acid molecules encoding multiplex crRNA, multiplex sgRNA, or both. In some examples, the vector is an AAV vector, such as an AAV1 vector, AAV2 vector, AAV3 vector, AAV4 vector, AAV5 vector, AAV6 vector, AAV7 vector, AAV8 vector, AAV9 vector, AAV10 vector, AAV11 vector, AAV12 vector AAV-PHP.B vector, AAV-PHP.eB vector, or AAV-PHP.S vector. In a specific, non-limiting example, the vector is an AAV9 vector. In some examples, the vector is an adenovirus vector, such as Ad5. The vectors can include other elements, such as a gene encoding a selectable marker, such as an antibiotic, such as puromycin, hygromycin, or a detectable marker such as a fluorophore (e.g. GFP or RFP) or a luciferase protein. The vector can include naturally occurring or non-naturally occurring nucleotides or ribonucleotides. The disclosed vectors can be used in the methods, compositions, and kits provided herein.

### B. COMPOSITIONS AND KITS THAT INCLUDE MULTIPLEX crRNAs AND MULTIPLEX sgRNAs

Also provided are compositions and kits that include one or more nucleic acids encoding the multiplex crRNAs or multiplex sgRNAs provided herein, or one or more multiplex crRNAs or multiplex sgRNAs provided herein. For example, the composition can include one or more nucleic acids encoding the disclosed multiplex crRNAs or multiplex sgRNAs, the disclosed RNA molecules encoded by the multiplex crRNAs or multiplex sgRNAs, the disclosed vectors encoding the multiplex crRNAs or multiplex sgRNAs, or a ribonucleoprotein (RNP) complex including the multiplex crRNAs or multiplex sgRNAs, and a pharmaceutically acceptable carrier (e.g., saline, water, or PBS). In some examples, one or more nucleic acids encoding the multiplex crRNAs or multiplex sgRNAs, or the RNAs thereof, are present in a cell that is part of the composition. In some examples, the composition is a liquid, a lyophilized powder, or cryopreserved.

The compositions are suitable for formulation and administration in vitro or in vivo. Suitable carriers and their formulations are described in Remington: The Science and Practice of Pharmacy, 22^{nd} Edition, Loyd V. Allen et al., editors, Pharmaceutical Press (2012). Pharmaceutically acceptable carriers include materials that are not biologically or otherwise undesirable, i.e., the material is administered to a subject without causing undesirable biological effects or interacting in a deleterious manner with the other components of the pharmaceutical composition in which it is contained. If administered to a subject, the carrier is optionally selected to minimize degradation of the active ingredient (e.g., a vector comprising the multiplex crRNAs and/or multiplex sgRNAs) and to minimize adverse side effects in the subject.

In some embodiments, the disclosed compositions for administration are dissolved in a pharmaceutically acceptable carrier, such as an aqueous carrier. A variety of aqueous carriers can be used, e.g., buffered saline and the like. These solutions can be sterile and generally free of undesirable matter. These compositions may be sterilized. The compositions may contain pharmaceutically acceptable auxiliary substances as required to approximate physiological conditions, such as pH adjusting and buffering agents, toxicity adjusting agents, and the like, for example, sodium acetate, sodium chloride, potassium chloride, calcium chloride, sodium lactate, and the like. The concentration of active agent in these formulations can vary and can be selected primarily based on fluid volumes, viscosities, body weight, and the like in accordance with the particular mode of administration selected and the subject's needs.

Pharmaceutical formulations can be prepared by mixing the disclosed nucleic acid molecules, RNA molecules, vectors, or RNP complexes, having the desired degree of purity with optional pharmaceutically acceptable carriers, excipients, or stabilizers. Such formulations can be lyophilized formulations or aqueous solutions.

Acceptable carriers, excipients, or stabilizers are nontoxic to recipients at the dosages and concentrations used. Acceptable carriers, excipients, or stabilizers can be acetate, phosphate, citrate, and other organic acids; antioxidants (e.g., ascorbic acid) preservatives, and low molecular weight polypeptides; proteins, such as serum albumin or gelatin, or hydrophilic polymers, such as polyvinylpyllolidone; and amino acids, monosaccharides, disaccharides, and other carbohydrates including glucose, mannose, or dextrins; chelating agents; ionic and non-ionic surfactants (e.g., polysorbate); salt-forming counter-ions, such as sodium; metal complexes (e.g. Zn-protein complexes); and/or non-ionic surfactants.

Formulations suitable for oral administration can include (a) liquid solutions, such as an effective amount of the disclosed nucleic acid molecules, RNA, or vectors, RNP complexes, or combinations thereof, suspended in diluents, such as water, saline, or PEG 400; (b) capsules, sachets or tablets, each containing a predetermined amount of the active ingredient, as liquids, solids, granules, or gelatin; (c) suspensions in an appropriate liquid; and (d) suitable emulsions.

Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can include the active ingredient in a flavor, e.g., sucrose, as well as pastilles including the active ingredient in an inert base, such as gelatin and glycerin or sucrose and acacia emulsions, gels, and the like containing, in addition to the active ingredient, carriers.

The disclosed nucleic acid molecules (e.g., DNA, such as cDNA), RNA molecules, vectors, or RNP complexes, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the provided methods, compositions can be administered, for example, by intravenous infusion, orally, topically, intraperitoneally, intravesically, intratumorally, or intrathecally. Parenteral administration, intratumoral administration, and intravenous administration are the preferred methods of administration. The formulations of compounds can be presented in unit-dose or multi-dose sealed containers, such as ampules and vials.

Injection solutions and suspensions can be prepared from sterile powders, granules, and tablets of the kind previously described. Cells transduced or infected with the disclosed nucleic acids for ex vivo therapy can also be administered intravenously or parenterally as described above.

The pharmaceutical preparation can be in unit dosage form. In such form, the preparation is subdivided into unit doses containing appropriate quantities of the active component. Thus, the pharmaceutical compositions can be administered in a variety of unit dosage forms depending upon the method of administration. For example, unit dosage forms suitable for oral administration include, but are not limited to, powder, tablets, pills, capsules, and lozenges.

Also provided are kits that include one or more nucleic acids encoding the disclosed multiplex crRNAs or multiplex sgRNAs (which may be part of a vector, such as an AAV vector, and/or may be present in a cell, such as a mammalian cell) or one or more multiplex crRNAs or multiplex sgRNAs provided herein. The kits can further include a nucleic acid encoding a Cas9 protein or dCas9 protein (which may be part of a vector, such as an AAV vector, and/or may be present in a cell, such as a mammalian cell). In some examples, the kits further include a Cas9 protein or dCas9 protein. The kits can further include a nucleic acid encoding an MS2-transcriptional activator fusion protein (e.g., MS2-p65-HSF1), which may be part of a vector (e.g., AAV vector) and/or may be present in a cell, such as a mammalian cell. In some examples, the nucleic acid encoding a Cas9 protein or dCas9 protein and the nucleic acid encoding an MS2-transcriptional activator fusion protein are part of a single viral vector (e.g., AAV vector). In some examples, the nucleic acid encoding an MS2-transcriptional activator fusion protein encodes MS2-p65-HSF1, such as a sequence encoding a protein sequence having at least 90%, at least 95%, at least 98%, at least 99%, or 100% sequence identity to SEQ ID NO: 35.

In one example, the composition or kit includes a ribonucleoprotein (RNP) complex (e.g., a mTGA complex) composed of one or more Cas9 or dCas9 proteins and one or more of the disclosed crRNA and modified tracrRNA, or modified sgRNAs, and one or more transcriptional activators (e.g., MS2-p65-HSF1). In some examples, the RNP complex includes the disclosed crRNA and the modified tracrRNA. In further examples, the RNP complex includes the disclosed modified sgRNA (including the disclosed dgRNAs).

In further examples, the composition or kit includes a vector encoding a Cas9 or dCas9 protein and a vector encoding one or more disclosed crRNAs or modified sgRNA (including the dgRNAs) and encoding an MS2-transcriptional activator fusion protein. In one example, the composition or kit includes a cell, such as a bacterial cell or eukaryotic cell, that includes a Cas9 or dCas9 protein, a Cas9 or dCas9 protein coding sequence, a crRNA or modified sgRNA molecule, a nucleic acid encoding an MS2-transcriptional activator fusion protein, MS2-transcriptional activator fusion protein (e.g., MS2-p65-HSF1), or combinations thereof. In one example, the composition or kit includes a cell-free system that includes: a Cas9 or dCas9 protein, a Cas9 or dCas9 protein coding sequence, a disclosed RNA molecule (e.g., crRNA, modified tracrRNA, modified sgRNA, multiplex crRNA, multiplex sgRNA), a nucleic acid encoding a multiplex crRNA or multiplex sgRNA, MS2-transcriptional activator fusion protein (e.g., MS2-p65-HSF1), a nucleic acid encoding an MS2-transcriptional activator fusion protein, or combinations thereof.

In some examples, the kit includes a delivery system (e.g., liposome, a particle, an exosome, a microvesicle, a viral vector, or a plasmid), and/or a label (e.g., a peptide or antibody that can be conjugated either directly to an RNP or to a particle containing the RNP to direct cell type specific uptake/enhance endosomal escape/enable blood-brain barrier crossing etc.). In some examples, the kits further include cell culture or growth media, such as media appropriate for growing bacterial, plant, insect, or mammalian cells. In some examples, components of the kit are in separate containers (such as glass or plastic vials).

### C. CELLS THAT INCLUDE MULTIPLEX crRNAs AND MULTIPLEX sgRNAs

Cells are provided that include one or more nucleic acids encoding the multiplex crRNAs or multiplex sgRNAs provided herein, or one or more multiplex crRNAs or multiplex sgRNAs provided herein. In some examples, such cells also include a Cas9 or dCas9 protein. In some examples, such cells also include an MS2-transcriptional activator fusion protein. Nucleic acid molecules encoding multiplex crRNAs and multiplex sgRNAs (including RNA molecules thereof), as well as nucleic acid molecules encoding a Cas9, a dCas9, and/or an MS2-transcriptional activator fusion protein, can be introduced into cells to generate transformed (e.g., recombinant) cells. Such recombinant cells can be used in the methods, compositions, and kits provided herein. In some examples, such cells are generated by introducing Cas9, dCas9, and/or MS2-transcriptional activator fusion protein and one or more multiplex crRNA and multiplex sgRNA RNA molecules into the cell, for example, as a ribonucleoprotein (RNP) complex.

Such recombinant cells can be eukaryotic or prokaryotic. Examples of such cells include, but are not limited to, bacteria, archaea, plant, fungal, yeast, insect, and mammalian cells, such as Lactobacillus, Lactococcus, Bacillus (such as B. subtilis), Escherichia (such as E. coli), Clostridium, Saccharomyces or Pichia (such as S. cerevisiae or P. pastoris), Kluyveromyces lactis, Salmonella typhimurium, Drosophila cells, C. elegans cells, Xenopus cells, SF9 cells, C129 cells, 293 cells, Neurospora, and immortalized mammalian cell lines (e.g., Hela cells, myeloid cell lines, liver cell lines, and lymphoid cell lines). In one example, the cell is a prokaryotic cell, such as a bacterial cell, such as E. coli.

In one example, the cell is a eukaryotic cell, such as a mammalian cell, such as a human cell. In one example, the cell is primary eukaryotic cell, a stem cell, a tumor/cancer cell, a circulating tumor cell (CTC), a blood cell (e.g., T cell, B cell, NK cell, Tregs, etc.), hematopoietic stem cell, specialized immune cell (e.g., tumor-infiltrating lymphocyte or tumor-suppressed lymphocytes), a stromal cell in the tumor microenvironment (e.g., cancer-associated fibroblasts, etc.), pancreatic cell, kidney cell, liver cell, or muscle cell. In one example, the cell is a brain cell (e.g., neurons, astrocytes, microglia, retinal ganglion cells, rods/cones, etc.) of the central or peripheral nervous system).

In one example, a cell is part of (or obtained from) a biological sample, such as a biological specimen containing genomic DNA, RNA (e.g., mRNA), protein, or combinations thereof obtained from a subject. Examples include, but are not limited to, peripheral blood, serum, plasma, urine, saliva, sputum, tissue biopsy, fine needle aspirate, surgical specimen, and autopsy material.

In one example, the cell is from a tumor, such as a hematological tumor (e.g., leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (including low-, intermediate-, and high-grade), multiple myeloma, Waldenström's macroglobulinemia, heavy chain disease, myelodysplastic syndrome, mantle cell lymphoma, and myelodysplasia) or solid tumor (e.g., sarcomas and carcinomas: fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, and bladder carcinoma as well as CNS tumors (such as a glioma, astrocytoma, medulloblastoma, craniopharyogioma, ependymoma, pinealoma, hemangioblastoma, acoustic neuroma, oligodendroglioma, menangioma, melanoma, neuroblastoma and retinoblastoma)).

### IV. MULTIPLEX TARGETED GENE ACTIVATION (mTGA) SYSTEM

Also provided is a multiplex targeted gene activation (mTGA) system. The system can include a first vector (such as a viral vector, e.g., AAV, or lentiviral vector) that includes a nucleic acid encoding a Cas9 or dCas9 (whose expression can be driven by a promoter) and a second vector (such as a viral vector, e.g., AAV, or lentiviral vector) that includes one or more nucleic acids encoding one or more of a multiplex crRNA or multiplex sgRNA disclosed herein, and a nucleic acid encoding an MS2-transcriptional activator fusion protein (such as MS2-p65-HSF1, whose expression can be driven by a promoter). In some examples, the nucleic acid encoding a MS2-transcriptional activator fusion protein encodes MS2-p65-HSF1, such as a sequence encoding a protein sequence having at least 90%, at least 95%, at least 98%, at least 99%, or 100% sequence identity to SEQ ID NO: 35.

In some examples, the first and second vector are viral vectors, such as an adeno-associated viral (AAV) vectors (e.g., an AAV1 vector, AAV2 vector, AAV3 vector, AAV4 vector, AAV5 vector, AAV6 vector, AAV7 vector, AAV8 vector, AAV9 vector, AAV10 vector, AAV11 vector, AAV12 vector, AAV-PHP.B vector, AAV-PHP.eB vector, or AAV-PHP.S vector) or an adenoviral vector (e.g., Ad5). In one example, the first and second vector are AAV9 or Ad5 vectors. In some examples, the first and first and second vector are AAV8 vectors. In some examples, the AAV vector used has tropism for a specific tissue or cell-type, such as a kidney cell, muscle cell, or pancreatic cell.

In some examples, the first vector includes a nucleic acid encoding a Cas9 protein, such as a Streptococcus pyogenes Cas9 protein. In some examples, the first vector includes a nucleic acid encoding a Cas9 protein, such as a nucleic acid molecule encoding a protein having at least 90%, at least 95%, at least 98%, at least 99%, or 100% sequence identity to SEQ ID NO: 31, wherein the Cas9 protein has endonuclease activity. In some examples, the first vector includes a nucleic acid encoding a dCas9 protein, such as a dCas9 protein with reduced or no endonuclease activity. In some examples, the first vector includes a nucleic acid encoding a dCas9 protein, such as a nucleic acid molecule encoding a protein having at least 90%, at least 95%, at least 98%, at least 99%, or 100% sequence identity to SEQ ID NO: 33, wherein the dCas9 protein has reduced or endonuclease activity. In some examples, the dCas9 protein encoded by the nucleic acid molecule has a D10A, E762A, D839A, H840A, N854A, N863A, D986A, or combinations thereof, mutation.

In some examples, the first vector includes a nucleic acid encoding a Cas9 or dCas9 protein and does not encode a transcriptional activator, such as VP64, P65, MyoDI, HSF1, RTA, SET7/9, or any combination thereof. Thus, in some examples, the Cas9 or dCas9 protein encoded by the first vector is not a Cas9-transcriptional activator fusion protein or a dCas9-transcriptional activator fusion protein.

The second vector includes one or more nucleic acids encoding a multiplex crRNA or multiplex sgRNA disclosed herein, such as one having at least 90%, at least 95%, at least 98%, at least 99%, or 100% sequence identity to SEQ ID NO: 1, 2, 3, 4, 5, 6, 53, 54, or 55. In one example, the encoded multiplexed crRNA or modified sgRNA has at least 95% sequence identity to SEQ ID NO: 1, 2, 3, 4, 5, 6, 53, 54, or 55.

The second vector also includes a nucleic acid encoding an MS2-transcriptional activator fusion protein. MS2-transcriptional activator fusion proteins include an MS2 domain fused directly or indirectly (e.g., via a linker) with a transcriptional activation domain. Exemplary transcriptional activation domains include VP64, p65, MyoDI, HSF1, RTA, SET7/9, or any combination thereof. In some examples, the nucleic acid encoding an MS2-transcriptional activator fusion protein encodes MS2-p65-HSF1, such as a sequence encoding a protein sequence having at least 90%, at least 95%, at least 98%, at least 99%, or 100% sequence identity to SEQ ID NO: 35.

In some examples, the mTGA system allows for multiple genes to be targeted. In some examples, the mTGA system further includes one or more additional multiplex crRNAs, multiplex sgRNAs, crRNA, modified sgRNAs (including dgRNAs). Additional multiplex crRNAs, multiplex sgRNAs, crRNA, or modified sgRNAs, can be used, for example, to target different genes of interest. Such additional multiplex crRNAs, multiplex sgRNAs, crRNA, or modified sgRNAs, can be on additional vectors, or can also be on the second vector.

### V. METHODS OF TARGETED GENE ACTIVATION

Provided herein are methods of increasing expression (e.g., activating expression) of at least one gene product in vitro or in a subject. The gene product whose expression is increased can be the gene itself (e.g., DNA), an RNA (such as mRNA, miRNA, and non-coding RNA), or gene product (e.g., protein). When used in vitro, expression can be increased in a cell, such as a eukaryotic or prokaryotic cell, for example, a mammalian cell. When used in vivo, expression can be increased in a subject, such as a mammal (e.g., mouse, non-human primate, or other veterinary subject) or a human.

Methods of using the disclosed multiplex crRNAs, multiplex sgRNAs, and mTGA system are also provided herein. Such methods can be used to increase expression of at least one target gene product in a subject, such as a gene whose expression is decreased in the subject. In some examples, the disclosed methods treat a disease in the subject caused by decreased expression of a gene (a causative gene). In some examples, the target gene is the causative gene. In other examples, the target gene is not the causative gene, and instead increased expression of the target gene compensates for loss of function of the causative gene, for example, when the target gene is a functional analog of the causative gene. In some examples, the methods increases expression of the target gene or gene product by at least about 10%, about 20%, about 30%, about 40%, about 50%, about 60%, about 70%, about 80%, about 90%, about 95%, about 100%, about 200%, about 300%, about 400%, or about 500%. In further examples, the methods increases expression of the target gene or gene product by about 10 to 500%, about 10 to 400%, about 10 to 300%, about 10 to 200%, about 10 to 100%, about 10 to 90%, about 10 to 80%, about 10 to 70%, about 10 to 60%, about 10 to 50%, about 10 to 40%, about 10 to 30%, about 10 to 20%, about 20 to 500%, about 30 to 500%, about 40 to 500%, about 50 to 500%, about 60 to 500%, about 70 to 500%, about 80 to 500%, about 90 to 500%, about 100 to 500%, about 200 to 500%, about 300 to 500%, about 400 to 500%, about 25 to 100%, about 25 to 200%, about 50 to 100%, about 50 to 200%, about 50 to 300%, about 50 to 400%, about 50 to 500%, about 100 to 200%, about 100 to 300%, about 100 to 400%, about 100 to 500%, about 200 to 300%, about 200 to 400%, or about 200 to 500%.

In some examples, the method is an in vivo method of increasing expression (e.g., activating expression) of at least one gene product in a subject. In some examples, the gene product is a product of the target gene. The method includes administering a therapeutically effective amount of a multiplex targeted gene activation (mTGA) system to a subject. The components of the mTGA system infect a cell (e.g., a cell in the subject, such as a cell of the muscle, liver, heart, lung, kidney, spinal cord, or stomach, such as a liver or muscle cell), thereby increasing expression of the at least one gene product in the subject.

In some examples, the method is an in vitro method of increasing expression (e.g., activating expression) of at least one gene product in a cell or cell-free system. In some examples, the gene product is a product of the target gene. The method includes contacting an effective amount of a multiplex targeted gene activation (mTGA) system with the cell or cell-free system. The components of the mTGA system infect an in vitro cell (e.g., mammalian cell), or are expressed in the cell-free system, thereby increasing expression of the at least one gene product in the infected cell or cell-free system.

The mTGA system is administered in accord with known methods, such as systemic or local administration. In specific examples, intravenous administration, e.g., as a bolus or by continuous infusion over a period of time, or intramuscular, intraperitoneal, intracerobrospinal, subcutaneous, intra-articular, intrasynovial, intrathecal, oral, topical, intratumoral, or inhalation routes are used. In one example administration is directly to the liver or hepatic vein or hepatic artery. Thus, the disclosed mTGA system can be administered via any of several routes of administration, including topically, orally, parenterally, intravenously, intra-articularly, intraperitoneally, intramuscularly, subcutaneously, intracavity, transdermally, intrahepatically, intracranially, intratumorally, intraosseously, nebulization/inhalation, into the liver or vasculature thereof, or by installation via bronchoscopy. Thus, the compositions are administered in a number of ways depending on whether local or systemic treatment is desired, and the area to be treated.

An effective amount of the mTGA system disclosed herein can be based, at least in part, on the particular vector used; the individual's size, age, gender; and the size and other characteristics of the proliferating cells. For example, for treatment of a human, at least 10³ viral genomes (vg) per kg of body weight of a viral vector is used, such as at least 10⁴, at least 10⁵, at least 10⁶, at least 10⁷, at least 10⁸, at least 10⁹, at least 10¹⁰, at least 10¹¹, at least 10¹², at least 10¹³, at least 10¹⁴, at least 10¹⁵, at least 10¹⁶, at least 10¹⁷, at least 10¹⁸, at least 10¹⁹, or at least 10²⁰ vg/kg of body weight, for example, approximately 10³ to 10²⁰, 10⁹ to 10¹⁶, 10¹² to 10¹⁵, or 10¹³ to 10¹⁴ vg/kg of body weight of a viral vector is used.

The disclosed compositions, such as a viral vector (e.g., AAV vector), can be administered in a single dose or in multiple doses (e.g., two, three, four, six, or more doses). Multiple doses can be administered concurrently or consecutively (e.g., over a period of days or weeks).

The mTGA system used in the method can include (1) a first vector including a nucleic acid encoding a Cas9 protein or dCas9 protein and (2) a second vector including a multiplexed crRNA or multiplexed sgRNA disclosed herein and a nucleic acid encoding an MS2-transcriptional activator fusion protein. In some examples, the first and second vector are adeno-associated viral (AAV) vectors, such as an AAVI vector, AAV2 vector, AAV3 vector, AAV4 vector, AAV5 vector, AAV6 vector, AAV7 vector, AAV8 vector, AAV9 vector, AAV10 vector, AAV11 vector, AAV12 vector AAV-PHP.B vector, AAV-PHP.eB vector, or AAV-PHP.S vector. In one example, the first and second vector are AAV9 vectors. In some examples, the AAV vector used has tropism for a specific tissue or cell-type, such as a kidney cell, skeletal muscle cell, liver cell, or pancreatic cell (examples provided elsewhere herein).

When selecting elements for the disclosed mTGA system, which allow for gene activation without introducing DNA double strand breaks, either the Cas9 protein used or the modified sgRNA need to be a dead form, or both. Thus, in some examples, a dCas9 protein (e.g., SEQ ID NO: 33) is used with the multiplex crRNA or multiplex sgRNA. In some examples, a Cas9 protein (e.g., SEQ ID NO: 31) is used with multiplex crRNA or multiplex sgRNA, wherein the modified sgRNAs are dgRNAs.

In some examples, the first vector includes a nucleic acid encoding a Cas9 protein, such as a Streptococcus pyogenes Cas9 protein. In some examples, the first vector includes a nucleic acid encoding a Cas9 protein, such as a nucleic acid molecule encoding a protein having at least 90%, at least 95%, at least 98%, at least 99%, or 100% sequence identity to SEQ ID NO: 31, wherein the Cas9 protein has endonuclease activity. In some examples, the first vector includes a nucleic acid encoding a dCas9 protein, such as a dCas9 protein with reduced or no endonuclease activity. In some examples, the first vector includes a nucleic acid encoding a dCas9 protein, such as a nucleic acid molecule encoding a protein having at least 90%, at least 95%, at least 98%, at least 99%, or 100% sequence identity to SEQ ID NO: 33, wherein the dCas9 protein has reduced or endonuclease activity. In some examples, the dCas9 protein encoded by the nucleic acid molecule has a D10A, E762A, D839A, H840A, N854A, N863A, D986A, or combinations thereof, mutation.

In some examples, the first vector includes a nucleic acid encoding a Cas9 or dCas9 protein does not encode a transcriptional activator, such as VP64, P65, MyoDI, HSF1, RTA, SET7/9, or any combination thereof. Thus, in some examples, the Cas9 or dCas9 protein encoded by the first vector is not a Cas9-transcriptional activator fusion protein or a dCas9-transcriptional activator fusion protein.

In some embodiments, the second vector encodes a multiplex crRNA or multiplex sgRNA disclosed herein, such as one having at least 90%, at least 95%, at least 98%, at least 99%, or 100% sequence identity to SEQ ID NO: 1, 2, 3, 4, 5, 6, 53, 54, or 55. In a non-limiting example, the encoded multiplexed crRNA has at least 95% sequence identity to SEQ ID NO: 1 or 2. In another non-limiting example, the encoded multiplexed sgRNA has at least 95% sequence identity to SEQ ID NO: 3, 4, 5, 6, 53, 54, or 55.

The second vector also includes a nucleic acid encoding an MS2-transcriptional activator fusion protein. MS2-transcriptional activator fusion proteins include an MS2 domain fused directly or indirectly (e.g., via a linker) with a transcriptional activation domain. Exemplary transcriptional activation domains include VP64, p65, MyoDI, HSF1, RTA, SET7/9, or any combination thereof. In some examples, the nucleic acid encoding an MS2-transcriptional activator fusion protein encodes MS2-p65-HSF1, such as a sequence encoding a protein sequence having at least 90%, at least 95%, at least 98%, at least 99%, or 100% sequence identity to SEQ ID NO: 35.

In some examples, the mTGA system further includes one or more additional multiplex crRNAs, multiplex sgRNAs, crRNA, or modified sgRNAs (including dgRNAs), or nucleic acid molecule encoding such. Additional multiplex crRNAs, multiplex sgRNAs, crRNA, or sgRNAs, or nucleic acid molecules encoding such, can be used, for example, to target different genes of interest. Such additional multiplex crRNAs, multiplex sgRNAs, crRNA, or modified sgRNAs can be on additional vectors, or can also be on the second vector.

In one example, the Cas9, dCas9, and/or MS2-transcriptional activator fusion protein is expressed in a recombinant cell, such as E. coli, and purified. The resulting purified Cas9, dCas9, and/or MS2-transcriptional activator fusion protein, along with one or more of the disclosed encoded multiplex crRNA, multiplex sgRNA, or RNA products thereof, is then introduced into a cell or organism where one or more genes can be upregulated. In some examples, the Cas9, dCas9, and/or MS2-transcriptional activator fusion protein and encoded multiplex crRNA, multiplex sgRNA, or RNA products thereof, are introduced as separate components into the cell/organism. In other examples, the purified Cas9, dCas9, and/or MS2-transcriptional activator fusion is complexed with the disclosed RNA molecule (e.g., RNA molecule of the disclosed multiplex crRNA or multiplex sgRNA), and this ribonucleoprotein (RNP) complex is introduced into target cells (e.g., using transfection or injection). In some examples, the Cas9, dCas9, and/or MS2-transcriptional activator fusion protein and RNA molecule (or nucleic acid molecule encoding such) are injected into an embryo (such as a human, mouse, zebrafish, or Xenopus embryo). Once the Cas9 or dCas9 protein, MS2-transcriptional activator fusion protein, and RNA molecule (or nucleic acid molecule encoding such) are in the cell, expression of one or more target nucleic acid molecules can be activated. One or more nucleic acid molecules or genes can be targeted by the disclosed methods, such as about 1, about 2, about 3, about 4, or about 5, about 6, about 7, about 8, about 9, or about 10 different nucleic acid molecules or genes in a cell or organism. In some examples, about 1 to 10, about 1 to 9, about 1 to 8, about 1 to 7, about 1 to 6, about 1 to 5, about 1 to 4, about 1 to 3, about 1 to 2, about 2 to 10, about 3 to 10, about 4 to 10, about 5 to 10, about 6 to 10, about 7 to 10, about 8 to 10, about 9 to 10, about 2 to 4, about 2 to 6, about 2 to 8, about 2 to 10, about 4 to 6, about 4 to 8, about 4 to 10, about 6 to 8, about 6 to 10, or about 8 to 10, different nucleic acid molecules or genes are targeted by the disclosed methods. In some examples, the disclosed methods are used to treat or prevent a disease associated with no or reduced expression of one or more genes (e.g., a reduction of at least 10%, at least 20%, at least 30%, at least 40%, at least 50%, at least 60%, at least 70%, at least 80%, at least 90%, at least 95%, at least 99%, or 100% reduction). In one example, the target is associated with a disease, such as type I diabetes, Duchenne muscular dystrophy, or acute kidney disease. In some examples, the disease is of the liver, muscle, pancreas, or kidney. In some examples, the disease is a disease of the liver, such as Alagille Syndrome; alpha-1 antitrypsin deficiency (alpha-1); biliary atresia; cirrhosis; galactosemia; Gilbert syndrome; hemochromatosis; Lysosomal acid lipase deficiency (LAL-D); non-alcoholic fatty liver disease (NAFLD); primary biliary cholangitis (PBC); primary sclerosing cholangitis (PSC); type I glycogen storage disease (GSD I); and Wilson disease. In some examples, the gene or gene product targeted (e.g., is activated) is one or more of Fst, Pdx1, klotho, utrophin, interleukin 10, insulin 1, insulin 2, Pcskl, Six2, Foxa3, Gata4, HNF1a, and HNF4a. In a specific, non-limiting example, the disease is muscular dystrophy and the causative gene is dystrophin and the target gene is utrophin. In another non-limiting example, the disease is a liver disease, such as liver fibrosis and/or cirrhosis, and the target gene is Foxa3, Gata4, HNF1a, and/or HNF4a.

Specific examples of diseases that can be treated, along with genes that can be targeted (e.g., activated) with the disclosed methods, are provided in Table 1 and Table 2. In certain embodiments, the targeting sequence is complementary to a sequence at least within about 10 nt, about 25 nt, about 50 nt, about 60 nt, about 70 nt, about 80 nt, about 90 nt, about 100 nt, about 110 nt, about 120 nt, about 130 nt, about 140 nt, about 150 nt, about 175 nt, about 200 nt, about 300 nt, about 400 nt, or about 500 nt of a transcriptional start site of a target gene.

### VI. REPORTERS

Disclosed herein are systems, kits, and methods for measuring gene activation, such as where Cas9 (e.g., Cas9 or dCas9) is expressed or with a Cas9 expression step. The systems, kits, and methods for measuring gene activation herein can be used, for example, to assay the efficiency of gene activation (e.g., the efficiency of gene activation by the mTGA system disclosed herein) and/or isolating or sorting cells (e.g., isolating or sorting cells with gene activation, or isolating or sorting cells without gene activation).

Provided herein are systems and kits for measuring gene activation when Cas9 is expressed. In some examples, the systems and kits include at least one gene activation vector and at least one reporter vector. Cas9, including Cas9 or dCas9, can be expressed constitutively or inducibly as well as endogenously or exogenously using any suitable method, kit, system, or composition, including the methods, kits, systems, and compositions disclosed herein, such as using a vector (e.g., a viral vector, such as an AAV vector) that encodes Cas9 (e.g., Cas9 or dCas9). In some examples, the at least one gene activation vector includes a multiplex crRNA or multiplex sgRNA and at least one transcriptional activator protein. In some examples, the at least one reporter vector includes a target sequence of the multiplex crRNA or multiplex sgRNA and at least one reporter protein, in which the reporter protein is positioned downstream of the target sequence.

In some examples, the methods include injecting a subject with at least one gene activation vector and at least one reporter vector. Any suitable injection method can be used, including subcutaneous, intramuscular, intravenous, intraperitoneal, intracardiac, intraarticular, injection into the liver or vasculature thereof, and/or intracavernous injection of any amount of the at least one gene activation vector and at least one reporter vector (e.g., an effective amount of a vector, such as that described herein).

The vector of the at least one gene activation vector or the at least one reporter vector can be any suitable vector, such as any vector described herein. In some examples, the vector is a viral vector or plasmid (e.g., retrovirus, lentivirus, adenovirus, adeno-associated virus, or herpes simplex virus). In specific examples, the vector is an AAV vector (e.g., an AAV9 vector). In some examples, the AAV vector has tropism for a specific tissue or cell-type. In some examples, the guide nucleic acid molecule is operably linked to a promoter or expression control element (examples of which are provided elsewhere in this application). In specific examples, the promoter is a minimal promoter, such as cytomegalovirus (CMV), human b-actin (hACTB), human elongation factor-la (hEFla), and cytomegalovirus early enhancer/chicken b-actin (CAG) promoters (e.g., the promoters described in Papadakis et al., Current Gene Therapy, 4:89-113, 2004; Damdindorj et al., PLOS ONE 9(8):e106472, 2014, both of which are incorporated by reference in their entirety). The vectors can include other elements, such as a gene encoding a selectable marker, such as an antibiotic, such as puromycin or hygromycin, or a detectable marker, such as GFP, another fluorophore, or a luciferase protein. Such vectors can include naturally occurring or non-naturally occurring nucleotides or ribonucleotides. Such vectors can be used in the methods, compositions, and kits provided herein.

The at least one reporter vector can include at least one reporter protein that is positioned downstream of a target sequence. Any suitable reporter protein can be used, such as a fluorescent protein, a bioluminescent protein, or any combination thereof. Exemplary reporter proteins include infrared-fluorescent proteins (IFPs), mRFP1, mCherry, mOrange, DsRed, dTomato (or tdTomato), mKO, tagRFP, EGFP, mEGFP, mOrange2, maple, tagRFP-T, firefly luciferase, renilla luciferase, and click beetle luciferase (e.g., US Pat. Pub. No. 2010/0122355, herein incorporated by reference in its entirety). In some examples, the at least one reporter protein can include about 1, about 2, about 3, about 4, or about 5 reporter proteins. In further examples, the at least one reporter protein can include about 1 to 5, about 1 to 4, about 1 to 3, about 1 to 2, about 2 to 5, about 3 to 5, about 4 to 5, or about 2 to 4, reporter proteins. In specific examples, the at least one reporter protein includes luciferase, mCherry, dTomato, or any combination thereof (e.g., a luciferase and mCherry combination or a luciferase and dTomato combination). The target sequence can be any target sequence of interest that is complementary to the crRNA or modified sgRNA (including dgRNA) of the gene activation vector.

The at least one gene activation vector includes at least one multiplex crRNA or multiplex sgRNA and at least one transcriptional activator protein. Multiplex crRNA and multiplex sgRNA are disclosed herein. Transcriptional activator proteins are also described herein, for example, VP64, p65, MyoD1, HSF1, RTA, SET7/9, or any combination thereof. In specific, non-limiting examples, the at least one transcriptional protein includes P65 and HSF1 (e.g., SEQ ID NO: 35).

### EXAMPLES

### Example 1

### Materials and Methods

### Mice

Gt(ROSA)26Sor^{tm1.1(CAG-cas9*,-EGFP)Fezh}/J (herein after Rosa26-Cas9 knockin or Rosa26-Cas9; Stock#024858) and C57BL/10ScSn-Dmd^{mdx}/J (herein after Mdx; Stock#001801) mice were obtained from Jackson Laboratory. Rosa26-Cas9 mice were mated with Mdx mice to generate Cas9^{+/-}-Mdx^{+/-}mice. Cas9^{+/-}Mdx^{+/-}mice were mated to generate Cas9Mdx mice. Both male and female mice 6-weeks to 4-month-old were used for this study.

### Plasmid Design and Construction

The sequence of MS2-P65-HSF1 (MPH) was cloned from the plasmid lenti_MS2-P65-HSF1_Hygro (Addgene 61426). The sequence of Spc5.12 promoter and CW3SL were directly synthesized by Gene Universal®. The EF1-MPH-CW3SL and Spc-MPH-CW3SL vectors were constructed by sub-cloning the EF1 or Spc5.12 promoter, MPH and CW3SL in the AAV backbone by using In-Fusion® cloning (Takara Bio). The mTGA constructs were synthesized by Gene Universal®. mTGA constructs were inserted into EF1-MPH-CW3SL and Spc-MPH-CW3SL vectors by the In-Fusion® cloning method to generate UtrnTriple AAV or UtrnTriple-crRNA AAV vectors. AAV dCas9 vector (AAV-Spc-dCas9) was constructed by replacing the nEF promoter of AAV-nEF-Cas9 (Liao, et al. (2017) Cell 171:1495-1507 e1415) with Spc5.12 promoter.

### AAV Production

AAV-DJ or AAV-Cas9 (AAV2 inverted terminal repeat (ITR) vectors pseudo-typed with AAV-DJ or AAV9 capsid) viral particles were generated following the procedures of the Gene Transfer Targeting and Therapeutics Core at the Salk Institute for Biological Studies. In brief, AAVpro HEK293T cells were maintained in 15 cm petri dishes with 20 ml complete DMEM (+10% FBS, GlutaMAX (100×), NEAA (100×)), and 30 plates were for high titer preparations. Cells were ˜70% confluent for transfection. The polyethylenimine transfection method was used to transiently transfect HEK293 cells. The cells were collected 72 hours after transfection and viruses were released to supernatant after 3 cycles of freeze-thaw. CsCl gradient centrifugation was used to purify the viruses followed by dialysis with 2 cycles of PBS and 1 cycle of 5% Sorbitol-PBS. The virus were then concentrated through an Amicon® Ultra-4 Centrifugal Filter Unit (Ultracel®-100K).

### Intramuscular Injection of AAV and Tibialis Anterior Muscle Collection and Section

Mice were anaesthetized with intraperitoneal injection of ketamine (100 mg/kg) and xylazine (10 mg/kg). The tibialis anterior muscles (TA) were collected and embedded with Tissue-Tek O.C.T. compound for cryosection according to the protocol of Wang and Kuang (Bio-Protocol 7: e2279, 2017). 10 μm-thick sections were collected on room temperature positive charged microscope slides. These slides were processed further for immunostaining.

### Immunostaining of Muscle Sections

Muscle sections were fixed with 4% paraformaldehyde. After washing with PBS and glycine, sections were blocked with blocking buffer (5% goat serum, 2% BSA, 0.2% triton X-100, and 0.1% sodium azide in PBS) for at least 30 min. Anti-utrophin (sc-15377 from Santa Cruz Biotechnology®) was diluted 200 times in blocking buffer and the sections were incubated with primary antibody overnight at 4° C. The next day, after washing with PBS, samples were incubated with Donkey anti-Rabbit IgG (H+L) (Alexa Fluor® 488, A-21206) and DAPI for 45 min at room temperature. Immunostaining images were captured with Zeiss® LSM 710 Laser Scanning Confocal Microscope.

### RNA Extraction and Real-time qPCR

Total RNA of muscles and myoblasts were extracted using Trizol® Reagent (Ambion®). The muscles and myofibers were homogenized by using EpiShear™M Probe Sonicator. RNA was treated with RNase-free DNase I to remove genomic DNA. The purity and concentration of total RNA were measured by SynergyTM H1 (BioTek®). cDNA was generated by reverse transcription using Maxima H Minus Reverse Transcriptase (ThermoFisher Scientific). SsoAdvanced™ Universal SYBR® Green Supermix (Bio-Rad) was used to carry out the qPCR analysis in CFX 384 Realtime System (Bio-Rad). The expression levels of respective genes were normalized to the housekeeping gene GADPH. Primers sequences were the same as in Liao, et al. (Cell 171:1495-1507 e1415, 2017).

### RNA-seq Analysis

Total RNA of isolated cells was collected at using the TRIzol® method. The Agilent 2200 TapeStationTM and the Invitrogen® Qubit® were used to evaluate the quality and quantity of RNA. RNA-Seq libraries will be constructed using the Illumina® Smart-Seq2® using Nextera® XT DNA Library Prep kit, and 2×150 bp pair-end sequencing is performed on an Illumina® HiSeq X™ Ten system. Raw reads were aligned to the mm10 genome using STAR [v2.5.3a] using default parameters. The number of reads were then uniquely aligned to RefSeq (available from The

National Center for Biotechnology Information (NCBI)) exons were quantified by HOMER [v4.9.1].

### Protein Extraction and Western Blot Analysis

Muscle samples were washed with PBS and homogenized with radioimmune precipitation assay buffer (50 mM Tris-HCl (pH 8.0), 150 mM NaCl, 1% NP-40, 0.5% sodium deoxycholate, and 0.1% SDS). Proteins (100 ug) were separated by 3-8% Criterion™M Tris-Acetate protein gel (Bio-Rad), electrotransferred onto a PVDF membrane (Millipore), and incubated with specific primary antibodies. Anti-utrophin (sc-15377 from Santa Cruz Biotechnology®) and anti-Gapdh (2188S from Cell Signaling) were diluted at a ratio of 1:1000 in 5% w/v nonfat dry milk.

Immunodetection was performed using SuperSignalTM West Pico PLUS Chemiluminescent Substrate (Thermo Scientific).

### Statistical Analysis

The data presented were taken from distinct samples with mean and standard deviation (SD). P-values were calculated using two-tailed unpaired Student's t-test. All analyses were performed with Prism 7 software. P-values <0.05 were considered to be statistically significant.

### Example 2

### Development of the Multiplex TGA (mTGA) System with Two dgRNAs

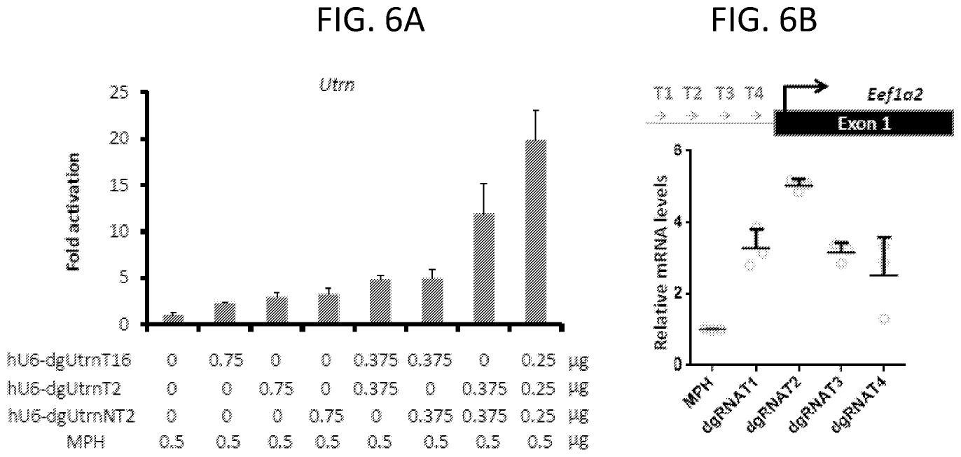

dgRNAs targeting different regions of the utrophin locus were screened for utrophin activation. It was observed that one gRNA (dgUtrnNT2, SEQ ID NO: 12) outperformed dgUtrnT2 and dgUtrnT16, which were the top efficiency gRNAs in the original screen (FIG. 5). dgUtrnNT2, dgUtrnT2 and dgUtrnT16 were selected for further testing to determine whether a synergistic effect could be achieved by transfecting combinations of gRNAs and MPH to N2a^{Cas9} cells. Activation of utrophin was enhanced when multiplexed dgRNAs were utilized without increasing the total dgRNA concentration. A mix of three dgRNAs (SEQ ID NOS: 12, 14, and 15) showed the strongest synergistic effect, with an 18-fold upregulation (7-fold higher than using a single dgRNA) (FIG. 6A).

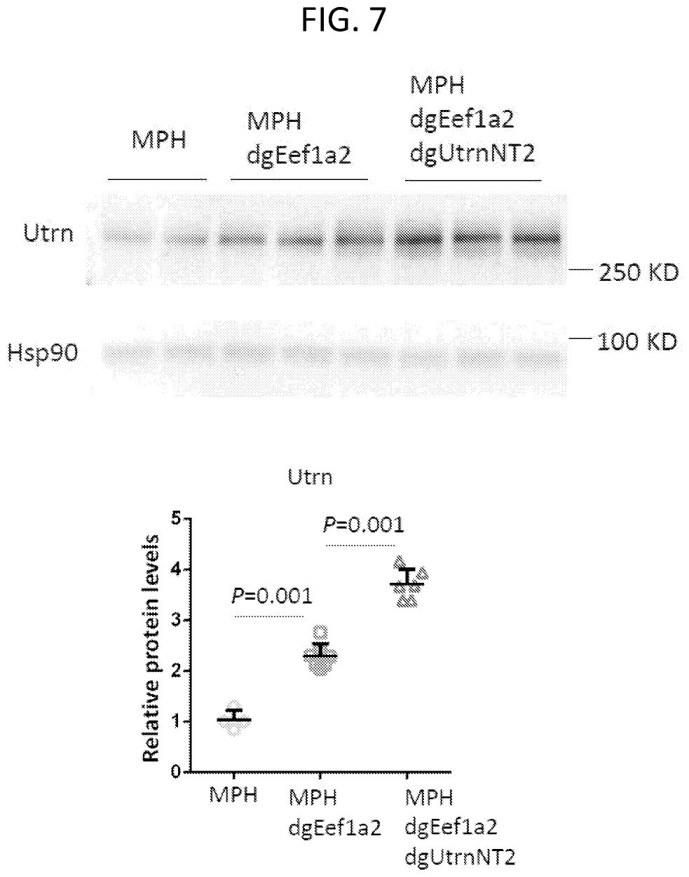

Eukaryotic translation elongation factor 1 alpha 2 (Eef1a2) is responsible for the translation of utrophin. Efficient dgRNAs to induce the expression of Eefla2 were identified (FIG. 6B), and it was investigated whether a duplex of Eef1a2 and utrophin dgRNAs could enhance the protein level of utrophin through enhancing transcription and translation simultaneously. dgEef1a2 (dgRNAT2) increased utrophin levels by 2.3-fold in N2aCas9 cells, and the duplex of dgEef1a2 and dgUtrnNT2 significantly enhanced the upregulation of utrophin 3.7-fold (FIG. 7). These results indicate that multiplexed gRNAs are able to enhance the efficiency of a TGA system.

Based on these findings, an mTGA system containing multiple utrophin and/or Eef1a2 dgRNAs and the MPH activation complex was developed in a single AAV vector for in vivo applications. Multiple modifications were made to develop the mTGA system. For example, to create space to insert additional dgRNAs within the same AAV vector, expression of the MPH transcriptional activation complex is driven by a shorter promoter. The original CAG promoter was replaced with either a ubiquitous promoter (EF1a) or a muscle-specific promoter (Spc5.12)

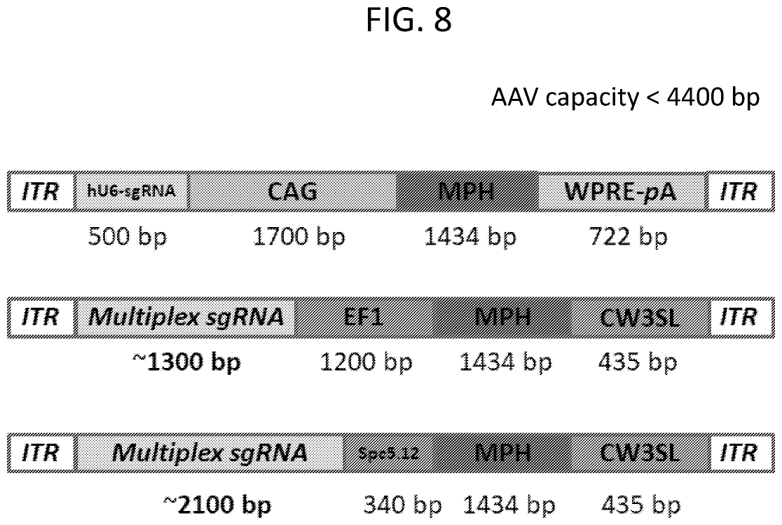

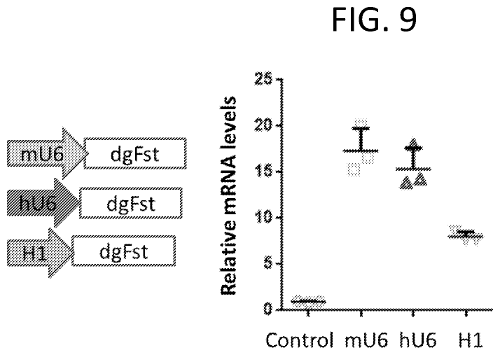

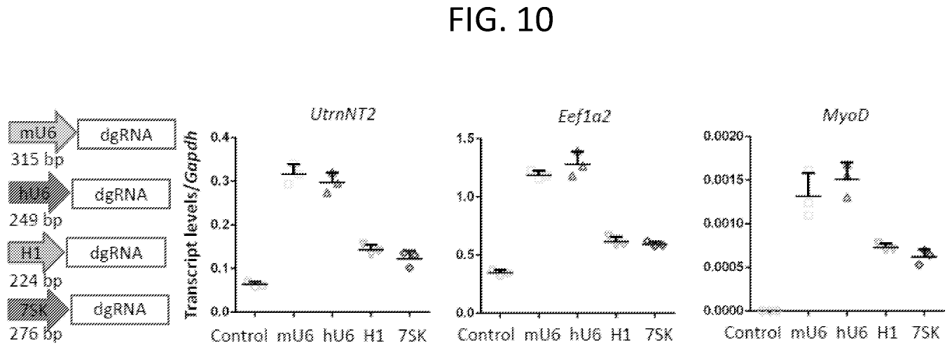

(FIG. 8). The WPRE-pA cassette was replaced with a shorter but equally efficient element, the CW3SL (Choi et al., Molecular brain, 7:17, 2014). In addition, adverse recombination events, such as truncation and rearrangement, are often observed in AAV vectors containing multiple repetitive fragments. Recombination can dramatically lower the efficiency of AAV and cause side-products with unwanted rearrangements. Two major sources of repetitive sequence are from the dgRNAs are their respective promoters. To address unwanted recombination, distinct RNA polymerase III promoters (hU6, mU6 and H1) were initially used to drive expression of different sgRNAs. hU6 and mU6 had about 2-fold higher activation efficiency than H1 (FIG. 9; see also, FIG. 10), thus hU6 and mU6 were selected for a mTGA system containing two sgRNAs.

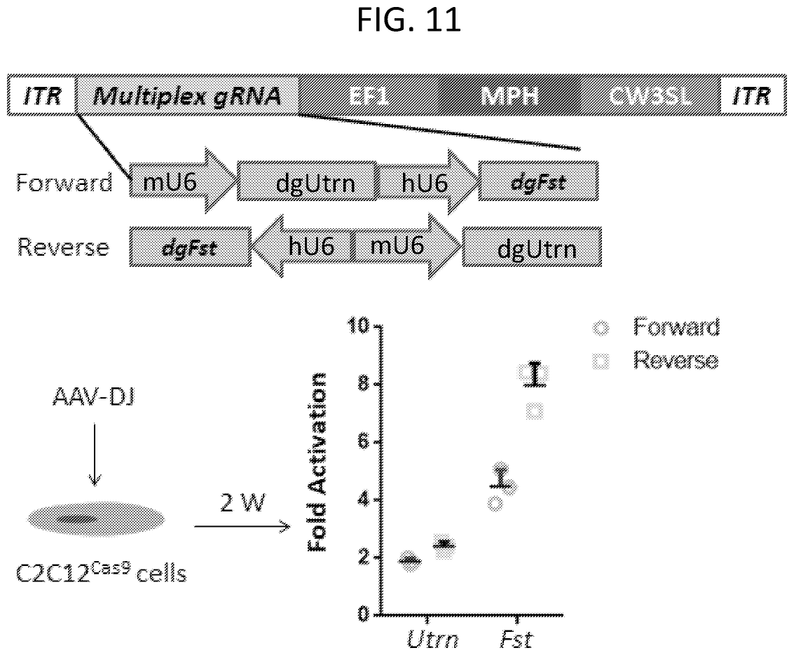

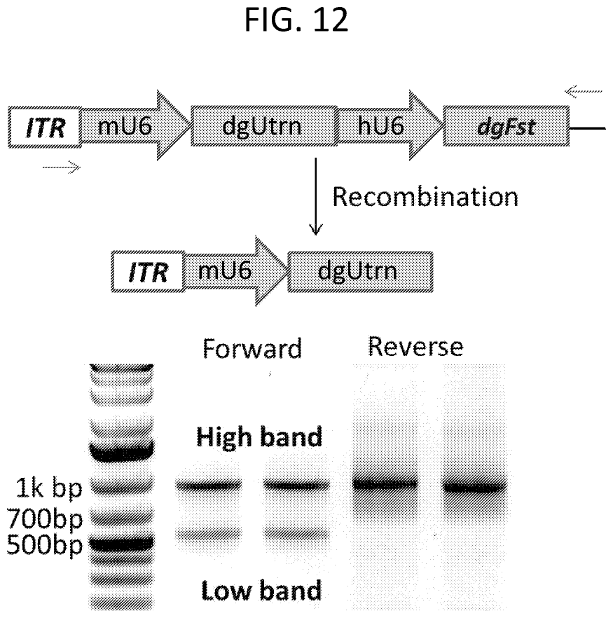

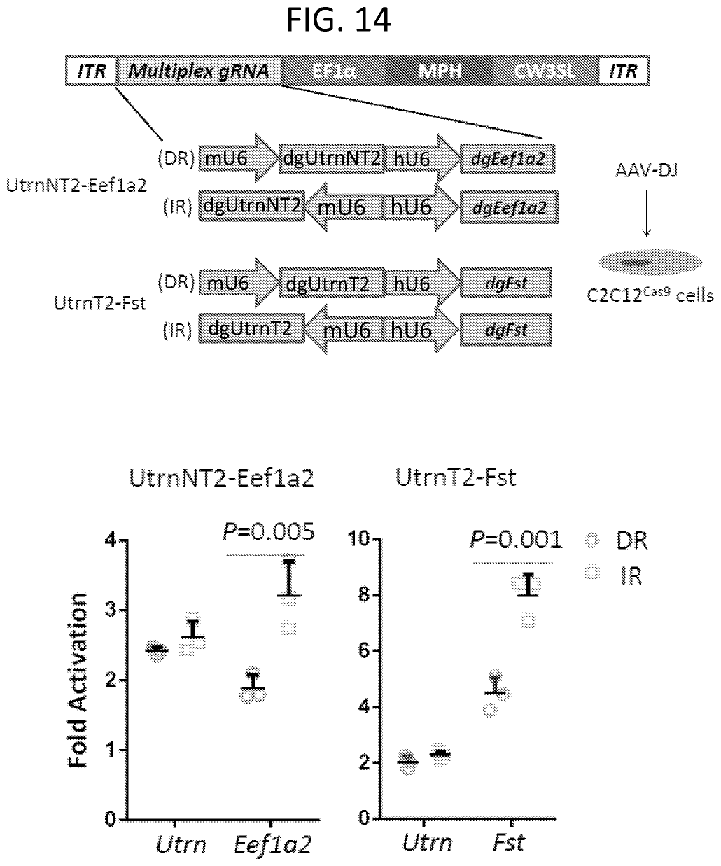

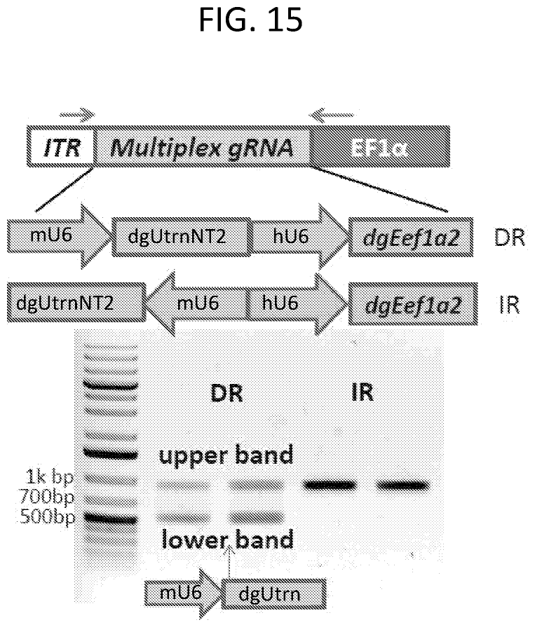

The activity of two mTGA systems containing two sgRNAs (in different orientations) were compared. It was found that the activity of targeted gene induction by the mTGA system with the inverted repeat (one sgRNA in forward orientation, one sgRNA in reverse orientation) is higher than the mTGA system with a direct repeat (both sgRNAs in forward orientation) (FIG. 11, see also, FIG. 14). It was also observed that the mTGA system with two sgRNAs in forward orientation tends to produce unwanted recombination, while such recombination did not occur in the mTGA system with an inverted repeat (FIGS. 12 and 13, see also, FIG. 15). The results demonstrate that the unwanted recombination of duo-dgRNAs can be reduced by the inverted repeat orientation.

### Example 3

### Duo mTGA System in vivo

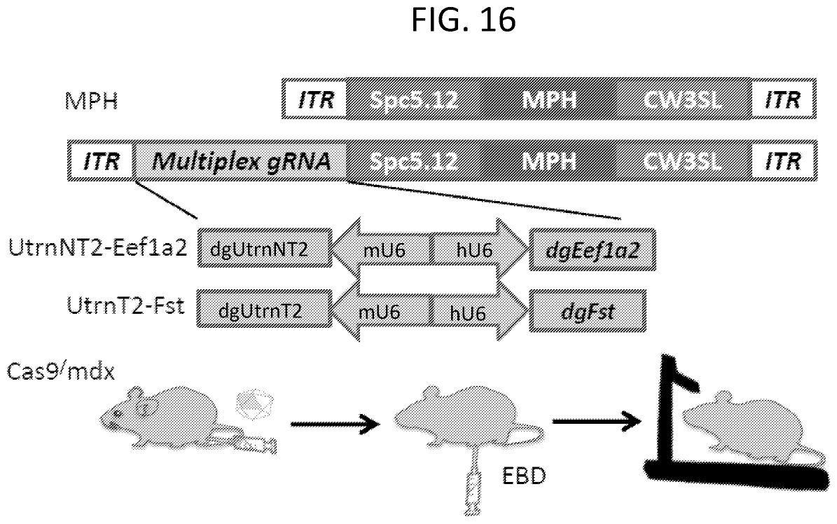

A skeletal muscle-specific duplex TGA system in which duo-dgRNAs oriented under inverted repeat with an MPH complex driven by the muscle-specific promoter Spc5.12 was designed (FIG. 16). The duplex TGA system was applied in vivo by intramuscular injections of 1×10¹¹ GC AAV9-dgUtrnT2-dgFst-MPH, AAV9-dgUtrnNT2-dgEef1a2-MPH or AAV9-MPH to tibialis anterior (TA) muscles of Cas9/mdx mice. The dgUtrnT2 and follistatin (Fst) dgRNA was applied individually to increase utrophin expression and to induce muscle hypertrophy, respectively (Liao et al., Cell 171:1495-1507, e1415, 2017). The duplex effect of dgUtrnT2/dgFst and dgUtrnNT2/dgEef1a2 on fragility of mdx muscles, which are sensitive to contraction-induced injuries, was investigated. Sarcolemmal integrity was monitored with Evans blue dye (EBD) assay 8-weeks after AAV injection (1×10¹¹ GC). Damaged myofibers accumulated EBD to produce red fluorescence.

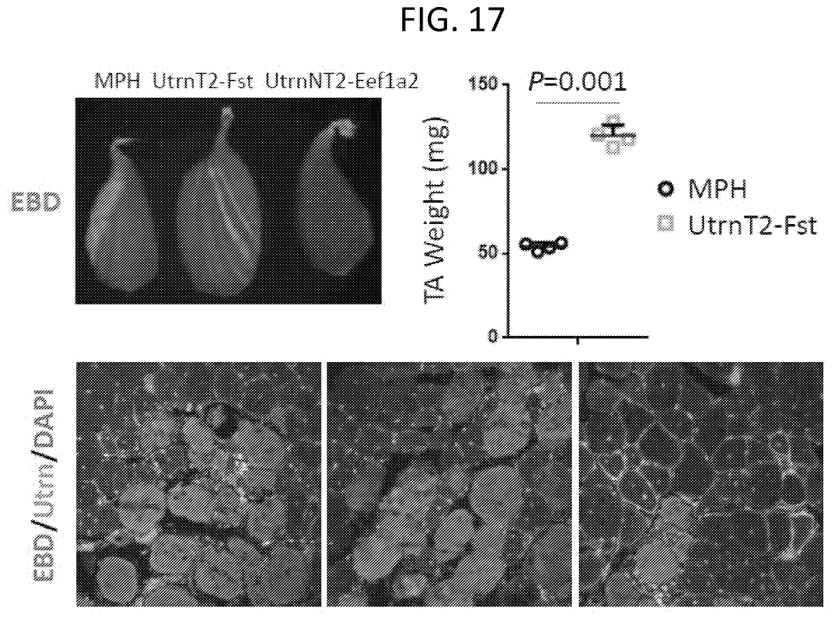

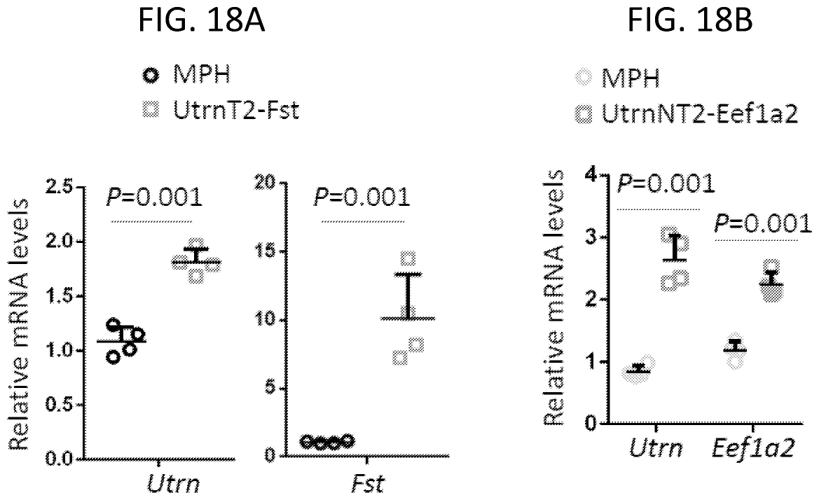

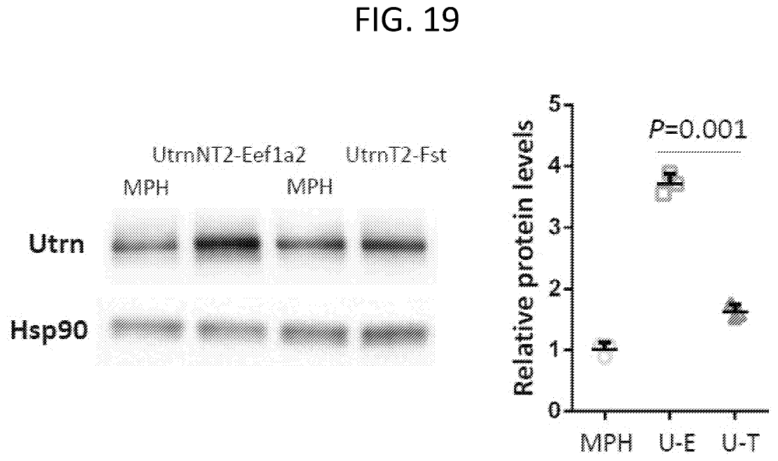

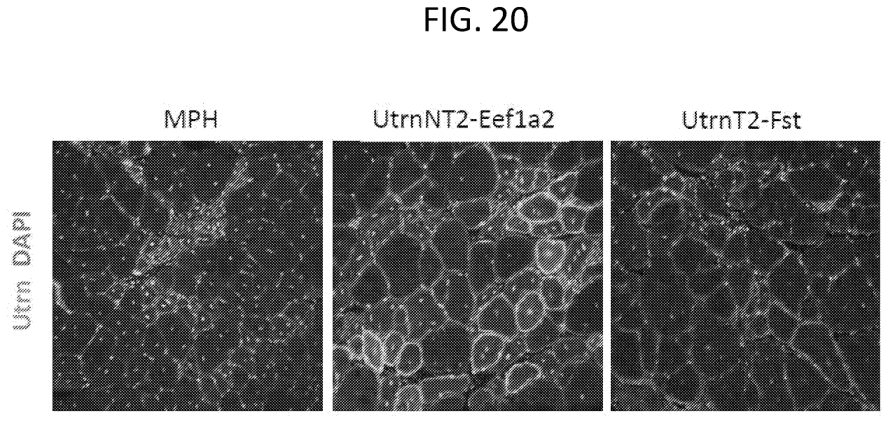

Extensive EBD uptake was observed in TA muscles with AAV9-MPH and AAV9-dgUtrnT2-dgFst-MPH injections (FIG. 17). In contrast, EBD uptake was greatly reduced in muscles treated with AAV9-dgUtrnNT2-dgEef1a2-MPH. The AAV9-dgUtrnT2-dgFst-MPH treatment induced muscle hypertrophy, but the increased muscle mass did not prevent muscle fragility (FIG. 17). Next, the expression of targeted genes was investigated. AAV9-dgUtrnT2-dgFst-MPH treatment increased expression of utrophin and Fst by 1.8-fold and 10-fold, respectively (FIG. 18A). The AAV9-dgUtrnNT2-dgEef1a2-MPH treatment increased the expression of utrophin and Eef1a2 by 2.6-fold and 2.2-fold, respectively (FIG. 18B). Protein levels of utrophin were also measured. The utrophin expression was upregulated 1.5-fold after AAV9-dgUtrnT2-dgFst-MPH treatment. In contrast, AAV9-dgUtrnNT2-dgEef1a2-MPH treatment boosted expression of utrophin 3.7-fold (FIG. 19). Immunostaining revealed a stronger utrophin signal in the sarcolemma of myofibers treated with AAV9-dgUtrNT2-dgEef1a2-MPH than with AAV9-dgUtrnT2-dgFst-MPH or AAV9-MPH (FIG. 20). The results show that the duplex mTGA system works efficiently in vivo to induce phenotypical changes. In addition, it is shown that the system can be designed to enhance the expression of utrophin to help prevent myofiber fragility.

### Example 4

### Development of mTGA System with Three dgRNAs

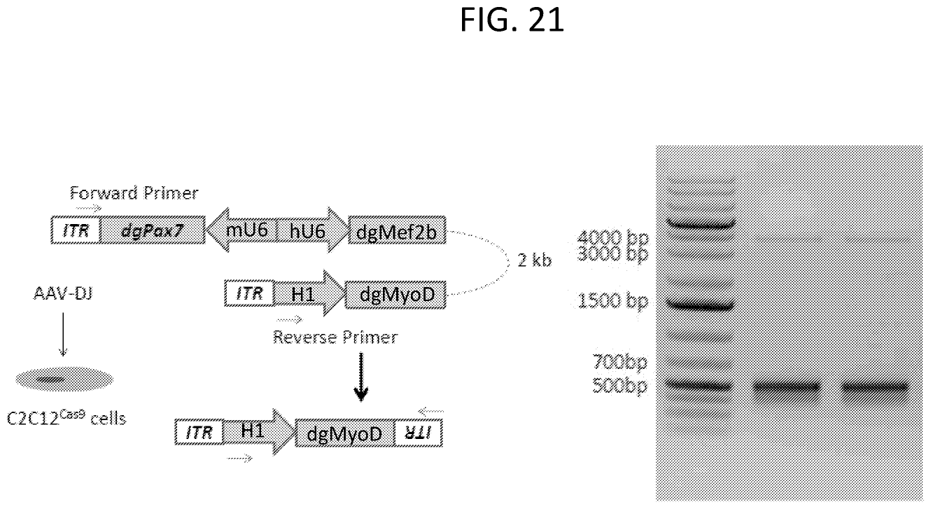

Although using two distinct RNA polymerase III promoters under inverted orientation helped reduce recombination when using two dgRNAs within the same AAV vector, additional challenges were faced in adding a third sgRNA. As shown in FIGS. 21 and 22, the addition of the third sgRNA comes with a direct repeat relative to one of the previously inverted sgRNAs, causing a significant truncation and inducing unwanted loss of dgRNA.

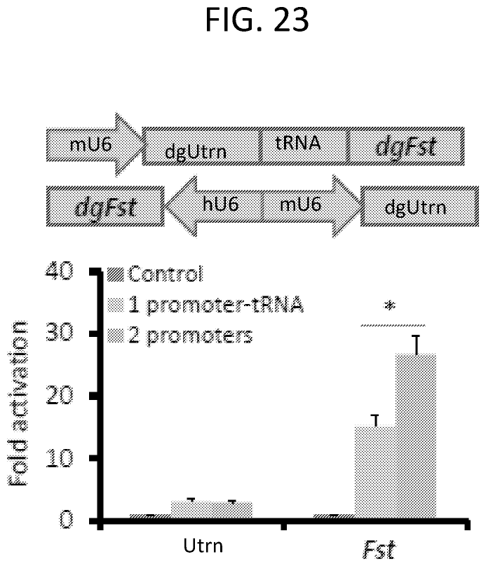

To address the issue, additional sgRNAs were incorporated using a technique that takes advantage of the endogenous tRNA-processing system. The activity of the sgRNA (dgFst) following a tRNA was found to be about half of that of the inverse construct containing dgFst directly driven by hU6 (FIG. 23).

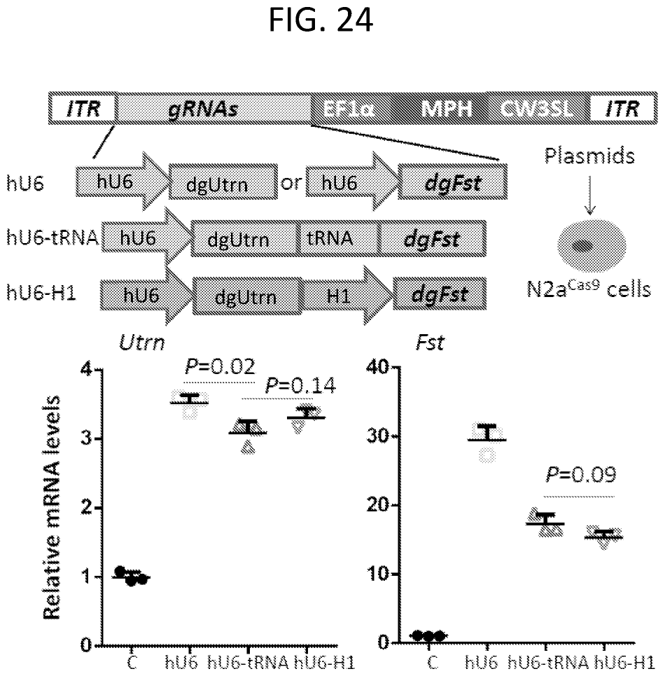

An hU6-dgUtrnNT2-tRNA-dgFst construct was also compared with a hU6-dgUtrnNT2-H1-dgFst construct, in which the third gRNA is driven by an HI promoter (FIG. 24). Due to the incomplete processing and maturation of gRNAs from tRNA-gRNA transcripts (Xu et al., Science advances 3:e1602814, 2017), the activation efficiency of the gRNA (dgUtrnNT2) upstream of tRNA and the gRNA (dgFst) downstream of tRNA was 10% and 44%, respectively, lower than that directly driven by hU6. There were no significant difference between gRNAs in the hU6-dgUtrnNT2-tRNA-dgFst construct and the hU6-dgUtrnNT2-H1-dgFst construct with non-viral plasmid transfection.

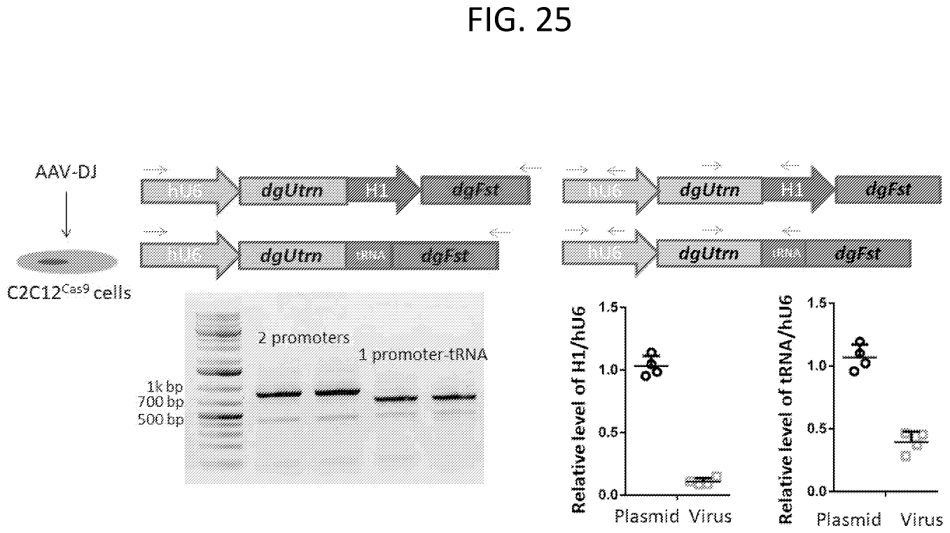

Considering the third sgRNA would have to be driven by the HI promoter (to avoid recombination), which also shows half the activation efficiency as compared to mU6 and hU6 promoter, decreased sgRNA activity following the tRNA was acceptable. The construct with a single promoter driving expression of two sgRNAs separated by a tRNA reduced adverse recombination events when the two sgRNA are both in forward orientation (FIG. 25). Thus, an mTGA system containing three sgRNAs was constructed.

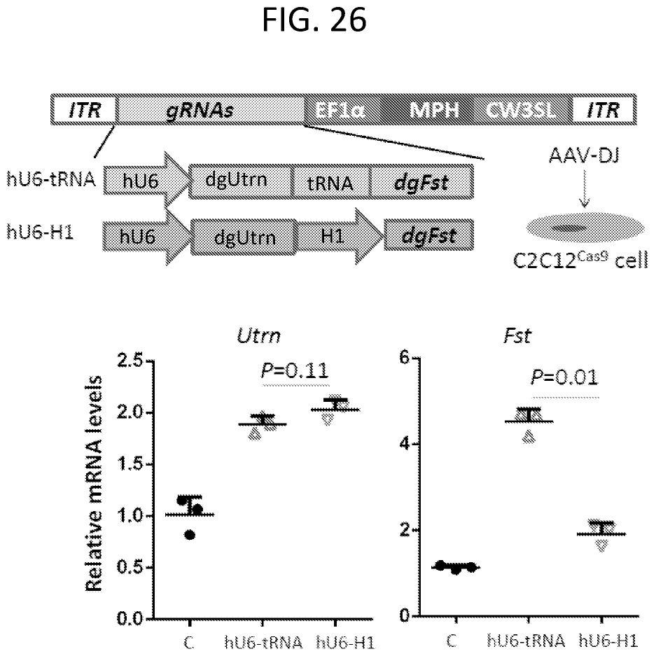

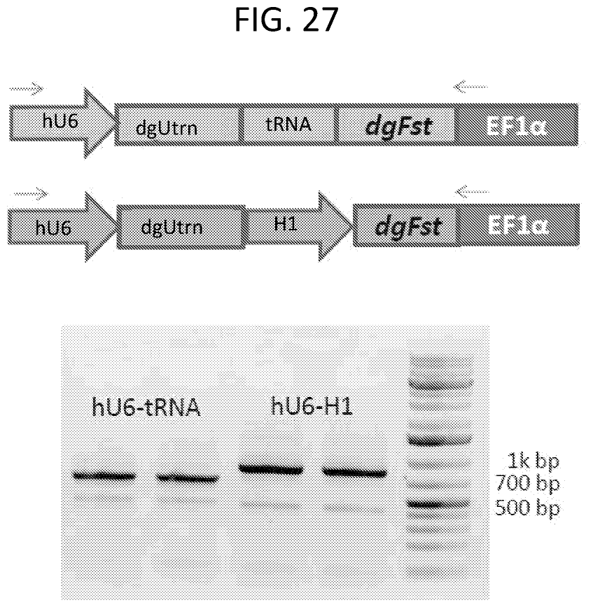

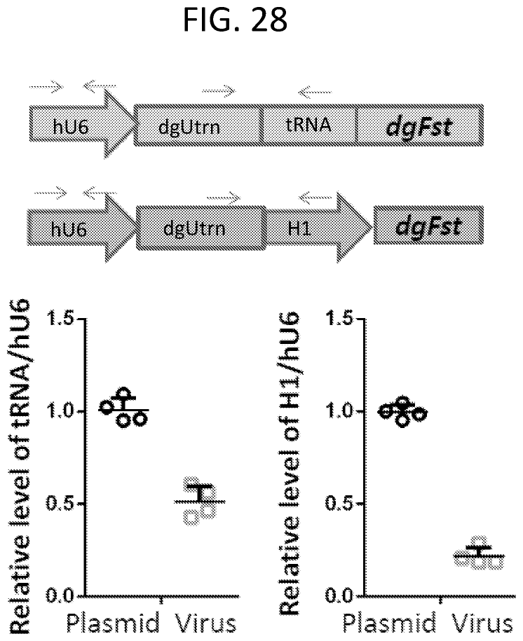

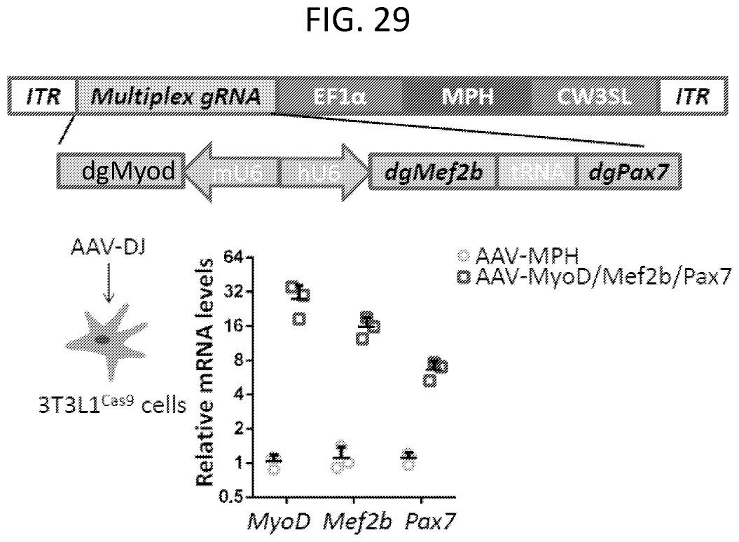

AAV containing the hU6-tRNA or hU6-H1 construct were tested in C2C12^{Cas9} cells with 1×10\hat{ }10 genome copies (GC) AAVDJ-hU6-dgUtrnNT2-tRNA-dgFst-MPH, AAVDJ-hU6-dgUtrnNT2-H1-dgFst-MPH or AAVDJ-MPH. The activation efficiency of dgUtrnNT2 was comparable between the hU6-tRNA and hU6-H1 constructs, however, the dgFst had 2.2-fold higher activation efficiency in the hU6-tRNA construct compared with the hU6-H1 construct (FIG. 26). Adverse recombination events were less in the hU6-tRNA construct than in the hU6-H1 construct (FIG. 27). The ratio of tRNA or H1 versus hU6 in plasmids and in AAV collected from the C2C12^{Cas9} cells were then quantified using qPCR. As tRNA or H1 was removed after recombination, the ratio reflects the recombination events that occurred during AAV production and infection. The ratio of tRNA versus hU6 in AAV was 51% of that in plasmid, while the ratio of H1 versus hU6 in AAV was 22% of that in plasmid, indicating a 59% (78% vs 49%) higher recombination events happened in the hU6-H1 construct compared with the hU6-tRNA construct (FIG. 28). Based on these observations, a mTGA system containing 3 gRNAs targeting MyoD, Mef2b and Pax7 was constructed. Efficient activation of MyOD, Mef2b and Pax7 in 3T3L1^{Cas9} cells was reported after treating of 1×10¹⁰ AAVDJ containing MPH only or the mTGA system (FIG. 29).

The mTGA system containing a combination of three tandem utrophin targeted sgRNAs (UtrnTriple) was developed and tested in vitro using non-viral transfection in N2^{Cas9} cells or using

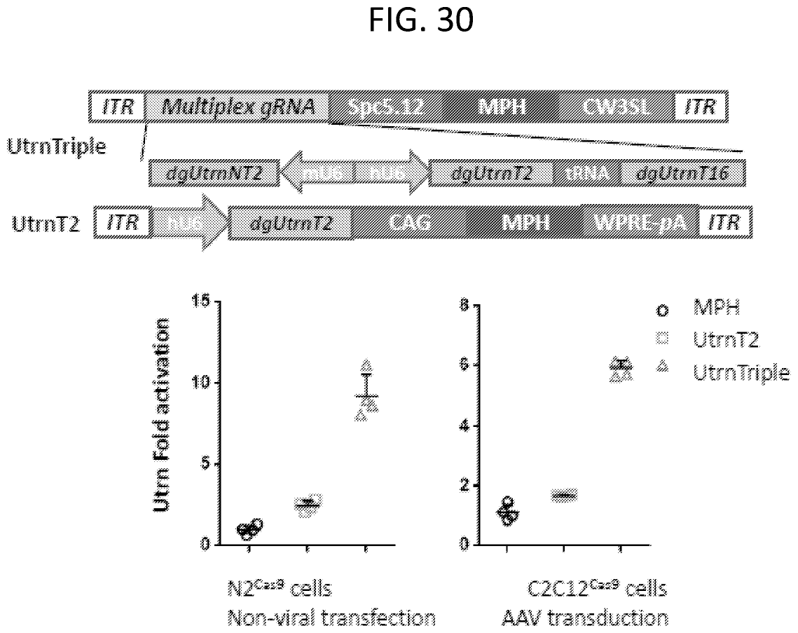

AAV (serotype DJ) transfection into C2C12^{Cas9} myoblasts. The controls included the AAV vector with a single utrophin dgRNA and MPH (UtrnT2), or MPH only. Activation of utrophin was higher using the mTGA as compared to either control (MPH only or the single-dgRNA TGA system) (FIG. 30).

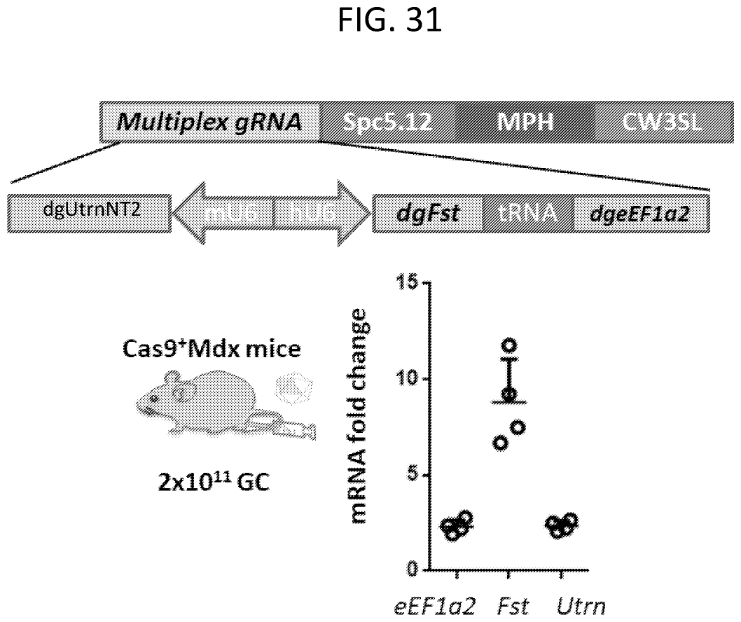

It was also confirmed that the mTGA system containing three dgRNAs activates the expression of multiple target genes in tibialis anterior (TA) muscles of Cas9+Mdx mice (FIG. 31).

### Example 5

### Development of mTGA System with Four dgRNAs

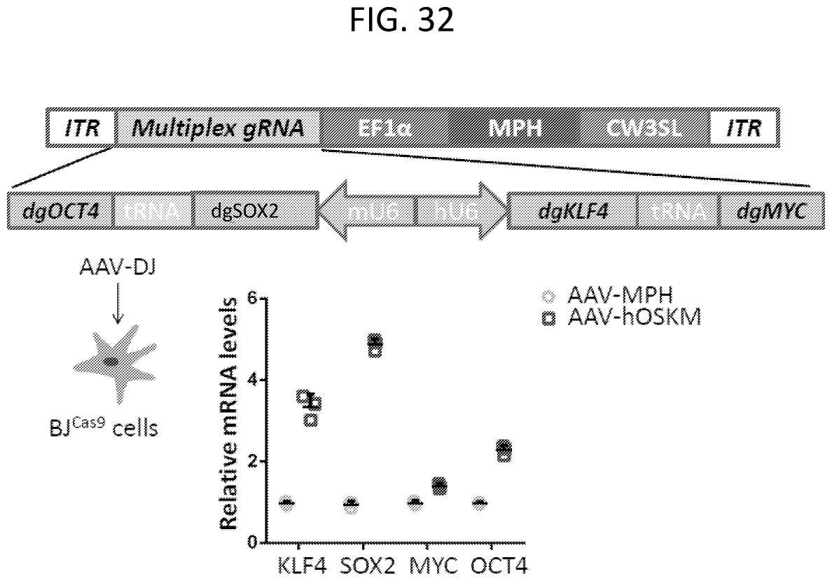

The mTGA system was expanded to contain four gRNAs. The 3^{rd} and the 4^{th} gRNA were driven by mU6 and hU6 after tRNA processing (FIG. 32). Two different tRNAs (from yeast and corn) were chosen to minimize repetitive sequences (Xie et al., PNAS, 112:3570-3575, 2015; Zhang et al., Nature Communications, 10:1053, 2019). The mTGA system was used to activate expression of OCT4, SOX2, KLF4 and c-MYC in BJ^{Cas9} cells by treating of 1×10¹⁰ AAVDJ containing MPH only or the mTGA system (FIG. 32). The results show that the AAV-mediated mTGA system works efficiently to activate at least four genes.

### Example 6

### UtrnTriple mTGA System in vivo

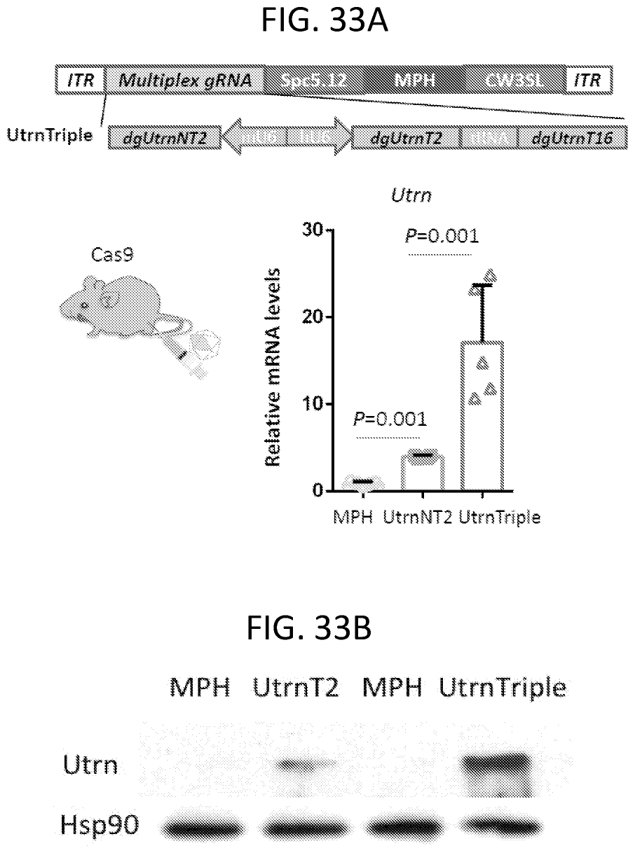

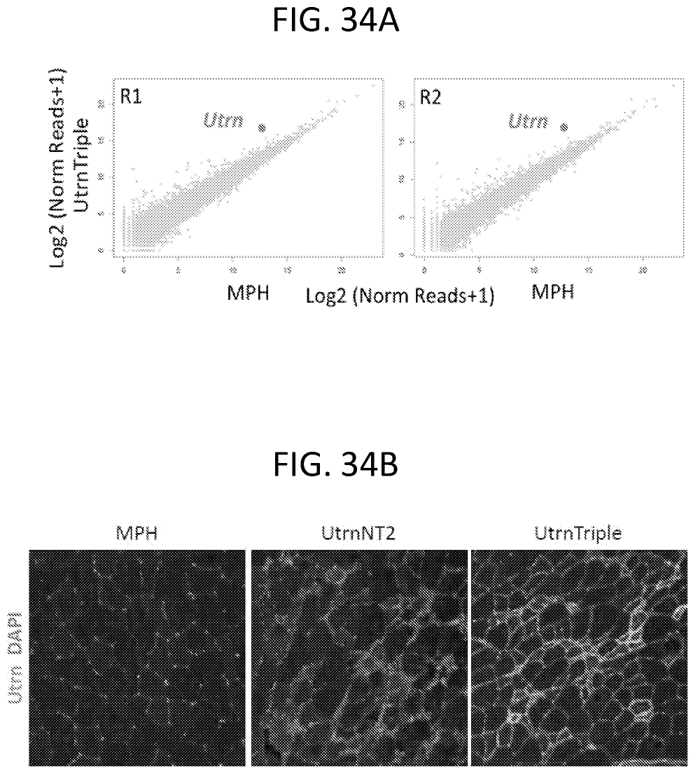

The mTGA system was tested in vivo by intramuscular injections of 2×10¹¹ vg AAV (serotype 9) containing MPH only (AAV-MPH), the TGA system (one utrophin sgRNA, AAV-UtrnT2, see US Pub. No. US-2021-0102206-A1), or the mTGA system (triple utrophin sgRNAs, AAV-UtrnTriple) into TA muscles of Cas9-expressing mice. Two months after AAV injection, the expression of utrophin was increased by up to 24-fold (average increase of 16-fold) in muscles injected with the mTGA system (FIG. 33A). In contrast, the average level of increase was only 2.5-fold for the original TGA system (UtrnT2). RNA-seq analysis was also performed for an unbiased analysis of utrophin expression. The norm reads of utrophin was ˜16-fold higher in muscles treated with the mTGA system as compared with MPH only (FIG. 33B). It was also verified that levels of utrophin protein were higher using the mTGA system (FIG. 34A). Immunostaining using antibodies against utrophin showed an increase of sarcolemmal localization in UtrnTriple-treated muscles compared with UtrnT2-treated TA muscles (FIG. 34B).

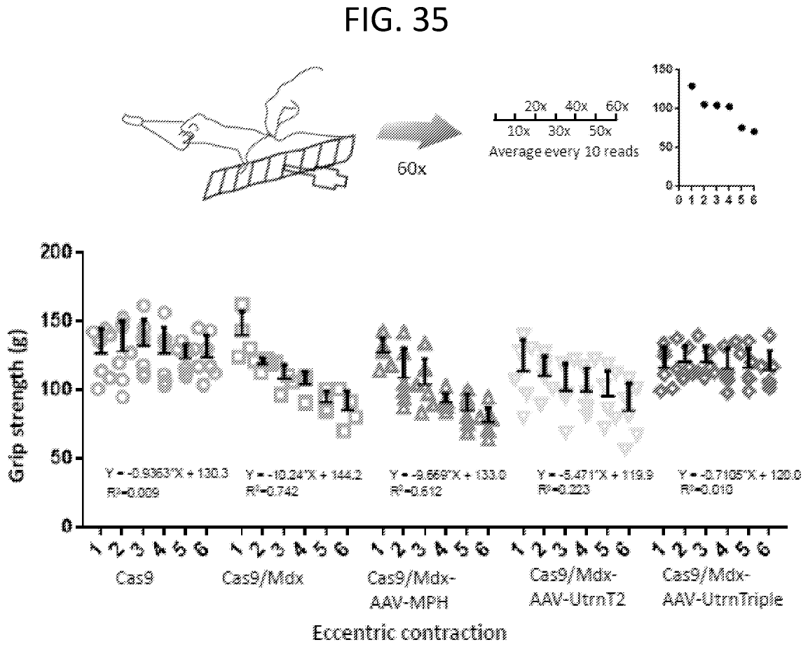

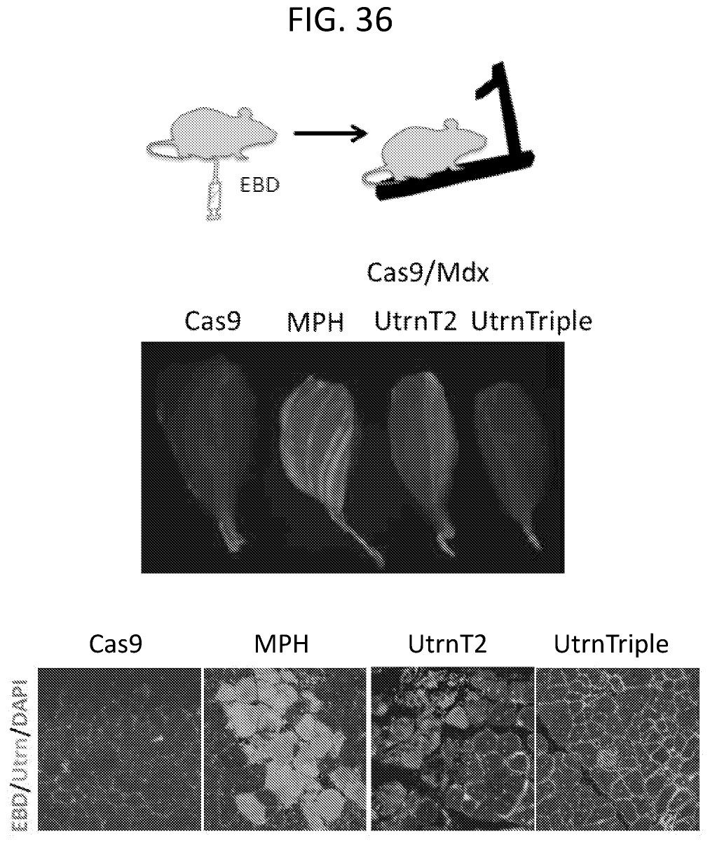

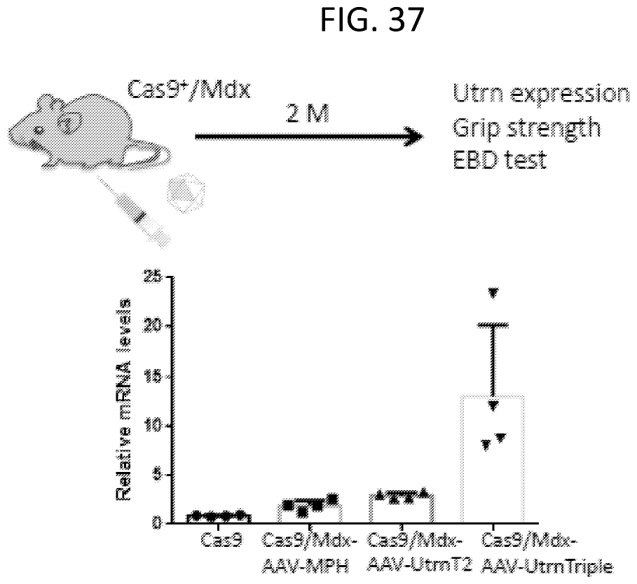

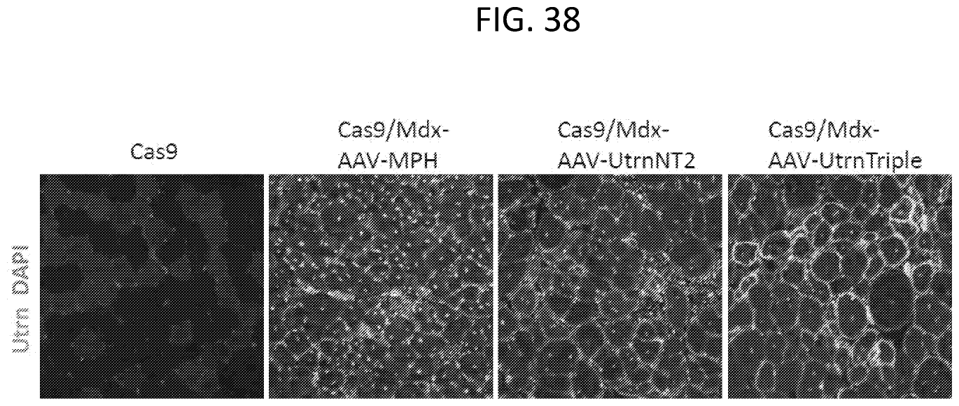

The new mTGA system was further tested in vivo by intramuscular injections of 2×10¹¹ vg AAV-MPH, AAV-UtrnT2 or AAV-UtrnTriple into TA and gastrocnemius (GA) muscles of Cas9/Mdx mice. Grip strength and the uptake of Evans blue dye (EBD) was evaluated two months after AAV injection. Grip strength tests were repeated 60 times continuously for each mouse. The reads of every 10 tests were averaged. The grip strength of Cas9 mice were found to be constant with the continuous test. In contrast, the grip strength of Mdx/Cas9 mice and Mdx mice were decreased in a linear regression pattern with a slope of about −10 (FIG. 35). While TGA treatment slowed the decrease trend with a slope of −5, mTGA treatment rescued the decreased grip strength (FIG. 35). Sarcolemmal integrity was also monitored by the uptake of EBD, which accumulates in damaged cells. The data show extensive EBD uptake in Mdx mice with AAV-MPH and AAV-UtrnT2 injection (FIG. 36). In contrast, EBD uptake is greatly reduced in AAV-UtrnTriple treated mice (FIG. 36). The expression of utrophin in TA muscles with one utrophin gRNA or multiplex utrophin gRNAs was also measured. There was significant activation of utrophin in mTGA treated mice as compared to the other samples (FIGS. 37 and 38).

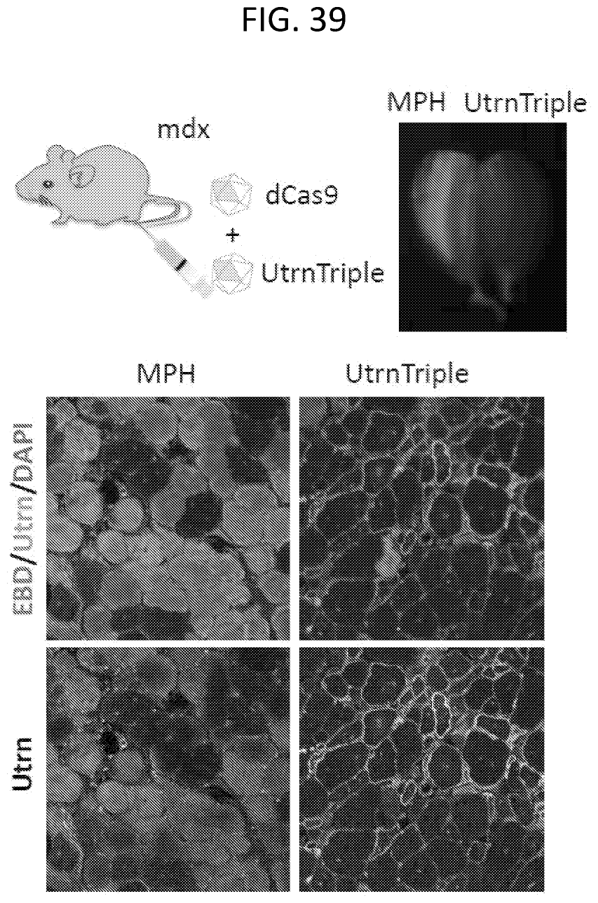

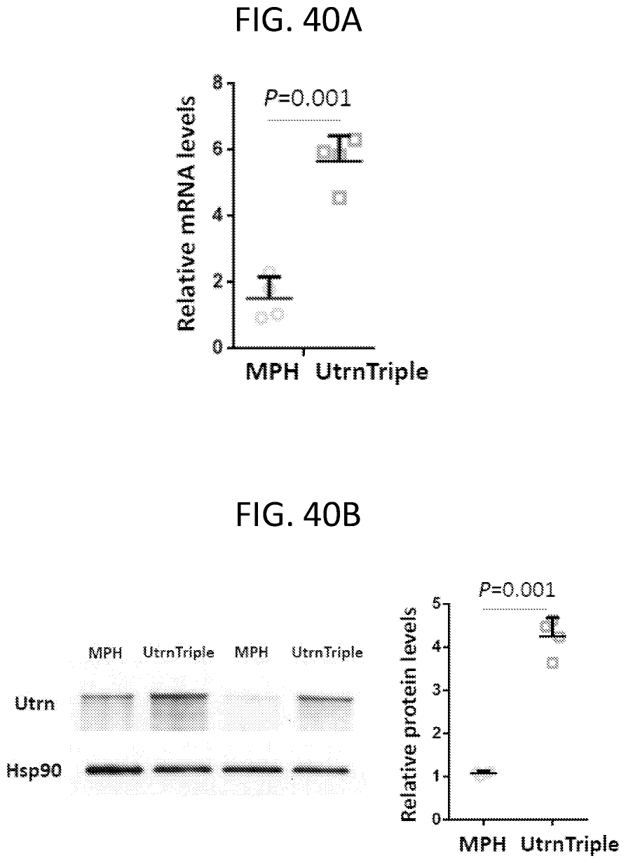

The mTGA system was tested in wildtype (WT) mdx mice using a dual-AAV system by injecting 1×10¹¹ GC AAV9-dCas9 and AAV9-UtrnTriple into the TA muscle of one side of the mouse (FIG. 39). The contralateral TA muscle control was injected with AAV9-dCas9 and AAV9-MPH. The sarcolemmal integrity was evaluated by uptake of EBD two months after treatment (FIG. 39). Extensive EBD uptake was found in the control treatment. In contrast, the EBD uptake is significantly alleviated by mTGA treatment. In addition, the immunostaining confirmed efficient activation of utrophin (FIG. 39). The expression of utrophin was quantified by qPCR and western blot. The mRNA level of utrophin was increased by 4.6-fold in the TA muscles treated with mTGA system compared to control legs (FIG. 40A). Western blots showed that the protein level of Utrn was significantly elevated by 4-fold (FIG. 40B). Thus, the disclosed mTGA system can be utilized as a treatment for DMD.

### Example 7

### Multiplexed gRNAs Synergistically Enhance Epigenetic Modifications

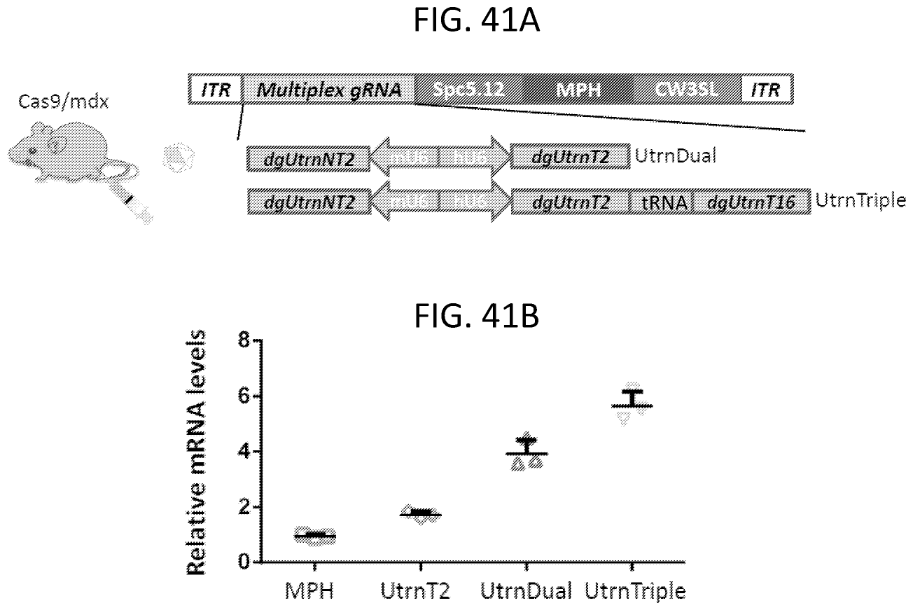

The TGA system can modify histone modifications near the targeted genomic locus (Liao et al., Cell 171:1495-1507, e1415, 2017). To identify histone modifications after mTGA treatment, TA muscles of Cas9/mdx mice were injected with 1×10¹¹ GC AAV9-MPH, AAV9-hU6-dgUtrnT2-MPH, AAV9-UtrnDual or AAV9-UtrnTriple (FIG. 41A). The mRNA level of utrophin was marginally increased by only dgUtrnT2 2-month after AAV injection (FIG. 41B). In contrast, its level was increased 4-fold by AAV9-UtrnDual and 5.5-fold by AAV9-UtrnTriple.

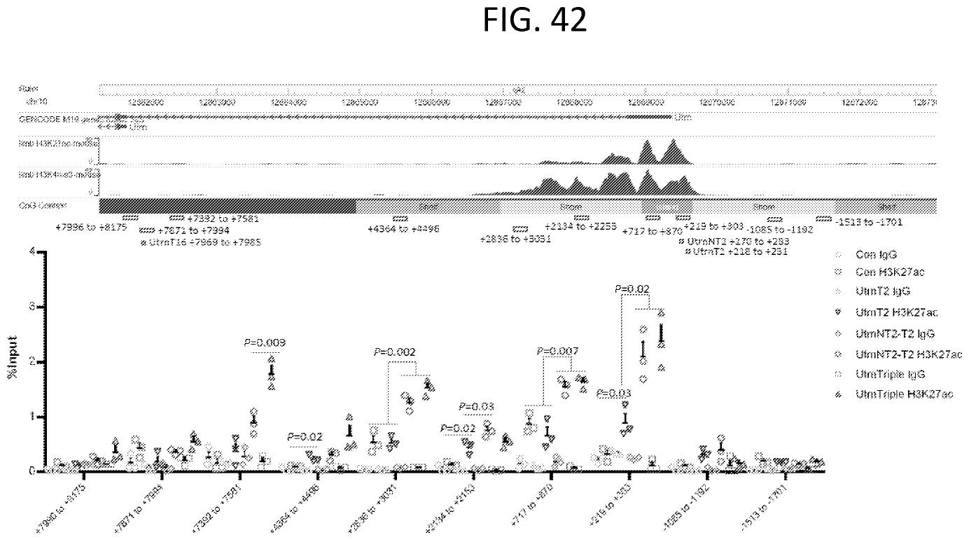

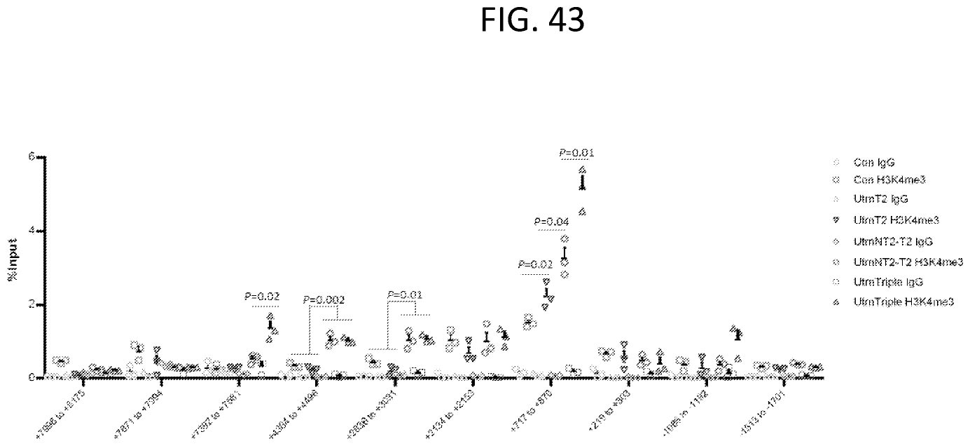

Chromatin-immunoprecipitation (ChIP) qRT-PCR of the TA muscle samples was performed. H3K4me3 and H3K27ac epigenetic marks, which are typically associated with transcriptionally active genes, were enriched at the target locus of AAV9-hU6-dgUtrnT2-MPH injected mice, compared to AAV9-MPH controls (FIGS. 42 and 43). Intriguingly, AAV9-UtrnDual and AAV9-UtrnTriple not only enhanced the enrichment of H3K4me3 and H3K27ac marks, but also extended epigenetic changes compared to AAV9-hU6-dgUtmT2-MPH. AAV9-UtrnTriple further changed the epigenetic marks around UtrnT16 compared to AAV9-UtrnDual. The data shows that the mTGA system synergistically enhances epigenetic changes around the target sites.

### Example 8

### Endurance of Utrophin Activation Elicited by Mtga System

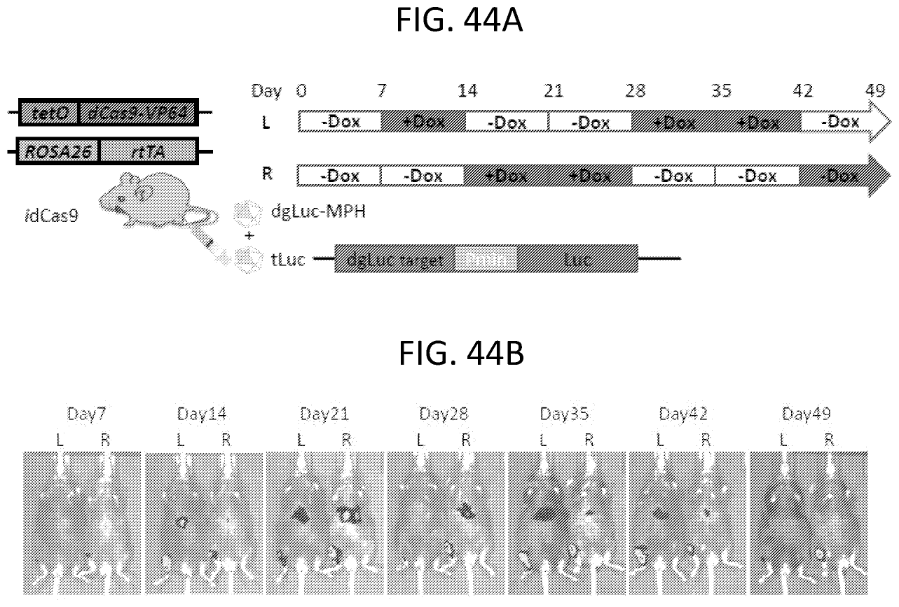

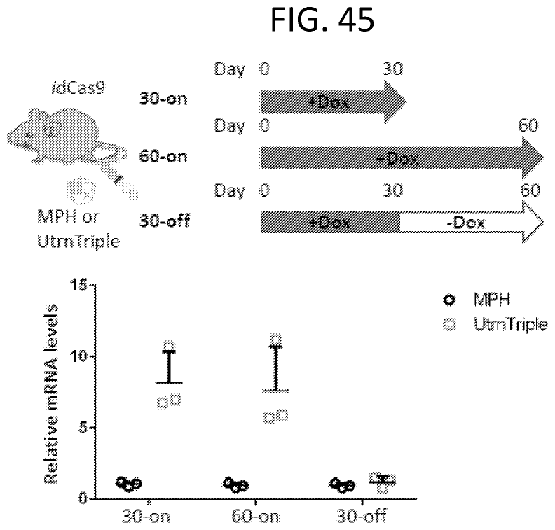

Although the mTGA system induced strong epigenetic changes, it was unknown whether long-term gene activation can be achieved with short-term expression of the system. To investigate, a mouse line (idCas9) carrying a tetO-driven dCas9 plus the reverse tetracycline transactivator (rtTA) was generated; allowing regulation of the expression of dCas9 through doxycycline (Dox) administration (FIG. 44A). TA muscles of the idCas9 mice were co-injected with AAV containing a luciferase reporter in which luciferase was placed downstream of a dgRNA (dgLuc) binding site and AAV containing a dgLuc-CAG-MPH sequence. Then, Dox water (1 mg/ml) was added and removed at an interval of 1-week or 2-weeks. The luciferase signal was strikingly induced 1-week after Dox administration, and turned back to the basal level 2-week after Dox removal (FIG. 44B). As dCas9 was required for the activation of luciferase, the data verifies that the expression of dCas9 was regulated by Dox administration in idCas9 mice. Next, endogenous activation of utrophin in idCas9 mice, which were injected with 1×10¹¹ GC AAV9-UtrnTriple or AAV9-MPH, was investigated. The expression of utrophin was increased by around 8-fold after a continuous 30-day or 60-day Dox administration (FIG. 45). In contrast, no overexpression of utrophin was found after a 30-day Dox withdrawal. These data demonstate that the mTGA system is required for gene activation.

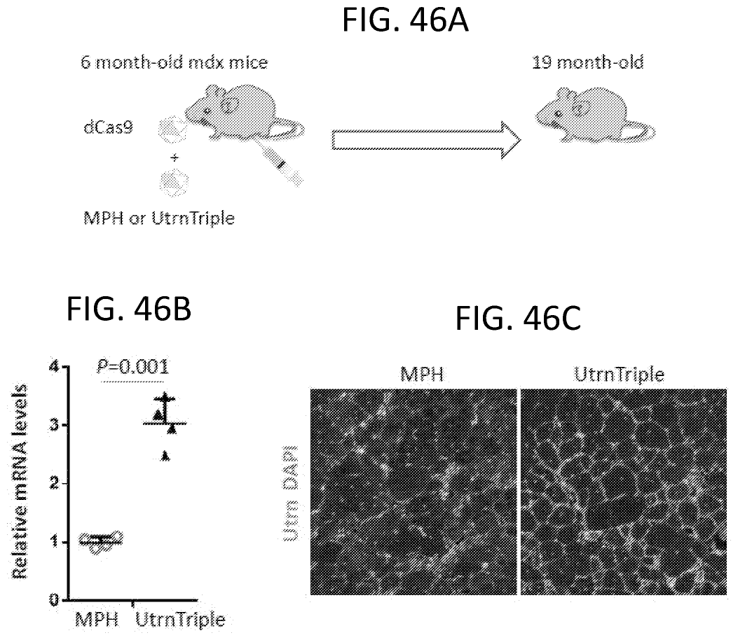

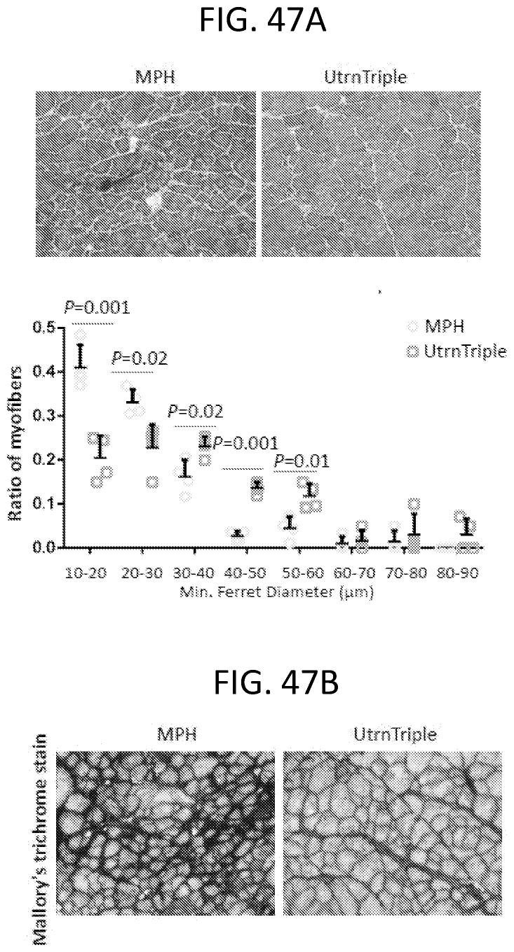

Persistent transgene expression has been reported in human skeletal muscle 10 years after injection of AAV carrying the transgene (Buchlis et al., Blood 119:3038-3041, 2012). To track the endurance of AAV-mediated mTGA system, TA muscles of 6-month-old mdx mice were co-injected with 1×10¹¹ GC AAV9-dCas9 and AAV9-UtrTriple or AAV9-MPH (FIG. 46A). The muscle samples were collected 13-months later, and a 3-fold increase of utrophin was found in samples treated with the mTGA system (FIG. 46B). Immunostaining verified the efficient activation of utrophin (FIG. 46C). H&E staining and Mallory's trichrome staining were utilized to evaluate the histopathological phenotypes of mdx muscles. H&E staining showed that the muscle interstitial space was larger and the myofiber size was smaller in control treatment compared with mTGA treatment (FIG. 47A). In addition, Mallory's trichrome staining showed that the mTGA-treated muscles had less fibrosis compared to control muscles (FIG. 47B). Thus, the AAV-mediated mTGA system has a long-lasting effect in gene activation and pathological phenotype amelioration.

### Example 9

### Enhancing mTGA Efficiency by Optimizing gRNA Combinations

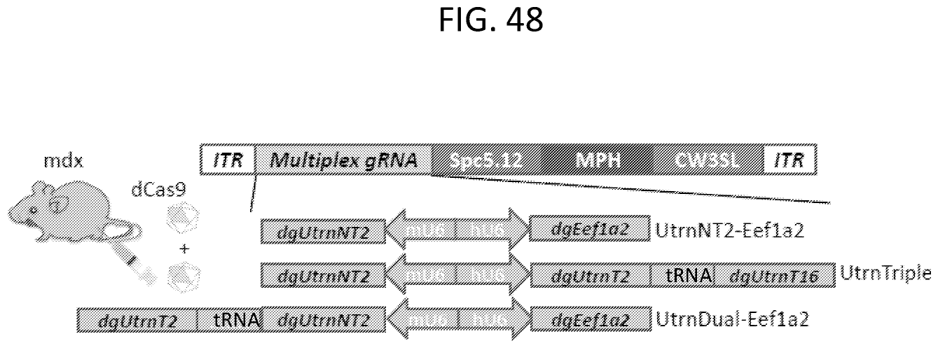

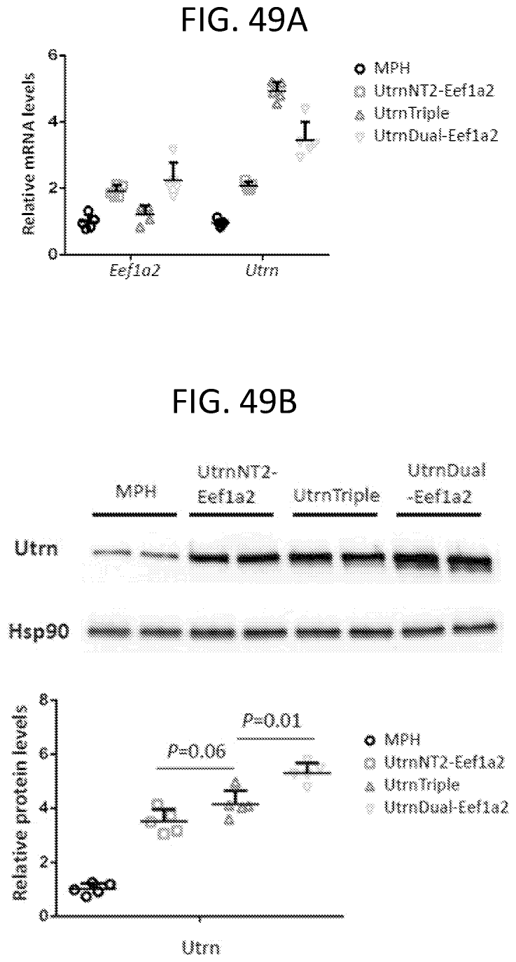

The combination of gRNAs to enhance the expression of utrophin was optimized. As dgUtrnNT2-dgUtrnT2 (UtrnDual) and dgUtrNT2-dgUtrT2-dgUtrnT16 (UtrnTriple) similarly changed the histone modifications of utrophin promoter, an AAV9-UtrnDual-Eef1a2 was generated to simultaneously enhance the transcription and translation of utrophin and compared it with AAV9-UtrnTriple and AAV9-UtrnNT2-Eef1a2 (FIG. 48). Two months after a dual-AAV injection (1×10¹¹ GC) into TA muscles of mdx mice, AAV9-UtrnDual-Eef1a2/AAV9-dCas9 treatment increased expression of Eef1a2 by 2.2-fold and the expression of utrophin by 3.5-fold (FIG. 49A). In contrast, AAV9-UtrnNT2-Eef1a2/AAV9-dCas9 increased the expression of Eef1a2 and utrophin by 1.9-fold and 2-fold, respectively, and AAV9-UtrnTriple/AAV9-dCas9 upregulated the expression of utrophin by 4.9-fold without changing the expression of Eef1a2 (FIG. 49A). Intriguingly, AAV9-UtrnDual-Eef1a2/AAV9-dCas9 treatment enhanced the utrophin protein by 5.3-fold, improving the upregulation of utrophin protein by 27% compared to AAV9-UtrNT2-Eef1a2/AAV9-dCas9 and AAV9-UtrnTriple/AAV9-dCas9 treatments (FIG. 49B).

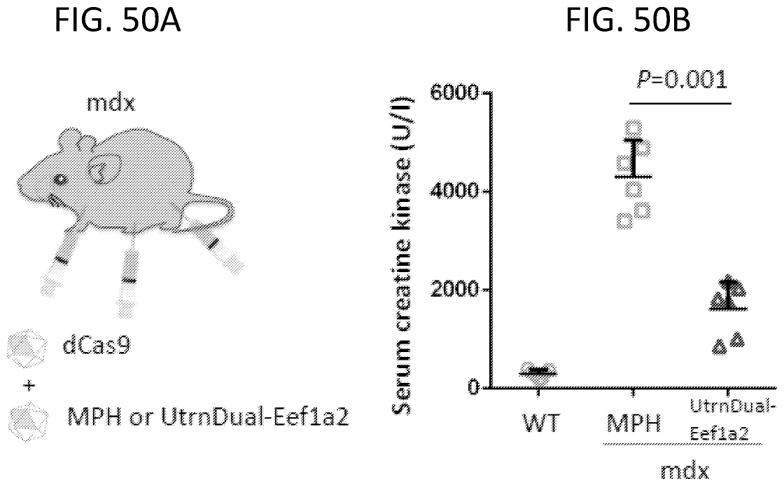

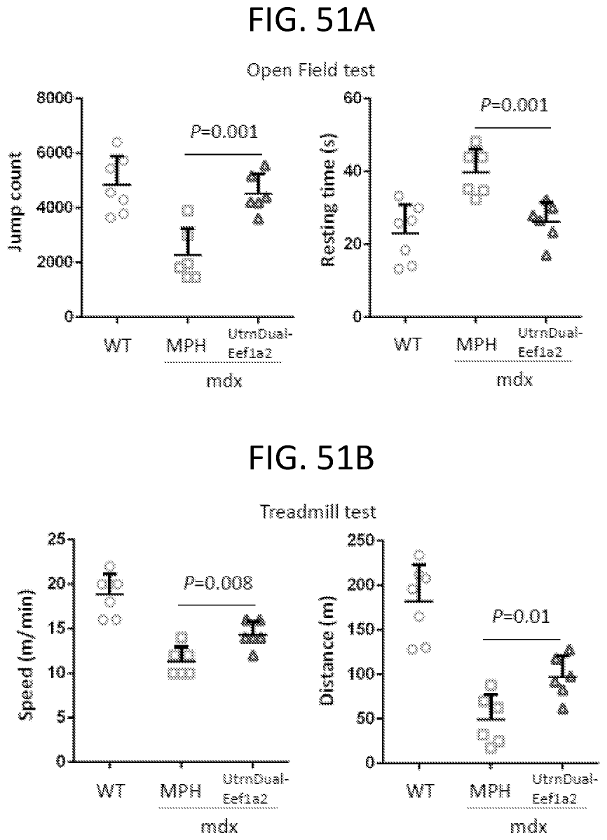

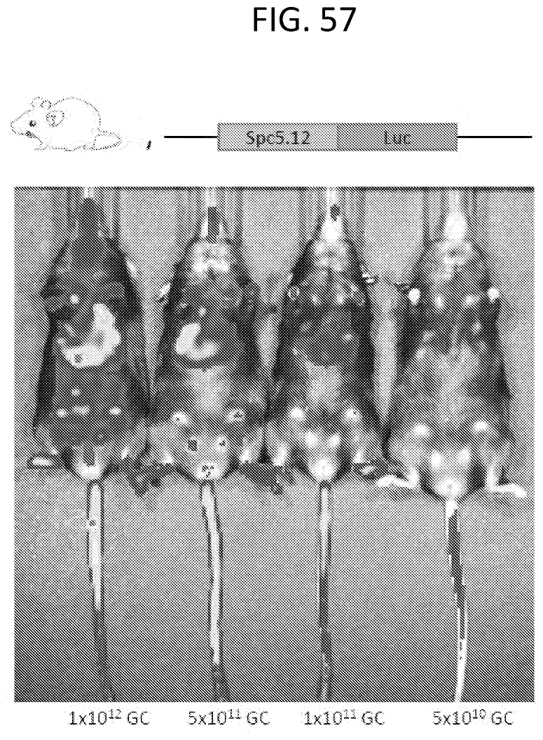

The optimized mTGA system containing AAV9-UtrnDual-Eef1a2 and AAV9-dCas9 was also used to treat adult mdx mice. Treatment of the whole body through tail vein injection was considered, however, use of a luciferase reporter (AAV9-Spc5.12-Luc) to trace the distribution of AAV after tail vein injection revealed that the AAV did not efficiently enter into muscle cells even at a high AAV titer (1×1012 GC; FIG. 57). Thus, instead of using tail vein injection, intramuscular injection of the dual-AAV system into multiple muscles of 2-month-old mdx mice (with a titer according to the muscle size), including TA muscles (1×10¹¹ GC), GA muscles (2×10¹¹ GC), Quadriceps femoris muscles (2×10¹¹ GC), Deltoid muscles (5×10¹⁰ GC), Triceps brachii muscles (5×10¹⁰ GC), Spinotrapezius muscles (1×10¹¹ GC) (FIG. 50A). Two months after AAV treatment, the activity of serum creatine kinase decreased by 3-fold in mice treated with mTGA system compared with mice treated with AAV9-MPH/AAV9-dCas9 (FIG. 50B). In an open field test, control mdx mice had a lower jump count and more resting time as compared to WT mice. mTGA treatment rescued the decreased activity of mdx mice (FIG. 51A). A treadmill test also revealed that mTGA treatment improved the speed and endurance of treated mdx mice as compared to control mdx mice (FIG. 51B).

### Example 10

### Development of Multiplex crRNA mTGA Constructs

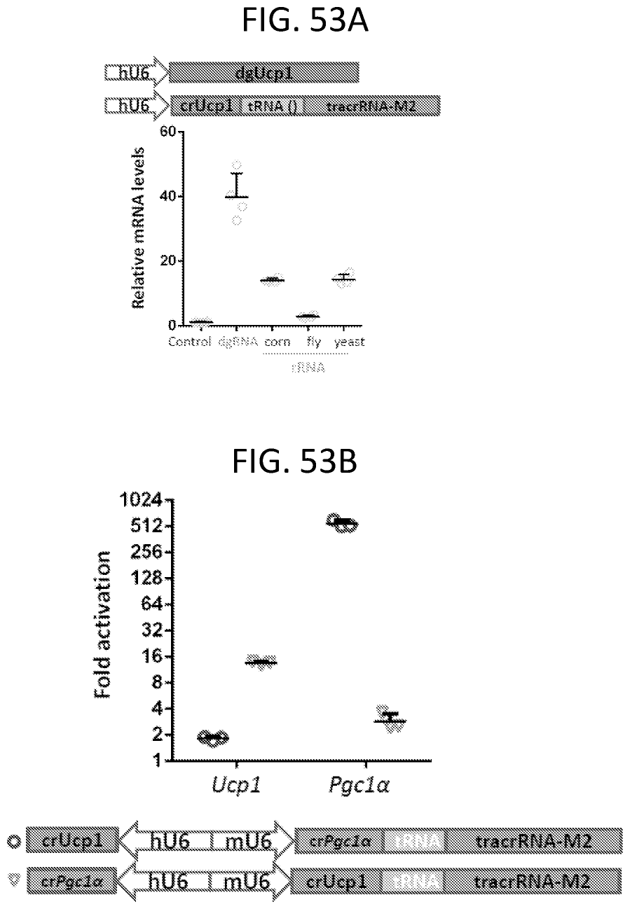

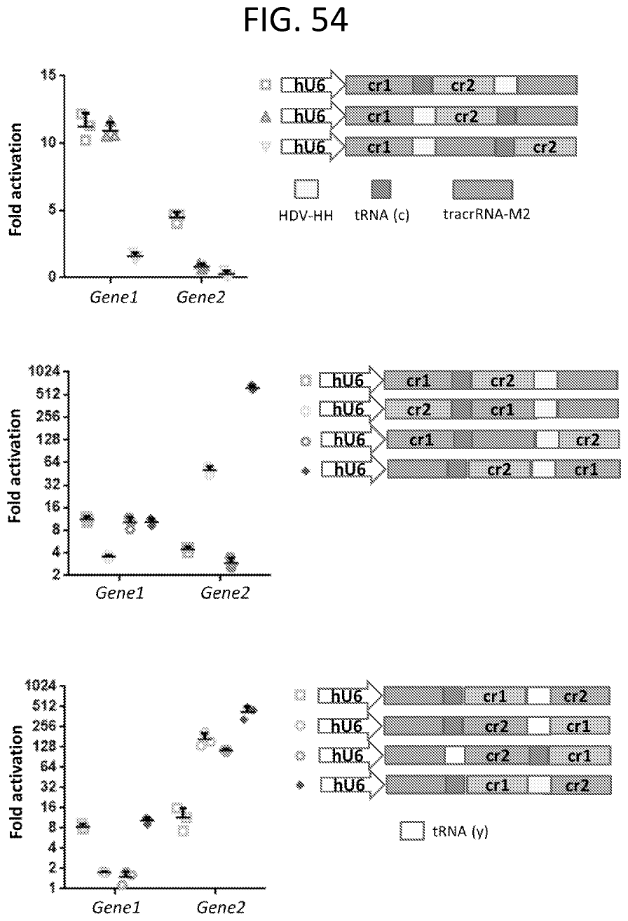

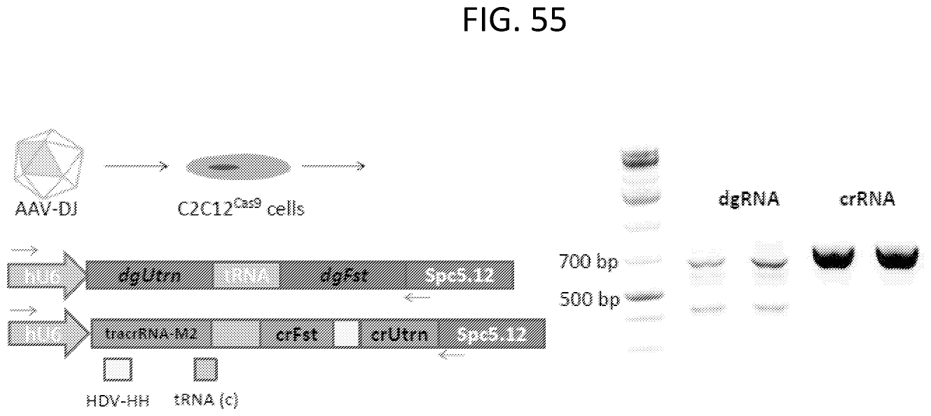

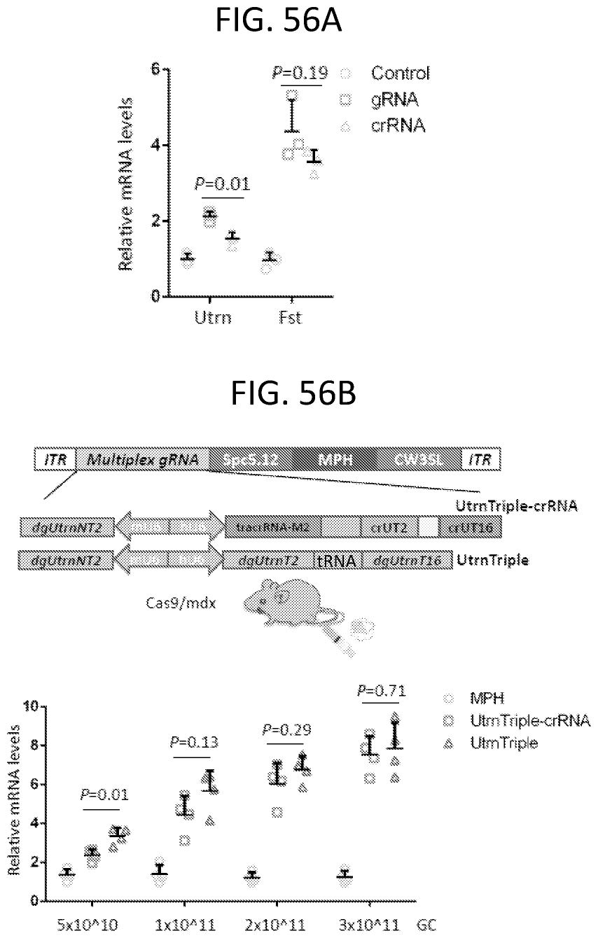

The disclosed mTGA system was further optimized to reduce recombination of the promoter-tRNA construct. Recombination events were monitored by generating a hU6-tRNA construct containing gRNAs with different backbones (FIG. 52). After sequencing the truncated band of the hU6-tRNA construct, it was found that the recombination occurs between the 1^{st} and the 4^{th} MS2 loop (each gRNA contains 2 MS2 loop) to reduce 4 MS2 loops to 2. It was hypothesized that recombination could be minimized if repetitive dgRNA scaffold was reduced. As gRNA can be split into crispr RNA (crRNA) and trans-activating crispr RNA (tracrRNA) elements, a single tracrRNA can be used with multiple crRNAs for multiplexing purposes. To test this, a dgRNA was split into a crispr RNA (crRNA) and a modified trans-activating crispr RNA containing the 2 MS2 loop (tracrRNA-M2), and the polycistronic systems was ligated with a tRNA (FIG. 53A). The crRNA-tRNA-tracrRNA-M2 construct activated the target gene, while its activation efficiency was 2.8-fold lower than dgRNA. Its activation efficiency was also compared using tRNA from different species. tRNAs from yeast and corn were 5-fold more efficient compared to the tRNA from fly (FIG. 53A). Next, it was investigated whether a single tracrRNA-M2 could be used with two crRNAs to activate corresponding targets. Interestingly, when two crRNAs were driven by two different U6 promoters, only the crRNA that shared the same promoter with tracrRNA-M2 had strong activation efficiency (FIG. 53B). Thus, a single promoter to drive a tracrRNA-M2 and two crRNAs (which were separated by different combinations of self-cleaving RNAs) was developed (see, FIG. 54). The activation efficiency of different constructs was tested in vitro using non-viral transfection in N2^{Cas9} cells, and the best construct was determined to be a construct with the tracrRNA-M2 in front of two crRNAs, which were ligated by tRNA and HDV-HH (FIG. 54). The sgRNA following the second tRNA was found to have low activation efficiency in the construct with two tRNAs (FIG. 54). Intriguingly, the recombination found to occur in constructs with one promoter and two sgRNAs separated by a tRNA was eliminated from the construct containing one promoter driving expression of the tracrRNA-M2 and two crRNAs (FIG. 55). However, the activation efficiency of the AAVDJ-hU6-tracrRNA-M2-tRNA-crFst-HDV-HH-crUtrn-MPH was not higher than AAVDJ-hU6-dgUtrnT2-tRNA-dgFst-MPH (FIG. 56A).

### Example 11

### Multiplex crRNA mTGA System in vivo

The in vivo activation of utrophin was compared between UtrnTriple in which two gRNA were driven by the hU6-tRNA construct and UtrnTriple-crRNA in which two gRNAs were driven by the tracrRNA-crRNAs construct (FIG. 56B). Two months after intramuscular injections of different concentrations of AAV9-MPH, AAV9-UtrTriple or AAV9-UtrnTriple-crRNA into TA muscles of Cas9/mdx mice, it was found that AAV9-UtrnTriple had significantly higher activation efficiency than AAV9-UtrnTriple-crRNA at 5×10¹⁰ GC, while the difference was not significant when the AAV concentration was above 1×10¹¹ GC (FIG. 56B). The data indicates that the efficiency of mTGA system is AAV concentration dependent (FIG. 56B).

### Example 12

### Treatment of Liver Disease

This example describes methods that can be used to treat liver fibrosis and/or cirrhosis in vivo. While particular methods are provided, one of skill in the art will recognize that methods that deviate from these specific methods can also be used, including addition or omission of one or more steps.

In this example, crRNAs and/or sgRNAs targeting one or more of HNF1a, HNF4a, FoxA3, and Gata4 are designed for use in the mTGA system described herein. The CMV and/or Col1a2 promoter is used to drive expression of the multiplex crRNAs or sgRNAs. The mTGA constructs are cloned into an AAV vector, such as, AAV9 (herein after referred to as AAV-mTGA).

Mice are injected with AAV-MPH (control) or AAV-mTGA. qPCR and western blot analysis of target genes is used to evaluate activation efficiency. Mouse livers can also be harvested to determine whether fibrosis and/or cirrhosis is reduced following treatment.

In view of the many possible embodiments to which the principles of the disclosure may be applied, it should be recognized that the illustrated embodiments are only examples of the invention and should not be taken as limiting the scope of the invention. Rather, the scope of the invention is defined by the following claims. We therefore claim as our invention all that comes within the scope and spirit of these claims.

1. A nucleic acid encoding multiplex single guide RNAs (sgRNAs) comprising from 5′ to 3′:
a first nucleic acid molecule encoding in reverse orientation a first modified sgRNA operably linked to a first promoter,
a second nucleic acid molecule encoding in forward orientation a second modified sgRNA operably linked to a second promoter,
wherein the encoded first and the second modified sgRNAs comprise at least two modified MS2-binding loops comprising at least two nucleotide changes to the native MS2-binding loop sequence of SEQ ID NO: 16, and wherein the at least two nucleotide changes increase the GC content and/or shorten repetitive content of the modified MS2-binding loop sequence relative to the native MS2-binding loop sequence.

2. The nucleic acid of claim 1, further comprising a third nucleic acid molecule located 3′ of the second nucleic acid molecule, wherein the third nucleic acid encodes in forward orientation a first cleavage site and a third modified sgRNA,
wherein the third modified sgRNA is operably linked to the second promoter and comprises at least two modified MS2-binding loops comprising at least two nucleotide changes to the native MS2-binding loop sequence of SEQ ID NO: 16, and wherein the at least two nucleotide changes increase the GC content and/or shorten repetitive content of the modified MS2-binding loop sequence relative to the native MS2-binding loop sequence.

3. The nucleic acid of claim 1, further comprising a third nucleic acid molecule located 5′ of the first nucleic acid molecule, wherein the third nucleic acid encodes in reverse orientation a first cleavage site and a third modified sgRNA,
wherein the third modified sgRNA is operably linked to the first promoter and comprises at least two modified MS2-binding loops comprising at least two nucleotide changes to the native MS2-binding loop sequence of SEQ ID NO: 16, and wherein the at least two nucleotide changes increase the GC content and/or shorten repetitive content of the modified MS2-binding loop sequence relative to the native MS2-binding loop sequence.

4. The nucleic acid of claim 2, further comprising a fourth nucleic acid molecule located 5′ of the first nucleic acid molecule, wherein the fourth nucleic acid molecule encodes in reverse orientation a second cleavage site and a fourth modified sgRNA.
wherein the fourth modified sgRNA is operably linked to the first promoter and comprises at least two modified MS2-binding loops comprising at least two nucleotide changes to the native MS2-binding loop sequence of SEQ ID NO: 16, and wherein the at least two nucleotide changes increase the GC content and/or shorten repetitive content of the modified MS2-binding loop sequence relative to the native MS2-binding loop sequence.

5. The nucleic acid of claim 4, wherein one or more of the first, second. third, or fourth modified sgRNA are a dgRNA and/or comprise SEQ ID NO: 17, 18, or 19.

6. The nucleic acid of claim 4, wherein the first cleavage site, the second cleavage site, or both, encode a self-cleaving RNA.

7-8. (canceled)

9. The nucleic acid of claim 4, wherein one or more of the first, second, third, or fourth modified sgRNA:
(a) comprise a targeting sequence complementary to a sequence within a promoter region of EEF1a2, Fst, Pdx1, klotho, utrophin, interleukin 10, Six2, OCT4, SOX2, KLF4, c-MYC, MyoD, Mef2b, or Pax7; and/or
(b) comprise a sequence having at least 90% sequence identity to any one of SEQ ID NOS: 10-15 or 42-48, comprise any one of SEQ ID NOS: 10-15 or 42-48, or consist of any one of SEQ ID NOS: 10-15 or 42-48.

10-14. (canceled)

15. The nucleic acid of claim 1, wherein the nucleic acid molecule;
comprises a sequence having at least 90% sequence identity to SEQ ID NO: 3, 4, 5, 6, 53, 54, or 55;
comprises SEQ ID NO: 3, 4, 5, 6, 53, 54, or 55; or
consists of SEQ ID NO: 3, 4, 5, 6, 53, 54, or 55.

16-19. (canceled)

20. A nucleic acid molecule encoding multiplex crisper RNAs (crRNAs) comprising from 5′ to 3′:
a first promoter operably linked to a nucleic acid molecule encoding a modified trans-activating crispr RNA (tracrRNA), a first cleavage site. a first nucleic acid molecule encoding a first crRNA, a second cleavage site, and a second nucleic acid molecule encoding a second crRNA,
wherein the encoded modified tracrRNA comprises at least two modified MS2-binding loops comprising at least two nucleotide changes to the native MS2-binding loop sequence of SEQ ID NO: 16. and wherein the at least two nucleotide changes increase the GC content and/or shorten repetitive content of the modified MS2-binding loop sequence relative to the native MS2-binding loop sequence.

21. The nucleic acid of claim 20, further comprising a second promoter operably linked to a third nucleic acid molecule encoding a third crRNA or a single guide RNA (sgRNA).

22. The nucleic acid of claim 21, wherein;
(a) the second promoter and the third nucleic acid molecule are 3′ of the second nucleic acid molecule encoding a second crRNA: or
(b) the second promoter and the third nucleic acid molecule are in reverse orientation and located S′ of the first promoter.

23. The nucleic acid of claim 20, wherein the first or second cleavage site encode a pre-transfer RNA (pre-tRNA) or a self-cleaving ribozyme.

24. (canceled)

25. The nucleic acid of claim 20, wherein the modified tracrRNA:
comprises a sequence having at least 90% sequence identity to SEQ ID NO: 7;
comprises SEQ ID NO: 7; or
consists of SEQ ID NO: 7.

26. The nucleic acid of claim 21, wherein one or more of the first crRNA, the second crRNA, the third crRNA, or the sgRNA:
(i) comprise a targeting sequence complementary to a sequence within a promoter region of EEF1a2, Fst, Pdx1, klotho, utrophin, interleukin 10, Six2, OCT4, SOX2, KLF4, c-MYC, MyoD. Mef2b, or Pax7; and/or
(ii) comprises a sequence having at least 90% sequence identity to SEQ ID NO: 8, 9, 10, 11, 12, 13, 14, 15, 42, 43, 44, 45, 46, 47, 48, 49, 50, 51, or 52; comprises SEQ ID NO: 8, 9, 10, 11, 12, 13, 14, 15, 42, 43, 44, 45, 46, 47 48, 49, 50, 51, or 52; or consists of SEQ ID NO: 8, 9, 10, 11, 12, 13, 14, 15, 42, 43, 44, 45, 46, 47, 48, 49, 50, 51, or 52.

27-28. (canceled)

29. The nucleic acid of claim 20, wherein the nucleic acid molecule:
comprises a sequence having at least 90% sequence identity to SEQ ID NO: 1 or 2;
comprises SEQ ID NO: 1 or 2; or
consists of SEQ ID NO: 1 or 2.

30. (canceled)

31. The nucleic acid of claim 21, wherein the sgRNA is a dead guide RNA (dgRNA).

32. An RNA molecule encoded by the nucleic acid molecule of claim 1.

33. A viral vector comprising the nucleic acid of claim 1.

34. A composition, comprising:
the nucleic acid molecule of claim 1, or a viral vector including the nucleic acid molecule of claim 1, and
a pharmaceutically acceptable carrier.

35. A kit, comprising the nucleic acid molecule of claim 1 or a viral vector including the nucleic acid molecule of claim 1, and
a nucleic acid encoding a Cas9 protein or dead Cas9 (dCas9) protein, and/or
a nucleic acid encoding an MS2-transcriptional activator fusion protein.

36. A multiplex targeted gene activation (mTGA) system, comprising:
(a) a first vector comprising a nucleic acid encoding a Cas9 or dCas9; and
(b) a second vector comprising the nucleic acid of claim 1, and a nucleic acid encoding an MS2-transcriptional activator fusion protein.

37. A method of increasing expression of at least one gene product in a subject, comprising:
administering a therapeutically effective amount of the multiplex targeted gene activation (mTGA) system of claim 36 to the subject,
thereby increasing expression of the at least one gene product in the subject.

38. The method of claim 37, wherein the method comprises treating a disease associated with reduced or no expression of a gene, optionally wherein the disease is type I diabetes, Duchenne muscular dystrophy, liver disease, or acute kidney disease.

39-42. (canceled)


SEQUENCE LISTING (61 sequences)

SEQ ID NO: 1 - DNA, 444 bp, Artificial Sequence
  Exemplary DNA sequence encoding multiplex crRNAs
gaaccattcaaaacagcatagcaagttaaaataaggctagtccgttatcaacttgaaaaagtggcaccgagtcggtgcgggagcggccagcatgaggatcacccatgcctgcagggccgccacgagcggggccaacatgaggatcacccatgtctgcagggccccgctcgtgttcccaacaaagcaccagtggtctagtggtagaatagtaccctgccacggtacagacccgggttcgattcccggctggtgcagagagcagcagttggttttagagctatgctgttttgggccggcatggtcccagcctcctcgctggcgccggctgggcaacatgcttcggcatggcgaatgggacattcaactgatgagtccgtgaggacgaaacgagtaagctcgtcttgaataaagggcagttttagagctatgctgttttgttttttt

SEQ ID NO: 2 - DNA, 1243 bp, Artificial Sequence
  Exemplary DNA sequence encoding multiplex crRNAs with a dgRNA
aaaaaaagcaccagccgggaatcgaacccgggtctgtaccgtggcagggtactattctaccactagaccactggtgctttgttgcaccgactcggtgccacttggccctgcaggcatgggtgatcctcatgctggccaagttgataacggactagccttatttcaacttgctaggccctgcaggcatgggtgatcctcatgctggcctagctctgaaacgtcgtgcgtgctggcaaacaaggcttttctccaagggatatttatagtctcaaaacacacaattactttacagttagggtgagtttccttttgtgctgttttttaaaataataatttagtatttgtatctcttatagaaatccaagcctatcatgtaaaatgtagctagtattaaaaagaacagattatctgtcttttatcgcacattaagcctctatagttactaggaaatattatatgcaaattaaccggggcaggggagtagccgagcttctcccacaagtctgtgcgagggggccggcgcgggcctagagatggcggcgtcggatcgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggaaccattcaaaacagcatagcaagttaaaataaggctagtccgttatcaacttgaaaaagtggcaccgagtcggtgcgggagcggccagcatgaggatcacccatgcctgcagggccgccacgagcggggccaacatgaggatcacccatgtctgcagggccccgctcgtgttcccaacaaagcaccagtggtctagtggtagaatagtaccctgccacggtacagacccgggttcgattcccggctggtgcagagagcagcagttggttttagagctatgctgttttgggccggcatggtcccagcctcctcgctggcgccggctgggcaacatgcttcggcatggcgaatgggacattcaactgatgagtccgtgaggacgaaacgagtaagctcgtcttgaataaagggcagttttagagctatgctgttttgttttttt

SEQ ID NO: 3 - DNA, 956 bp, Artificial Sequence
  Exemplary DNA sequence encoding multiplex sgRNAs
aaaaaaagcaccagccgggaatcgaacccgggtctgtaccgtggcagggtactattctaccactagaccactggtgctttgttgcaccgactcggtgccacttggccctgcaggcatgggtgatcctcatgctggccaagttgataacggactagccttatttcaacttgctaggccctgcaggcatgggtgatcctcatgctggcctagctctgaaacgtcgtgcgtgctggcaaacaaggcttttctccaagggatatttatagtctcaaaacacacaattactttacagttagggtgagtttccttttgtgctgttttttaaaataataatttagtatttgtatctcttatagaaatccaagcctatcatgtaaaatgtagctagtattaaaaagaacagattatctgtcttttatcgcacattaagcctctatagttactaggaaatattatatgcaaattaaccggggcaggggagtagccgagcttctcccacaagtctgtgcgagggggccggcgcgggcctagagatggcggcgtcggatcgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcaaagcggcaggaggtttcagagctaggccagcatgaggatcacccatgcctgcagggcctagcaagttgaaataaggctagtccgttatcaacttggccagcatgaggatcacccatgcctgcagggccaagtggcaccgagtcggtgcttttttt

SEQ ID NO: 4 - DNA, 1195 bp, Artificial Sequence
  Exemplary DNA sequence encoding multiplexed sgRNAs
aaaaaaagcaccagccgggaatcgaacccgggtctgtaccgtggcagggtactattctaccactagaccactggtgctttgttgcaccgactcggtgccacttggccctgcaggcatgggtgatcctcatgctggccaagttgataacggactagccttatttcaacttgctaggccctgcaggcatgggtgatcctcatgctggcctagctctgaaacgtcgtgcgtgctggcaaacaaggcttttctccaagggatatttatagtctcaaaacacacaattactttacagttagggtgagtttccttttgtgctgttttttaaaataataatttagtatttgtatctcttatagaaatccaagcctatcatgtaaaatgtagctagtattaaaaagaacagattatctgtcttttatcgcacattaagcctctatagttactaggaaatattatatgcaaattaaccggggcaggggagtagccgagcttctcccacaagtctgtgcgagggggccggcgcgggcctagagatggcggcgtcggatcgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggacaatttgaataaagggcagtttcagagctaggccagcatgaggatcacccatgcctgcagggcctagcaagttgaaataaggctagtccgttatcaacttggccagcatgaggatcacccatgcctgcagggccaagtggcaccgagtcggtgcaacaaagcgcaagtggtttagtggtaaaatccaacgttgccatcgttgggcccccggttcgattccgggcttgcgcaaaggtagagagcagcagttggtttcagagctaggccagcatgaggatcacccatgcctgcagggcctagcaagttgaaataaggctagtccgttatcaacttggccagcatgaggatcacccatgcctgcagggccaagtggcaccgagtcggtgcttttttt

SEQ ID NO: 5 - DNA, 1183 bp, Artificial Sequence
  Exemplary DNA sequence encoding multiplex sgRNAs
aaaaaaagcaccagccgggaatcgaacccgggtctgtaccgtggcagggtactattctaccactagaccactggtgctttgttgcaccgactcggtgccacttggccctgcaggcatgggtgatcctcatgctggccaagttgataacggactagccttatttcaacttgctaggccctgcaggcatgggtgatcctcatgctggcctagctctgaaacgtcgtgcgtgctggcaaacaaggcttttctccaagggatatttatagtctcaaaacacacaattactttacagttagggtgagtttccttttgtgctgttttttaaaataataatttagtatttgtatctcttatagaaatccaagcctatcatgtaaaatgtagctagtattaaaaagaacagattatctgtcttttatcgcacattaagcctctatagttactaggaaatattatatgcaaattaaccggggcaggggagtagccgagcttctcccacaagtctgtgcgagggggccggcgcgggcctagagatggcggcgtcggatcgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgcccctcctttccgtttcagagctaggccagcatgaggatcacccatgcctgcagggcctagcaagttgaaataaggctagtccgttatcaacttggccagcatgaggatcacccatgcctgcagggccaagtggcaccgagtcggtgcaacaaagcgcaagtggtttagtggtaaaatccaacgttgccatcgttgggcccccggttcgattccgggcttgcgcacaaagcggcaggaggtttcagagctaggccagcatgaggatcacccatgcctgcagggcctagcaagttgaaataaggctagtccgttatcaacttggccagcatgaggatcacccatgcctgcagggccaagtggcaccgagtcggtgcttttttt

SEQ ID NO: 6 - DNA, 1334 bp, Artificial Sequence
  Exemplary DNA sequence encoding multiplex sgRNAs
aaaaaaagcaccgactcggtgccacttggccctgcaggcatgggtgatcctcatgctggccaagttgataacggactagccttatttcaacttgctaggccctgcaggcatgggtgatcctcatgctggcctagctctgaaactgccctttattcaatgcaccagccgggaatcgaacccgggtctgtaccgtggcagggtactattctaccactagaccactggtgctttgttgcaccgactcggtgccacttggccctgcaggcatgggtgatcctcatgctggccaagttgataacggactagccttatttcaacttgctaggccctgcaggcatgggtgatcctcatgctggcctagctctgaaacgtcgtgcgtgctggcaaacaaggcttttctccaagggatatttatagtctcaaaacacacaattactttacagttagggtgagtttccttttgtgctgttttttaaaataataatttagtatttgtatctcttatagaaatccaagcctatcatgtaaaatgtagctagtattaaaaagaacagattatctgtcttttatcgcacattaagcctctatagttactaggaaatattatatgcaaattaaccggggcaggggagtagccgagcttctcccacaagtctgtgcgagggggccggcgcgggcctagagatggcggcgtcggatcgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgcccctcctttccgtttcagagctaggccagcatgaggatcacccatgcctgcagggcctagcaagttgaaataaggctagtccgttatcaacttggccagcatgaggatcacccatgcctgcagggccaagtggcaccgagtcggtgcaacaaagcgcaagtggtttagtggtaaaatccaacgttgccatcgttgggcccccggttcgattccgggcttgcgcacaaagcggcaggaggtttcagagctaggccagcatgaggatcacccatgcctgcagggcctagcaagttgaaataaggctagtccgttatcaacttggccagcatgaggatcacccatgcctgcagggccaagtggcaccgagtcggtgcttttttt

SEQ ID NO: 7 - DNA, 178 bp, Artificial Sequence
  Exemplary DNA sequence encoding a modified tracrRNA
ggaaccattcaaaacagcatagcaagttaaaataaggctagtccgttatcaacttgaaaaagtggcaccgagtcggtgcgggagcggccagcatgaggatcacccatgcctgcagggccgccacgagcggggccaacatgaggatcacccatgtctgcagggccccgctcgtgttccc

SEQ ID NO: 8 - DNA, 43 bp, Artificial Sequence
  Exemplary DNA sequence encoding crUT2
ttgaataaagggcagttttagagctatgctgttttgttttttt

SEQ ID NO: 9 - DNA, 43 bp, Artificial Sequence
  Exemplary DNA sequence encoding crUT16
gagagcagcagttggttttagagctatgctgttttgttttttt

SEQ ID NO: 10 - DNA, 157 bp, Artificial Sequence
  Exemplary DNA sequence encoding dgFST
caaagcggcaggaggtttcagagctaggccagcatgaggatcacccatgcctgcagggcctagcaagttgaaataaggctagtccgttatcaacttggccagcatgaggatcacccatgcctgcagggccaagtggcaccgagtcggtgcttttttt

SEQ ID NO: 11 - DNA, 157 bp, Artificial Sequence
  Exemplary DNA sequence encoding dgEef1a2
tgcccctcctttccgtttcagagctaggccagcatgaggatcacccatgcctgcagggcctagcaagttgaaataaggctagtccgttatcaacttggccagcatgaggatcacccatgcctgcagggccaagtggcaccgagtcggtgcttttttt

SEQ ID NO: 12 - DNA, 157 bp, Artificial Sequence
  Exemplary DNA sequence encoding dgUtrnNT2
ccagcacgcacgacgtttcagagctaggccagcatgaggatcacccatgcctgcagggcctagcaagttgaaataaggctagtccgttatcaacttggccagcatgaggatcacccatgcctgcagggccaagtggcaccgagtcggtgcttttttt

SEQ ID NO: 13 - DNA, 157 bp, Artificial Sequence
  Exemplary DNA sequence encoding dgUtrn
ttgaataaagggcagtttcagagctaggccagcatgaggatcacccatgcctgcagggcctagcaagttgaaataaggctagtccgttatcaacttggccagcatgaggatcacccatgcctgcagggccaagtggcaccgagtcggtgcttttttt

SEQ ID NO: 14 - DNA, 157 bp, Artificial Sequence; identical to SEQ ID NO: 13
  Exemplary DNA sequence encoding dgUtrnT2

SEQ ID NO: 15 - DNA, 157 bp, Artificial Sequence
  Exemplary DNA sequence encoding dgUtrnT16
gagagcagcagttggtttcagagctaggccagcatgaggatcacccatgcctgcagggcctagcaagttgaaataaggctagtccgttatcaacttggccagcatgaggatcacccatgcctgcagggccaagtggcaccgagtcggtgcttttttt

SEQ ID NO: 16 - DNA, 34 bp, Artificial Sequence
  Exemplary DNA sequence encoding a native MS2-binding loop
ggccaacatgaggatcacccatgtctgcagggcc

SEQ ID NO: 17 - DNA, 36 bp, Artificial Sequence
  Exemplary DNA sequence encoding a modified MS2-binding loop
tgctgaacatgaggatcacccatgtctgcagcagca

SEQ ID NO: 18 - DNA, 36 bp, Artificial Sequence
  Exemplary DNA sequence encoding a modified MS2-binding loop
gggccaacatgaggatcacccatgtctgcagggccc

SEQ ID NO: 19 - DNA, 34 bp, Artificial Sequence
  Exemplary DNA sequence encoding a modified MS2-binding loop
ggccagcatgaggatcacccatgcctgcagggcc

SEQ ID NO: 20 - DNA, 83 bp, Saccharomyces cerevisiae
aacaaagcgcaagtggtttagtggtaaaatccaacgttgccatcgttgggcccccggttcgattccgggcttgcgcacgaaat

SEQ ID NO: 21 - DNA, 77 bp, Zea mays
aacaaagcaccagtggtctagtggtagaatagtaccctgccacggtacagacccgggttcgattcccggctggtgca

SEQ ID NO: 22 - DNA, 111 bp, Artificial Sequence
  Exemplary DNA sequence encoding a hammerhead RNA
ggccggcatggtcccagcctcctcgctggcgccggctgggcaacatgcttcggcatggcgaatgggactgctggctgatgagtccgtgaggacgaaacgagtaagctcgtc

SEQ ID NO: 23 - DNA, 901 bp, Homo sapiens
ggcccggtctttggcttggcatcctgaccccatatgagcatcagctacaaggcgctgaggtgcagcggggtggggcgctgggcgggggggcctgggtctgtctggatctgactcgcccttggctggcgctgtttcccagcagcagccggaggtcggcgcacccggaggggagggtccctggaagatgtcagtgggtctgggagcgggcttccggcgttccctgcaccgtgggagaccagcctctcagggggagggtggttctgcgctggatcctcggggcctgtcatggtgcgcccaggagggcaggcacgtgaggacagggactggaaaccagcagatttccaccctgaggcctgcacccccgggcctcattagggagagcccctcagagccgggcttcgttggttctggggcgtcccccatgagcagggccggggaggggccggtagacccaggctcgtctcccaggctgcagcccacctgctcccctcccccgcctgccggctccggtcctcggcgtctgccctgtccccggggaccgcttttcgcggctcaagcgtgttcctgccctgagccggctctcgccccgtctcccgggcccgccgcgctctccccgcgccgtctccgtcccggtccctccctcccgccgcctccctgccctgccccccgccccgcccccgcccgcggcgcgtttctcccccgcctcccgcgtccgtctttgcagcccgcgcctcccgcatcgcctcgcgtccccgtggcgcccgcccgcgcgcgtccgcgccccgccccctcccgcgcggttccgcattggcgtgctgcagggcgcggtgcactgcgccgccaccgtcaataggtggaccccctcccggagataaaaccgccggcgccggcgccgccagtc

SEQ ID NO: 24 - DNA, 721 bp, Homo sapiens
ggagccgaggagactgagagacagacagaggcacacaggacagaaactggggagtctccaggcgggagaggaagggggggccagaccgcctacgtcggcgcccccgctccgggctccgactccagacgccgcgaagtgaaaggggagaaaagaaagggagagggcgaggctgtgccgcggggagaccgggcctgaggtgttaaacatttttgtttgcttccgactagtccagacgaagggccgcgtctcggtagcgctctgccagggtggaaggtgccggggccggggttcctagcaacacctctgggctgggggtggctgcaaagtcaggcactcacagacccagacacaaaacctcgcgggtcccgcgcccaggctgcgggtgcccggaaccgccgcgaggccggcgcgctccgacccgacccggggcgggatatttgggcagcccggggctcttcggccgtttgcaaaagtctctttggagcggaggagaggcagcacggagacaaactcccgggttccccccgccaccgcctccagcgcccccaccgcgccctccctctcacactcgcgcgcgcgcgcacacacactcacacacacactcacacacacacccgccaccccgggcgcgccggcgctgccggcgagcggcggcgagcaggacttgaagtgggtgttcttccccactccccacccccgacgcgtagcccccaacccccgc

SEQ ID NO: 25 - DNA, 633 bp, Homo sapiens
ttaaaaaaaagaatttaaaaaagtctctgtgaatgcttcagaagttaccgtttacaccccagaagtacttgcagcacatccacaagtaaaaacacacaacgaatgccagagtttcgtgtgttttttaaccgacatctttgtggctgtgaacaaacttcataaataaaatagaatcaaatgcttctgacctagagagctgggtctgcaaactttttttttatcgtattccgcaacagttaaataaaaaattaaaaactcaacatgtctccttgtaaactacatcaattaacaaacacactatgtccattatcaaatataatagaaaaaatataggaaaatagaaaatagaaaaatataggaaaatagaaacttttaagccacggtgaaaatgtttctataaatgagtggttctaatgttttcgtgagcgcccattttggggagcaccgccagctgcccgttcaggagtgtgcagcaaactcagctgagagagaaaattggaacaaaagcaggtgctcgcgggtacctgggcctagcctcttagtgcggccagccaggccaatcacggcccccggctgaaccacgtggggccccgcggagcctatggtgcggcggccggcccgccggtccgcgct

SEQ ID NO: 26 - DNA, 721 bp, Homo sapiens
gtggctctgcaacttctgtcaaaagggctctttggcaacaggaaaaacgtcatggctccattgtattgtagaggatgggaatgggtgttccggctaaattctccctcccctttccctccacagctcagatggcaaatgtgcgacccagggacctcccgctccagcagacctgtgcgcacaactttgcacagattacctgctaagtcagagccgaaaggtaacacagatgccaaaggataataaaggtgaatgagatttactcaaaattggaaacttggtgtttggtttttcaggagaacaatcaacgactgtgatttgaagttcaccagggtattctgagagatctaatcaaagatagagtgctggtttgaaattattaaaaggtaacagtaaaagggagagcaaaaccccagtcccaacgcaacccataaatctactttgtcttcctcgaaagaggggcgcgggtgggcgcgtctccccgcgagcatctcacctaagggggaatccctttcagcgcacggcgaagttccccctcggctgtcccacctggcagtccctctaggatttcggccagtccctaattggctccagcaatgtccagccggagcttctttgggcctccgagtgggagaaaagtgagagcaggtgcttccccagcggcgcgctccgctagggcccggcaggatcccgcccccaagtcggggaaagttggtcggcgcct

SEQ ID NO: 27 - DNA, 361 bp, Homo sapiens
aactaggggtaaaaaaaaaatcagcaacgtcagcaaactgagatggggtgagttggaaggcagattggaatttatctcttaaaaaaatatcaccctaactagagacctgttttgcctaaggggacgtgactcacattttcggataatctgaataaggggaattgtgtctgctcgaggcatccattctggttcggtctccggactcccggctcccggcacgcacggttcactctggagcgcgcgccccaggccagccaagcgccgagccgggctgctgcgggctgggagggcgcgcagggccggcgctgattgacggggcgcgcagtcaggtgacttggggcgccaagttcccgacgcggtg

SEQ ID NO: 28 - DNA, 721 bp, Homo sapiens
taagaagctttcagcaagtgcagactactcttacccacttcccccaagcacagttggggtgggggacagctgaagaggtggaaacatgtgcctgagaatcctaatgaaatcggggtaaaggagcctggaacacatcctgtgaccccgcctgtactgtaggaagccagtctctggaaagtaaaatggaagggctgcttgggaactttgaggatatttagcccaccccctcatttttacttggggaaactaaggcccagagacctaaggtgactgcctaagttagcaaggagaagtcttgggtattcatcccaggttggggggacccaattatttctcaatcccattgtattctggaatgggcaatttgtccacgtcactgtgacctaggaacacgcgaatgagaacccacagctgagggcctctgcgcacagaacagctgttctccccaggaaatcaactttttttaattgagaagctaaaaaattattctaagagaggtagcccatcctaaaaatagctgtaatgcagaagttcatgttcaaccaatcatttttgcttacgatgcaaaaattgaaaactaagtttattagagaggttagagaaggaggagctctaagcagaaaaaatcctgtgccgggaaaccttgattgtggctttttaatgaatgaagaggcctccctgagcttacaatataaaagggggacagagaggtgaaggtcta

SEQ ID NO: 29 - DNA, 861 bp, Homo sapiens
gtcgcccctctcccccgccccggtgggcagactgcgggtctgcgccgtccggggttctgcgtcgcagctgccggccggagtcagcttccatagaggccacacggaactgcctggcgctcctcgggctgtgggacccgtggggttaagtctgagtccccgcccggcgaggagcagagagcgcagagttggggcggtacaggccgccaggcagccggcggggctaggagagggaggaaaggcgggatcctccgggaagtcgattctccggcgtccgcctgcggccactgccaaatcttccccatttctttcgtctactccctccccttttccctcgaggaccgctgagtccagagtttctaggatgggggtggggcgctgtcagcagaaaaagccaagtctttgggcggcacccgagcacgtccaaactctcccatcccactggcctgcgccggggtagaatgtgcccggtgaacagagagcctgggagggacgcggtgacctggggagaaggggaaccctgtagggtctgggcgaggctgcagagccctctcctagccaaagctgcccaaactttcttcccctggagtctccttccacccctctccctccccttcctcctggacacccccttaaacggtctccgccttcccttctctcctcttctctccccacctcgatccaccccttttcgtcttcgcccgctccccccgctctcctgtcctcctcctccctccctctttgggcatccgccccgtcaatctccgccgccgccggccccaacccggcccctctccgcctcccaggctctcagagcgccccaggctccagtagagccgccctcagttctgcgcggagcggggc

SEQ ID NO: 30 - DNA, 4101 bp, Artificial Sequence
  Exemplary DNA sequence encoding Cas9
gacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 31 - protein, 1368 aa, Artificial Sequence
  Exemplary Cas9 amino acid sequence
MDKKYSIGLDIGTNSVGWAVITDDYKVPSKKFKVLGNTDRHSIKKNLIGALLFDSGEIAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRLNSEITKAPLSASMIKRYDEHHQDLTLLKALVRQQLPEKYKEIFFDQSKNGYAGYIDGGASQEEFYKFIKPILEKMDGTEELLAKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFIKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMLAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVRKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPTAFKYFDTTIDRKRYTSTKEVLDATFIHQSITGLYETRIDLSQLGGD

SEQ ID NO: 32 - DNA, 4101 bp, Artificial Sequence
  Exemplary DNA sequence encoding dCas9
gacaagaagtactccattgggctcgctatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggctgctatcgtgccccagtcttttctcaaagatgattctattgataataaagtgttgacaagatccgataaagctagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagac

SEQ ID NO: 33 - protein, 1367 aa, Artificial Sequence
  Exemplary dCas9 amino acid sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGG

SEQ ID NO: 34 - DNA, 1419 bp, Artificial Sequence
  Exemplary DNA sequence encoding a MS2-transcriptional activator fusion protein
gcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgctggaggaggtggaagcggaggaggaggaagcggaggaggaggtagcggacctaagaaaaagaggaaggtggcggccgctggatccccttcagggcagatcagcaaccaggccctggctctggcccctagctccgctccagtgctggcccagactatggtgccctctagtgctatggtgcctctggcccagccacctgctccagcccctgtgctgaccccaggaccaccccagtcactgagcgctccagtgcccaagtctacacaggccggcgaggggactctgagtgaagctctgctgcacctgcagttcgacgctgatgaggacctgggagctctgctggggaacagcaccgatcccggagtgttcacagatctggcctccgtggacaactctgagtttcagcagctgctgaatcagggcgtgtccatgtctcatagtacagccgaaccaatgctgatggagtaccccgaagccattacccggctggtgaccggcagccagcggccccccgaccccgctccaactcccctgggaaccagcggcctgcctaatgggctgtccggagatgaagacttctcaagcatcgctgatatggactttagtgccctgctgtcacagatttcctctagtgggcagggaggaggtggaagcggcttcagcgtggacaccagtgccctgctggacctgttcagcccctcggtgaccgtgcccgacatgagcctgcctgaccttgacagcagcctggccagtatccaagagctcctgtctccccaggagccccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctcctga

SEQ ID NO: 35 - protein, 473 aa, Artificial Sequence
  Exemplary MS2-p65-HSF1 amino acid sequence
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSAGGGGSGGGGSGGGGSGPKKKRKVAAAGSPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 36 - DNA, 276 bp, Artificial Sequence
  Exemplary DNA sequence encoding a 7SK promoter
tttaattctagtactatgcatcgtctcattgtctgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagcaacttgacctaagtgtaaagttgagacttccttcaggtttatatagcttgtgcgccgcttgggtacctcg

SEQ ID NO: 37 - DNA, 361 bp, Artificial Sequence
  Exemplary DNA sequence encoding a Spc5.12 promoter
caccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatccccc

SEQ ID NO: 38 - DNA, 410 bp, Artificial Sequence
  Exemplary DNA sequence encoding a Col1a2 promoter
agatctgtaaagagcccacgtaggtgtcctaaagtgcttccaaacttggcaagggcgagagagggcgggtggctggggagggcggaggtatgcagacagggagtcagagttccccctcgaaagcctcaaaagtgtccacgtcctcaaaaagaatggaaccaatttaagaagccccgtagccacgtccctcccccctcggctccctcccctgctcccccgcagtctcctcccagcactgagtcccgggcccctagccctagccctcccattggtggagacgtttttggaggcaccctccggctggggaaacttttcccatataaataaggcaggtctgggctttattattttagcaccacggcagcaggaggtttcgactaagttggagggaacggtccacgattgcatgc

SEQ ID NO: 39 - DNA, 316 bp, Artificial Sequence
  Exemplary DNA sequence encoding an mU6 promoter
gatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttg

SEQ ID NO: 40 - DNA, 250 bp, Artificial Sequence
  Exemplary DNA sequence encoding an hU6 promoter
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccg

SEQ ID NO: 41 - DNA, 224 bp, Artificial Sequence
  Exemplary DNA sequence encoding an H1 promoter
gaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccactctttccca

SEQ ID NO: 42 - DNA, 157 bp, Artificial Sequence
  Exemplary DNA sequence encoding dgMyoD
agagttggtagagtgtttcagagctaggccagcatgaggatcacccatgcctgcagggcctagcaagttgaaataaggctagtccgttatcaacttggccagcatgaggatcacccatgcctgcagggccaagtggcaccgagtcggtgcttttttt

SEQ ID NO: 43 - DNA, 157 bp, Artificial Sequence
  Exemplary DNA sequence encoding dgMef2b
actgagcatagctcgtttcagagctaggccagcatgaggatcacccatgcctgcagggcctagcaagttgaaataaggctagtccgttatcaacttggccagcatgaggatcacccatgcctgcagggccaagtggcaccgagtcggtgcttttttt

SEQ ID NO: 44 - DNA, 157 bp, Artificial Sequence
  Exemplary DNA sequence encoding dgPax7
acaccggctgccgtgtttcagagctaggccagcatgaggatcacccatgcctgcagggcctagcaagttgaaataaggctagtccgttatcaacttggccagcatgaggatcacccatgcctgcagggccaagtggcaccgagtcggtgcttttttt

SEQ ID NO: 45 - DNA, 157 bp, Artificial Sequence
  Exemplary DNA sequence encoding dgOCT4
ggggacctgcactggtttcagagctaggccagcatgaggatcacccatgcctgcagggcctagcaagttgaaataaggctagtccgttatcaacttggccagcatgaggatcacccatgcctgcagggccaagtggcaccgagtcggtgcttttttt

SEQ ID NO: 46 - DNA, 157 bp, Artificial Sequence
  Exemplary DNA sequence encoding dgSOX2
ccggcagcgaggctgtttcagagctaggccagcatgaggatcacccatgcctgcagggcctagcaagttgaaataaggctagtccgttatcaacttggccagcatgaggatcacccatgcctgcagggccaagtggcaccgagtcggtgcttttttt

SEQ ID NO: 47 - DNA, 157 bp, Artificial Sequence
  Exemplary DNA sequence encoding dgKLF
atagcaacgatggagtttcagagctaggccagcatgaggatcacccatgcctgcagggcctagcaagttgaaataaggctagtccgttatcaacttggccagcatgaggatcacccatgcctgcagggccaagtggcaccgagtcggtgcttttttt

SEQ ID NO: 48 - DNA, 157 bp, Artificial Sequence
  Exemplary DNA sequence encoding dgMYC
caaagcagagggcggtttcagagctaggccagcatgaggatcacccatgcctgcagggcctagcaagttgaaataaggctagtccgttatcaacttggccagcatgaggatcacccatgcctgcagggccaagtggcaccgagtcggtgcttttttt

SEQ ID NO: 49 - DNA, 43 bp, Artificial Sequence
  Exemplary DNA sequence encoding crUCP1
gagtgacgcgcggcgttttagagctatgctgttttgttttttt

SEQ ID NO: 50 - DNA, 43 bp, Artificial Sequence
  Exemplary DNA sequence encoding crPgc1a
gcgttacttcactggttttagagctatgctgttttgttttttt

SEQ ID NO: 51 - DNA, 43 bp, Artificial Sequence
  Exemplary DNA sequence encoding crFST
caaagcggcaggaggttttagagctatgctgttttgttttttt

SEQ ID NO: 52 - DNA, 43 bp, Artificial Sequence; identical to SEQ ID NO: 8
  Exemplary DNA sequence encoding crUtrn

SEQ ID NO: 53 - DNA, 956 bp, Artificial Sequence
  Exemplary DNA sequence encoding multiplex sgRNAs
aaaaaaagcaccagccgggaatcgaacccgggtctgtaccgtggcagggtactattctaccactagaccactggtgctttgttgcaccgactcggtgccacttggccctgcaggcatgggtgatcctcatgctggccaagttgataacggactagccttatttcaacttgctaggccctgcaggcatgggtgatcctcatgctggcctagctctgaaacgtcgtgcgtgctggcaaacaaggcttttctccaagggatatttatagtctcaaaacacacaattactttacagttagggtgagtttccttttgtgctgttttttaaaataataatttagtatttgtatctcttatagaaatccaagcctatcatgtaaaatgtagctagtattaaaaagaacagattatctgtcttttatcgcacattaagcctctatagttactaggaaatattatatgcaaattaaccggggcaggggagtagccgagcttctcccacaagtctgtgcgagggggccggcgcgggcctagagatggcggcgtcggatcgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgttgaataaagggcagtttcagagctaggccagcatgaggatcacccatgcctgcagggcctagcaagttgaaataaggctagtccgttatcaacttggccagcatgaggatcacccatgcctgcagggccaagtggcaccgagtcggtgcttttttt

SEQ ID NO: 54 - DNA, 956 bp, Artificial Sequence
  Exemplary DNA sequence encoding multiplex sgRNAs
aaaaaaagcaccagccgggaatcgaacccgggtctgtaccgtggcagggtactattctaccactagaccactggtgctttgttgcaccgactcggtgccacttggccctgcaggcatgggtgatcctcatgctggccaagttgataacggactagccttatttcaacttgctaggccctgcaggcatgggtgatcctcatgctggcctagctctgaaacgtcgtgcgtgctggcaaacaaggcttttctccaagggatatttatagtctcaaaacacacaattactttacagttagggtgagtttccttttgtgctgttttttaaaataataatttagtatttgtatctcttatagaaatccaagcctatcatgtaaaatgtagctagtattaaaaagaacagattatctgtcttttatcgcacattaagcctctatagttactaggaaatattatatgcaaattaaccggggcaggggagtagccgagcttctcccacaagtctgtgcgagggggccggcgcgggcctagagatggcggcgtcggatcgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgcccctcctttccgtttcagagctaggccagcatgaggatcacccatgcctgcagggcctagcaagttgaaataaggctagtccgttatcaacttggccagcatgaggatcacccatgcctgcagggccaagtggcaccgagtcggtgcttttttt

SEQ ID NO: 55 - DNA, 1107 bp, Artificial Sequence
  Exemplary DNA sequence encoding multiplex sgRNAs
aaaaaaagcaccgactcggtgccacttggccctgcaggcatgggtgatcctcatgctggccaagttgataacggactagccttatttcaacttgctaggccctgcaggcatgggtgatcctcatgctggcctagctctgaaactgccctttattcaatgcaccagccgggaatcgaacccgggtctgtaccgtggcagggtactattctaccactagaccactggtgctttgttgcaccgactcggtgccacttggccctgcaggcatgggtgatcctcatgctggccaagttgataacggactagccttatttcaacttgctaggccctgcaggcatgggtgatcctcatgctggcctagctctgaaacgtcgtgcgtgctggcaaacaaggcttttctccaagggatatttatagtctcaaaacacacaattactttacagttagggtgagtttccttttgtgctgttttttaaaataataatttagtatttgtatctcttatagaaatccaagcctatcatgtaaaatgtagctagtattaaaaagaacagattatctgtcttttatcgcacattaagcctctatagttactaggaaatattatatgcaaattaaccggggcaggggagtagccgagcttctcccacaagtctgtgcgagggggccggcgcgggcctagagatggcggcgtcggatcgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgcccctcctttccgtttcagagctaggccagcatgaggatcacccatgcctgcagggcctagcaagttgaaataaggctagtccgttatcaacttggccagcatgaggatcacccatgcctgcagggccaagtggcaccgagtcggtgcttttttt

SEQ ID NO: 56 - DNA, 174 bp, Artificial Sequence
  Sequencing product
acctagtgtgcctagaggggtgtgacacacattttcggacaatttgaataaagggcacggtgcgtgcgcgcggtgactattccagcttctggcttccagcacgcacgactggttccgggattctcgcaccgcgcaccgcacggagccggctgctgcgggctgggagggcgccta

SEQ ID NO: 57 - DNA, 671 bp, Artificial Sequence
  Sequencing product
gttttgagactataaatatcccttggagaaaagccttgtttgttgaataaagggcagtttcagagctaggccagcatgaggatcacccatgcctgcagggcctagcaagttgaaataaggctagtccgttatcaacttggccagcatgaggatcacccatgcctgcagggccaagtggcaccgagtcggtgctttttttgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcaaagcggcaggaggtttcagagctaggccagcatgaggatcacccatgcctgcagggcctagcaagttgaaataaggctagtccgttatcaacttggccagcatgaggatcacccatgcctgcagggccaagtggcaccgagtcggtgctttttttgttttagagctagcgaattcggctccggtgcccgtcagtgggcagagcgcacatcgcccacagtc

SEQ ID NO: 58 - DNA, 219 bp, Artificial Sequence
  Sequencing product
gagactataaatatcccttggagaaaagccttgtttgttgaataaagggcagtttcagagctaggccagcatgaggatcacccatgcctgcagggcctagcaagttgaaataaggctagtccgttatcaacttgggccaacatgaggatcacccatgtctgcagggcccaagtggcaccgagtcggtgctttttttgttttagagctagcgaattcggc

SEQ ID NO: 59 - DNA, 469 bp, Artificial Sequence
  Sequencing product
atcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccactctttcccaagagttggtagagtgtttcagagctaggccagcatgaggatcacccatgcctgcagggcctagcaagttgaaataaggctagtccgttatcaacttggccagcatgaggatcacccatgcctgcagggccaagtggcaccgagtcggtgctttttttctagcgcggccgcagtatgatacacttgatgaagccgaattctgcagatatccatcacactggcggccgctcgagcatgcatctagagggcccaattcgcc

SEQ ID NO: 60 - DNA, 423 bp, Artificial Sequence
  Sequencing product
gtggaaaggacgaaacaccgttgaataaagggcagtttcagagctaggccagcatgaggatcacccatgcctgcagggcctagcaagttgaaataaggctagtccgttatcaacttggccagcatgaggatcacccatgcctgcagggccaagtggcaccgagtcggtgcaacaaagcaccagtggtctagtggtagaatagtaccctgccacggtacagacccgggttcgattcccggctggtgcacaaagcggcaggaggtttcagagctagggccaacatgaggatcacccatgtctgcagggccctagcaagttgaaataaggctagtccgttatcaacttgggccaacatgaggatcacccatgtctgcagggcccaagtggcaccgagtcggtgctttttttaagcttggcttgaat

SEQ ID NO: 61 - DNA, 244 bp, Artificial Sequence
  Sequencing product
atgcttaccgtaacttgaaagtatttcgatttcttggctttatatatcttgtggaaaggacgaaacaccgttgaataaagggcagtttcagagctaggccagcatgaggatcacccatgcctgcagggcctagcaagttgaaataaggctagtccgttatcaacttgggccaacatgaggatcacccatgtctgcagggcccaagtggcaccgagtcggtgctttttttaagcttggcttgaat